(19) United States
(12) Patent Application Publication
(10) Pub. No.: US 2025/0312329 A1
(43) Pub. Date: Oct. 9, 2025

(54) MODIFIED RELEASE NICORANDIL COMPOUND FORMULATIONS

(71) Applicant: AUXILIUS PHARMA SPOLKA Z OGRANICZONA ODPOWIEDZIALNOSCIA, Warsaw (PL)

(72) Inventors: Denny Johan Marijn Van Den Heuvel, Beuningen (NL); Johannes Jan Platteeuw, Boxtel (NL); Uwe Tigor, Saratoga Springs, NY (US); Dawid Sebastian Chabowski, Warsaw (PL)

(21) Appl. No.: 18/966,830

(22) Filed: Dec. 3, 2024

Related U.S. Application Data
(63) Continuation of application No. 18/408,490, filed on Jan. 9, 2024, now abandoned.
Application No. 18/408,490 is a continuation-in-part of application No. PCT/IB2022/056398, filed on Jul. 11, 2022.

(30) Foreign Application Priority Data
Jul. 10, 2021 (PL) P.438438

(51) Int. Cl. A61K 31/455; A61J 1/03; A61J 1/14; A61K 9/20; A61K 9/28; A61K 9/48

(52) U.S. Cl. CPC A61K 31/455; A61J 1/035; A61J 1/1468; A61K 9/2013; A61K 9/2018; A61K 9/2054; A61K 9/2059; A61K 9/2846; A61K 9/4866

(57) ABSTRACT

Provided are compositions comprising nicorandil compound(s) in a controlled release formulation that releases at least about 30% of the nicorandil compound(s) into the intestine of human subjects. Such compositions can include a gastric acid dissolution susceptible component (GADSC) and a gastric acid dissolution resistant component (GADRC), wherein upon maintaining the composition in contact with a pH 1.2 dissolution media for a period of about 2 hours (1) the GADRC releases a statistically significantly smaller proportion of the one or more nicorandil compounds in the GADRC than the proportion of one or more nicorandil compounds release from the GADSC and (2) the composition releases no more than 50% of the one or more nicorandil compounds contained in the composition. The invention further provides methods of product such compositions and therapeutic uses of compositions, e.g., comprising once-daily administration of the composition to treat one or more diseases or conditions, including angina.

### PRIORITY/RELATED APPLICATION

This patent application is a continuation of U.S. application Ser. No. 18/408,490, filed Jan. 9, 2024, titled “Modified Release Nicorandil Compound Formulations,” which is a continuation-in-part of PCT application number PCT/IB2022/056398, filed Jul. 11, 2022, titled “Modified Release Nicorandil Compound Formulations,” which claims the benefit of priority to Polish Patent Application P.438438, filed Jul. 10, 2021, titled “Nicorandil Compound Formulations for Effective Once Daily Treatment Regimens.” This application claims the benefit of priority to, and incorporates by reference the entirety of, the above-referenced priority applications.

### FIELD OF THE INVENTION

This application relates to pharmaceutical compositions comprising nicorandil or related compounds in modified-release formulations and related methods of production and use.

### BACKGROUND OF THE INVENTION

Nicorandil (N-[2-(Nitro-oxy) ethyl]-3-pyridine carboxamide), a nicotinamide ester, is an active pharmaceutical ingredient having both a nicotinamide group and a nitrate group and is characterized by a unique dual mechanism of action including both nitrate-like and potassium-channel-agonist properties. The nitrate-like action of nicorandil causes both venous and epicardial coronary artery dilation, and the potassium channel opening dilates both peripheral arterioles and coronary microvessels. Due to its potent coronary vasodilation ability, nicorandil is especially suitable for use in the prevention and treatment of chronic stable angina pectoris (CSAP). This type of angina pectoris is the most frequent (and often the first) manifestation of coronary artery disease.

Chronic-stable angina pectoris is traditionally seen as caused by underlying atherosclerotic coronary artery disease of the macrovascular coronary flow bed. However, angina symptoms are also frequently caused by coronary microvascular disease (CMD) and dysfunction and patients with microvascular angina constitute a sizeable proportion of all cases of stable angina undergoing diagnostic coronary angiography and of those with persisting angina after successful coronary revascularization. Although initially described as angina on the basis of CMD and in the absence of obstructive coronary artery disease it is now acknowledged that macrovascular and microvascular angina frequently appear to coexist. Kaski J C et al. Circulation. 2018 Oct. 2; 138(14):1463-1480.

Cardinal manifestations of CMD include angina, exertional dyspnea, and possibly heart failure. Angina occurs in approximately 30% to 60% of patients with CMD and is indistinguishable from angina caused by obstructive coronary artery disease (CAD): It is commonly precipitated by exertion and relieved by rest. Patients may also exhibit a gradual decrease in exercise tolerance or dyspnea on exertion. Taqueti V R et al. J Am Coll Cardiol. 2018 Nov. 27; 72(21):2625-2641.

Nicorandil was first discovered in Japan by Chugai Pharmaceutical for symptomatic treatment of patients with stable angina pectoris who are inadequately controlled or have a contraindication or intolerance to the first-line anti-angina agents, such as beta-blockers, CCBs, and nitrates. Approval was later granted in Australia, the United Kingdom, and the European Union (EU) for the same indication and drugs containing nicorandil have also been approved for use in the Philippines, India, South Korea, and Taiwan. In 2020, it was recommended by the European Society of Cardiology guidelines as a second-line drug in patients with chronic coronary disease. Nicorandil is not approved for use in the United States. In Europe, a leading nicorandil product is IKOREL® marketed by Sanofi (e.g., under Irish License Number PA0540/102/002), a leading international pharmaceutical company, which, as recognized by the European Medicine Agency, is separately marked by Merck KGaA under the brand name DANCOR®. A listing of other nicorandil pharmaceutical products and associated countries where they are approved can be found at drugs.com (under a search of nicorandil) (site visited Jul. 7, 2021).

Nicorandil has a dual mechanism of action, which ultimately leads to relaxation of vascular smooth muscle in the vascular wall. Nicorandil's potassium channel opening action provides arterial vasodilation, reducing afterload. The nitrate component promotes venous relaxation and a reduction in preload. Furthermore, the activation of the potassium channel appears to lead to cardioprotective effects mimicking ischemic pre-conditioning. Nicorandil increases coronary blood flow and improves angina symptoms both in intensity and frequency on par with first-line agents.

Nicorandil has a very short elimination half-life of approximately 1 hour. Due to its short elimination half-life, and the limitations of current nicorandil products, patients typically administer nicorandil multiple times a day to maintain effectiveness. The half-life and moisture and temperature sensitivity also makes it extremely difficult to select proper excipients, polymers, and blending technology that will produce an effective and stable once-a-day (“QD” or “QiD”) formulation. Currently marketed oral formulations are immediate-release formulations that are prescribed to take twice daily (“BiD”) usually in starting amounts of 5 mg or 10 mg. IKOREL®, for example, has been determined to be effective based on such a treatment regimen. Studies of IKOREL pharmacokinetics and efficacy indicate that after each dose about 50% of nicorandil in the initially administered composition is detectable in plasma three hours after administration, essentially 0% remains five hours after administration, and efficacy is maintained for at least 6 hours post administration. Frydman A M et al. Am J Cardiol. 1989 Jun. 20; 63(21):25J-33J and Frampton J et al. Drugs. 1992 October; 44(4):625-55.

Nicorandil has been well studied in the clinic. For example, the Impact of Nicorandil in Angina (“IONA”) study was a randomized placebo-controlled trial of 5,126 patients with stable angina followed up for an average of 1.6 years, which showed a reduction in the composite endpoint of death caused by coronary heart disease, non-fatal MI or unplanned hospital admission with chest pain in patients treated with nicorandil compared to placebo. However, the IONA studied failed to result in demonstration of any difference in the secondary outcome of coronary heart disease death or non-fatal myocardial infarction (MI), and the individual components of the composite endpoint did not differ significantly between the two treatment groups and nicorandil also had no effect on the distribution of functional Canadian Cardiovascular Society grading of angina at the end of the study. Nonetheless, more recent multi-center observational data from a total of 2,558 patients treated with nicorandil and controls subjected to propensity score matching from the Japanese Coronary Artery Disease (JCAD) study provided additional evidence that nicorandil might confer a degree of long-term cardioprotection for patients with stable angina; demonstrating a 35% reduction in all-cause mortality and 56% reduction in cardiac death in patients treated with nicorandil over an average 2.7 years. Among the proposed cardioprotective mechanisms for nicorandil include K+ATP channel activation of myocardial mitochondrial ischemic preconditioning, protection against long-term endothelial dysfunction, stabilization of atherosclerotic plaques, and other ancillary properties, including antiplatelet effects. Preliminary evidence also suggests that nicorandil is effective in addressing angina symptoms in microvascular angina (MVA) patients.

Given such positive effects, it is not surprising that in the five decades since nicorandil's initial approval, several researchers have developed and described several proposed formulations to serve as QD nicorandil formulations. However, to this day there is no regulatory authority-approved formulation for a once-a-day administration of nicorandil despite the efforts of several researchers to develop such a product.

Reddy et al., for example, published a study regarding making a once-daily sustained release nicorandil tablet in, “Once-Daily Sustained-Release Matrix Tablets of Nicorandil: Formulation and in vitro Evaluation” 4 AAPS PharmSciTech No. 4, 480 (December 2003). The nicorandil formulations studied by Reddy (containing 80 mg nicorandil) were developed with the desire to mimic a theoretical linear (zero-order kinetics) release pattern. According to the authors, the most successful formulation showed high linearity in drug release despite a change in pH level. However, the work performed by Reddy did not ultimately lead to an effective QD nicorandil product, as evidenced by additional research aimed at still reaching the same goal. For example, five years after Reddy, Abdelbary et al. published a study on a different proposed extended release nicorandil tablet product (69 Eur. J. Pharmaceutics and Biopharmaceutics 1019-1028 (2008)). Abdelbary concluded that its preferred formulation extended duration of therapeutic concentration of nicorandil from 4 hours to 8 hours. Similar to the Reddy formulations, the formulations described in Abdelbary also released nicorandil in a linear pattern despite a change in pH in the test conditions used. Like Reddy, the work of Abdelbary also did not lead to any approved QD nicorandil product researchers continued thereafter to work on achieving that goal. For example, Pahade et al. also published a study, “Formulation and Development of Sustained Release Matrix Tablet of Nicorandil,” 4 Int'l J. Pharm. Sci. Rev. and Research No. 1 107-111 (September-October 2010). The Pahade formulations are also released in a linear pattern with no demonstration of a change in release due to pH.

At about this same time, Korean patent document KR 2010 0060595 (to Hanall Biopharma) published, describing a pulsatile release formulation for treating angina pectoris, allegedly suitable for once or twice per day administration, the formulation comprising an inner immediate release compartment (e.g., a core) comprising nicorandil and a stabilizing agent, a release suppression compartment/release suppression layer surrounding the inner immediate release compartment without nicorandil, and an outer immediate release compartment/layer of nicorandil (and, further, a method for manufacturing the same). According to certain embodiments the formulation can repeat the layered immediate release and release suppression components to yield a repeating pulsatile release formulation. Data show 100% release of nicorandil from a two-pulse formulation (FIG. 1) by about 6 hours. Data show 100% release of nicorandil from a three-pulse formulation (FIG. 2) by about 11 hours.

A year after the publication of Pahade, Tamilvanan et al. published a study involving yet another proposed once daily nicorandil tablet, which the authors describe as departing from and attempting to improve over the work of Reddy et al., supra, by using formulation techniques that were known to lead to sustained release tablets (37 Drug Dev. Industrial Pharmacy No. 4: 436-445 (2011)). All of the ten sustained release tablet formulations described in Tamilvanan exhibited a linear release profile despite a change in pH used in test conditions, and the preferred formulation reportedly maintained a minimum effective concentration of nicorandil for allegedly at least 8 hours. Tamilvanan concluded that the duration and intensity of nicorandil released, in vitro, from the optimized and already commercial NIKORAN® OD SR (“once daily sustained release”) tablets (Torrent Labs) was “comparable” to the best performing of the ten formulations the researchers developed (despite higher Cmax and AUC values obtained with the optimized test tablet formulation). Tamilvanan appears to reach other contradictory and confusing conclusions regarding these studies. For example, the authors concluded that “the dosage form does not show an in vivo sustained release profile and the in-vitro-in-vivo correlation is poor.” The authors also suggest that the plasma levels “remained above the minimum effective concentration” for at least 8 hours after ingestion, citing earlier studies, but present data reflecting plasma concentration for the optimized test product and NIKORAN® OD of less than 10 ng/mL.

Even despite the reportedly favorable performance of NIKORAN® OD compared to the best of ten sustained release formulations developed and tested by Tamilvanan et al. (and superior performance to most if not all of the other sustained released formulations) NIKORAN® OD has, to date, only been approved for pharmaceutical use in India. The limited development of NIKORAN® OD may also be due to the fact that the product is only associated with a five-day storage time under refrigerated conditions (at 2-8° C.), reflecting that achieving both a meaningfully modified release formulation of nicorandil while maintaining nicorandil stability may be particularly difficult for even highly qualified and experienced drug manufacturers and researchers.

Perhaps in view of this fact, and, in any event, apparently still not satisfied with any of the preceding work, Kim et al. later published yet another study on an extended release nicorandil tablet (10 Asian J. Pharm. Sciences 1108-113 (2015)), similarly reporting a linear pattern of nicorandil release from tested formulations (apparently nearly unaffected by pH). This work, also, however, did not lead to any new marketed nicorandil product suggesting that the results were either not deemed to be a sufficient improvement over the prior art to support further development or failed on other ground in subsequent testing, such as in stability characteristics.

More recently, patent document (publication) WO 2021/094902 to Dr. Reddy's Laboratories, Ltd (May 20, 2021) discloses a capsule formulation for allegedly once-daily administration comprising immediate and extended release mini-tablets of nicorandil (e.g., thus providing a dual-release composition), wherein the extended release mini-tablets of nicorandil comprise a rate controlling polymer, wherein the total amount of nicorandil present in the composition is 40 mg. Disclosure focuses on the accompaniment of nicorandil with metaprolol. In composition(s) disclosed there, about 90% of nicorandil is released within about 6-8 hours after administration (as determined by dissolution studies).

Despite these various attempts to create an effective QD nicorandil product, none of these efforts have led to a successfully approved (safe and effective) once-a-day (QD) nicorandil product to date or, in fact, any product that significantly improves over the performance of NIKORAN® in terms of controlled release of nicorandil and that exhibits favorable stability characteristics. This collective failure to develop QD nicorandil formulations despite numerous attempts at doing so demonstrates that developing nicorandil formulations that effectively improve over current on-market nicorandil products, e.g., in achieving reliable and stable QD nicorandil products requires the exercise of inventive ingenuity.

### SUMMARY OF THE INVENTION

This document includes a section entitled “CONSTRUCTION PRINCIPLES AND DESCRIPTION OF SELECT TERMS” that readers are encouraged to consult to help properly interpret the disclosure provided in this section and elsewhere here. That section includes a list of acronyms frequently used in this disclosure.

This “Summary of the Invention” section (“Summary”) briefly describes the elements and characteristics of selected illustrative embodiment(s) of the invention. The brief summaries of such embodiments provided here are primarily intended to illustrate the nature of the invention and, accordingly, the content of this Summary is not intended to be all-inclusive, and the scope of the invention is not limited to, or by, the exemplary aspects of the invention provided in this section. Any of the aspects of the invention described in this section can be combined with any other aspect described in this or any other aspect of this disclosure.

The invention provides new pharmaceutical compositions comprising an effective amount of one or more nicorandil compounds in a controlled release formulation, wherein the controlled release formulation provides for detectable or significant release of nicorandil from the formulation for 12, 14, 16, or 18 hours post administration to a human patient. The compositions typically include a first release component and a second release component, where the first release component is significantly more susceptible to releasing nicorandil compounds at high pH (gastric acid-like) conditions than the second release component and the second release component releases relatively little (e.g., less than 20%, less than 15%, or less than 10%) of the nicorandil compounds in the second release component at/after 2, 4, or 6 hours of contact with a gastric acid like dissolution media (e.g., a media with a pH of 1.2). In aspects, the first release component comprises multiple dosage forms (e.g., tablets), the second release component comprises multiple dosage forms (e.g., pH-responsive polymer coated tablets), and both types of dosage forms are contained in a vehicle/delivery system (e.g., a capsule). Aspects include such compositions being stored under conditions that detectably or significant promote stability and, in aspects, achieve certain stability targets, purity targets, or both (with low moisture excipients, with desiccants, with environment-isolated packaging (e.g., blister packages) or a combination thereof). Aspects of the invention further include methods of using such compositions, e.g., to modulate various physiological conditions (such as in vivo nitric oxide concentration, blood vessel diameter, etc.) or to treat various conditions, such as one or more forms of angina.

In one exemplary embodiment of such a composition, the invention provides a pharmaceutically acceptable composition comprising a therapeutically effective amount of one or more nicorandil compounds collectively contained in a controlled release formulation comprising a gastric acid dissolution susceptible component (GADSC) and a gastric acid dissolution resistant component (GADRC), wherein upon maintaining the composition in contact with a pH 1.2 dissolution media for a period of about 4 hours or about 6 hours (1) the GADRC releases a statistically significantly smaller proportion of the one or more nicorandil compounds in the GADRC than the proportion of one or more nicorandil compounds release from the GADSC and (2) the composition releases no more than 50% of the one or more nicorandil compounds contained in the composition.

In a further aspect, the GADRC also or alternatively releases no more than 20% of the one or more nicorandil compounds contained in the GADRC upon about 4 hours or about 6 hours of contact with a dissolution media having a pH of 1.2.

In a further aspect, the GADSC also or alternatively comprises a controlled release formulation comprising an amount of one or more release retardant/resistant polymers that permits the release of at least 33% and the retention of at least about 5% of the one or more nicorandil compounds initially present in GADSC 6 hours after contact with a dissolution media having a pH of about 1.2 or a dissolution media having a pH of about 6.8.

In an embodiment, the GADRC also or alternatively comprises an amount of one or more pH responsive polymers that is effective to release at least 33% of the one or more nicorandil compounds initially present in the GADRC 6 hours after contact with a dissolution media having a pH of about 6.8 and no more than 20% of the one or more nicorandil compounds initially present in the GADRC at 6 hours contact with a dissolution media having a pH of 1.2.

In aspects, the GADSC also or alternatively comprises a first portion of the therapeutically effective amount of the one or more nicorandil compounds and the GADRC comprises a second portion of the therapeutically effective amount of the one or more nicorandil compounds, wherein the first portion comprises 30-70% of the one or more nicorandil compounds in the composition.

In aspects, the one or more nicorandil compounds is nicorandil. In aspects, the effective amount of nicorandil in the composition is 15-90 mg of nicorandil, 15-75 mg of nicorandil, or 15-60 mg of nicorandil (e.g., 25-100, 30-90, 30-120, 35-105, or 35-70 mg).

In embodiments, the composition also or alternatively comprises 3 separate dosage forms made up of 1-5 or 2-5 GADSC dosage forms and 1-5 or 2-5 GADRC dosage forms, wherein the separate dosage forms are contained in a suitable capsule.

In embodiments, the GADSC, GADRC, or both the GADSC and GADRC, also or alternatively comprise(s) a matrix formulation comprising (1) about 25-45% of a release retardant polymer component and (2) about 40-70 a binder component.

According to aspects, most of the excipients in the composition also or alternatively have a moisture content of less than 6%.

In aspects, the amount of the one or more nicorandil compounds released from the GADSC when the GADSC when maintained in contact with a dissolution media of pH 6.8 is also or alternatively in accordance with the following release profile: (1) about 0.5-25% of the one or more nicorandil compounds being released from the GADSC at 0.25 hours; (2) about 11.5% to about 44.5% of the one or more nicorandil compounds being released at 1 hour; (3) about 25% to about 60% of the one or more nicorandil compounds being released at 2 hours; (4) about 60-92.5% of the one or more nicorandil compounds being released at 6 hours; (5) about 71% to about 98% of the one or more nicorandil compounds being released at 8 hours, and (6) about 83% to about 100% or the one or more nicorandil compounds being released at 14 hours.

In embodiments, the amount of the one or more nicorandil compounds released from the GADRC when the GADRC is maintained in contact with a dissolution media of pH 6.8 is also or alternatively in accordance with the following release profile: (1) about 0% to about 8.5% of the one or more nicorandil compounds being released at 0.25 hours; (2) about 15.5% to about 29% of the one or more nicorandil compounds being released at 1 hour; (3) about 27% to about 50% of the one or more nicorandil compounds being released at 2 hours; (4) about 65-80%, of the one or more nicorandil compounds being released at 6 hours; (5) about 75% to about 87.5% of the one or more nicorandil compounds being released at 8 hours, and (6) about 83.5% to about 98% of the nicorandil compounds being released at 14 hours.

In embodiments, the GADRC, GADSC, or both, also or alternatively comprise (1) an effective amount of a release retardant polymer component, wherein the release resistant polymer component is at least mostly composed of hydroxypropyl methylcellulose and the release resistant polymer component and the one or more nicorandil compounds are present in a ratio of about 5:1 to about 2:1 and (2) an effective amount of a binder component, wherein the binder component at least most comprises silicified microcrystalline cellulose, and the binder component and the nicorandil compound(s) are present in a ratio of about 4:1 to about 7:1.

According to certain specific embodiments, the invention provides pharmaceutically acceptable composition(s) comprising a therapeutically effective amount of nicorandil compound(s) contained in a controlled release formulation. In aspects, the controlled release formulation(s) provided herein comprise a controlled release gastric acid dissolution susceptible component. In aspects, a controlled release gastric acid dissolution susceptible component of composition(s) provided herein comprise(s) a release retardant polymer. In aspects, controlled release formulation(s) provided by the invention comprise(s) a controlled release gastric acid dissolution resistant component. In aspects, a controlled release gastric acid dissolution resistant component of composition(s) provided herein comprise(s) a release retardant polymer component and a pH responsive release resistant polymer component. In aspects, composition(s) provided by the invention comprise a therapeutically effective amount of nicorandil compound(s) and, upon maintaining the composition(s) in contact with a pH 1.2 dissolution media for a period of about 4 hours, the composition(s) release(s) no more than about 50% of the nicorandil compound(s) contained in the composition(s). In aspects, composition(s) provided herein comprise a therapeutically effective amount of nicorandil compound(s) and a gastric acid dissolution susceptible controlled release component comprising a first part of the therapeutically effective amount of the nicorandil compound(s). In aspects, composition(s) provided herein comprise a therapeutically effective amount of nicorandil compounds and a gastric acid dissolution susceptible controlled release component which releases between about 55% and about 70% of the nicorandil compound(s) initially present in the gastric acid dissolution susceptible component after about 2 hours contact with a dissolution media having a pH of about 1.2. In aspects, composition(s) provided herein comprise a therapeutically effective amount of nicorandil compound(s) and a gastric acid dissolution susceptible controlled release component which releases about 80% to about 100% of the nicorandil compound(s) initially present in the gastric acid dissolution susceptible component about 6 hours after contact with the dissolution media having a pH of about 1.2. In aspects, composition(s) provided herein comprise a therapeutically effective amount of nicorandil compound(s) and a gastric acid solution resistant component comprising a second part of the therapeutically effective amount of the nicorandil compound(s). In aspects, composition(s) provided herein comprise a therapeutically effective amount of nicorandil compound(s) and a gastric acid dissolution resistant component which releases no more than about 20% of the nicorandil compound(s) contained in the gastric acid dissolution resistant component after about 6 hours of contact with a dissolution media having a pH of 1.2. In aspects, composition(s) provided herein comprise a therapeutically effective amount of nicorandil compound(s) characterizable as present in the composition(s) in a first part of the composition(s) and a second part of the composition(s), wherein the amount of nicorandil compound(s) in the first part of the therapeutically effective amount and the amount of nicorandil compound(s) in the second part of the therapeutically effective amount differ by less than ˜10%.

A further embodiment of the invention is a suitable package comprising a composition having any one or more of the above-described characteristics and an aluminum blister pack containing an effective amount of a pharmaceutically acceptable desiccant.

Still a further embodiment of the invention is a method of treating one or more types of angina in a human patient comprising administering an effective amount of a composition of any one or more of the above-described compositions or a composition contained in the package described immediately above, which is administered to patients no more than once per day during at least a part, e.g., most, e.g., all, of a treatment period, which can be, e.g., at least 3, 6, 12, 18, or 24 months in duration.

In embodiments any one or more of these features are also or alternatively combined with any suitable related or alternative aspects provided in the Exemplary Aspects section of this disclosure.

In embodiments any one or more of these features are also or alternatively combined with any suitable related or alternative aspects provided in the Detailed Description section of this disclosure, the Examples, the Figures, or any combination thereof.

### BRIEF DESCRIPTION OF THE DRAWINGS/FIGURES

The drawings/figures provided here, and the associated following brief description of figures, are intended to exemplify certain aspects and principles of the invention without limiting its scope.

FIG. 1 illustrates a nicorandil dissolution release profile obtained from contacting exemplary uncoated (first/general release formulation) nicorandil tablets according to aspects of the invention over a ˜16-hour period with a dissolution media at pH 6.8.

FIG. 2 illustrates a dissolution release profile of exemplary coated (second release/pH responsive release) nicorandil tablets over a ˜18-hour period at pH 6.8.

FIG. 3 illustrates a nicorandil release profile obtained from exemplary uncoated (first/general release) tablets over a ˜14-hour period at pH 1.2.

FIG. 4 illustrates a nicorandil release profile obtained from test exemplary coated (pH responsive release) nicorandil tablets over an ˜18-hour period at pH 1.2.

FIG. 5 illustrates a dissolution release profile for uncoated (first/general release) nicorandil tablets over a 14-hour period at pH 1.2 compared to a target tablet release profile.

FIG. 6 is a graph comparing the dissolution profiles for exemplary coated and uncoated nicorandil tablets of the invention at pH 1.2 and pH 6.8, respectively.

FIG. 7 illustrates a release profile of an exemplary nicorandil capsule containing both exemplary coated and uncoated tablets over a ˜18-hour period at a pH of 6.8.

FIG. 8 is a graph of dissolution profiles of exemplary nicorandil capsules comprising a HPMC capsule and a gelatin capsule at pH 6.8 over ˜16 hours.

FIG. 9 is a graph of dissolution profiles of exemplary nicorandil tablets having different hardness properties under different dissolution conditions.

FIG. 10 is a graph of dissolution profiles of exemplary nicorandil tablets comprising different grades of HPMC in the tablet formulation (HPMC-CR and HPMC-DC2).

FIG. 11 is a graph of the dissolution profiles of exemplary nicorandil capsule formulations maintained in media at pH 6.8 and pH 1.2, respectively, for ˜16 hours.

FIG. 12 is a combined graph of dissolution data for nicorandil capsules (containing both coated and uncoated tablets) maintained at a pH of 6.8 for a period of about 14 hours and coated tablets maintained at a pH of 1.2 for about 2 hours.

FIG. 13 is a graphical representation of actual dissolution data of components of an exemplary nicorandil capsule of the invention over a ˜22-hour period as compared to the expected in-vivo profile for an immediate release (IR) IKOREL© 20 mg nicorandil BiD (40 mg daily) formulation (based on actual dissolution data obtained at pH 1.2).

FIG. 14 is a graphical representation of release of nicorandil coated and uncoated tablets reflecting anticipated in-vivo dissolution of compositions of the invention.

FIG. 15 is a graph of dissolution data obtained from maintaining NIKORAN® OD tablets under pH 6.8 and pH 1.2 conditions for a period of about 24 hours.

FIG. 16 is a graphical comparison of dissolution data of NIKORAN® OD tablets vs. IKOREL® immediate release tablets at pH 6.8.

FIG. 17 is a graph of dissolution assay data for NIKORAN® OD tablets at pH 1.2 and pH 6.8 conditions as compared to coated and uncoated exemplary nicorandil tablets of the invention at pH 6.8 and pH 1.2, respectively, over a period of ˜24 hours.

FIG. 18 is a graph of dissolution data obtained for exemplary coated tablets maintained at pH 1.2 over a period of ˜2 hours, exemplary nicorandil capsules maintained at pH 6.8 over a period of about 16 hours, and NIKORAN® OD tablets maintained a pH of 1.2 over about 10 hours.

FIG. 19 is a graph of nicorandil dissolution release data for uncoated nicorandil tablets at pH 1.2 and pH 6.8, coated nicorandil tablets at pH 1.2, and NIKORAN® OD tablets maintained at pH 1.2 over a period of about 18 hours, reflecting the expected pattern/profile of in-vivo dissolution of such compositions.

FIG. 20 is a graph of the percentage of initial formulation nicorandil available from NIKORAN® OD tablets and uncoated nicorandil tablets maintained at pH 1.2.

FIG. 21 is a graph of the percentage of initial formulation nicorandil available from NIKORAN® OD tablets, uncoated nicorandil tablets, and coated nicorandil tablets of the invention maintained at pH 1.2 and from coated nicorandil tablets maintained at pH 6.8, reflecting expected in-vivo dissolution profiles of such compositions.

FIG. 22 is a schematic representation of an exemplary production method for producing coated (and uncoated) nicorandil tablets according to aspects of the invention.

FIG. 23 is a depiction of nicorandil (NCD) and three nicorandil-associated impurities (impB, impC, and impD) formed by nicorandil hydrolysis in aqueous solution.

FIG. 24 is a spectrograph of HPLC data of various compositions, including exemplary nicorandil capsules of an aspect of the invention maintained under indicated dissolution conditions.

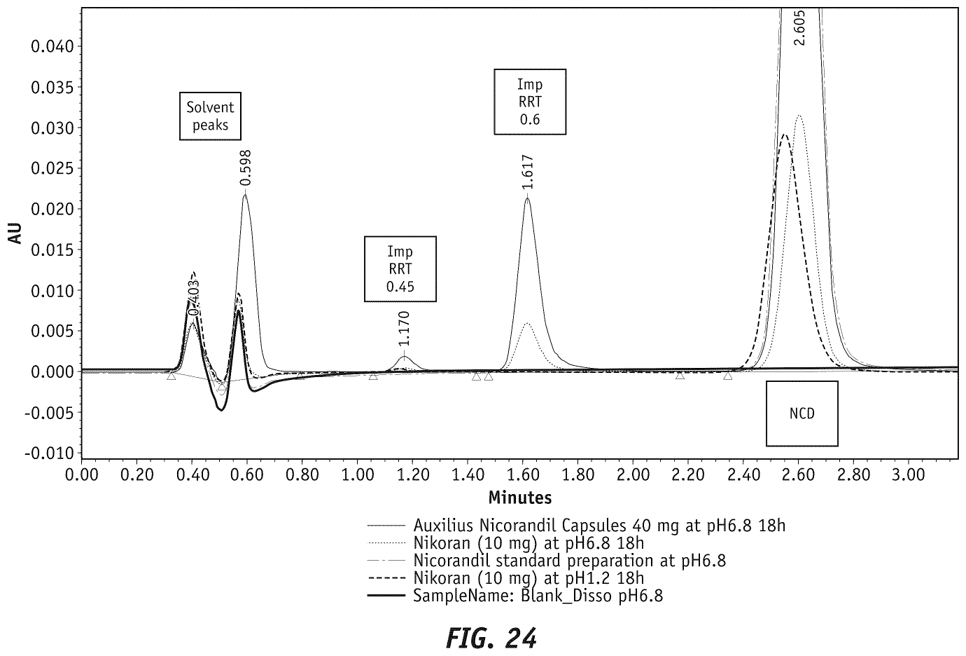

FIG. 25 is a graph of dissolution data obtained for exemplary nicorandil capsules after maintenance under different indicated stability testing conditions.

FIG. 26 is an illustration of the sections/anatomy of the human gastrointestinal tract along with typical pH conditions and drug residence times associated therewith.

### EXEMPLARY ASPECTS OF THE INVENTION

The following is a non-limiting list of exemplary aspects of the invention, which illustrates embodiments of the invention in a summary form to aid readers in quickly understanding the overall scope of the invention.

It is intended that these listed exemplary aspects begin with the first listed aspect (ASPECT 1) and thereafter be numbered sequentially and incrementally by the inclusion of a reference placed near or at the end of the listed aspect (ASPECT 2, ASPECT, 3, etc.). Similar to patent claims, these aspects of the invention listed in the paragraphs of this section may make reference to (depend on/from) one or more other aspects referenced in other paragraphs. Readers will understand that such references mean that the features/characteristics or steps of such referenced aspects are incorporated into/combined with the referring aspect. For example, if an aspect in a paragraph (e.g., a paragraph indicated by text at the end of the paragraph as aspect 2) refers to another aspect by one or more aspect numbers (e.g., aspect 1 or “any one of aspects 1-3”), it will be understood to include the elements, steps, or characteristics of such referenced aspects (e.g., aspect 1) in addition to those of the aspect in which the reference is made (e.g., if aspect 2 refers to aspect 1, it provides a description of an object or method including the features of both aspect 1 and aspect 2). Reference to ranges of aspects should be interpreted as referencing all such aspects individually, each as unique embodiments of the invention, and in combination with one another as unique embodiment(s) of the invention, according to the presentation provided of such aspects unless such an aspect within such a referenced range is either contradictory or non-sensical. If contradicted, reference to the contradictory aspect should be excluded. In case of a missing aspect reference or repeated aspect reference, the order of placement of the actual recited aspect in the list that is associated with the repeated aspect reference or missing aspect reference will control (e.g., if there is an unlabeled aspect located between a first aspect labeled ASPECT 1 and a third aspect labeled ASPECT 2, the unlabeled aspect should be treated as ASPECT 2, and the aspect labeled as ASPECT 2 treated as ASPECT 3, etc.), and all numbering in the list (including all references to aspects in the list) be interpreted as accordingly modified (e.g., if the fourth aspect in such list was labeled as ASPECT 3, it should be interpreted as being labeled as ASPECT 4, and if such aspect referred to “any one or both of aspect 1 or aspect 2,” it should be read as referring to “any one or more of aspects 1-3”). Similarly, if an aspect is misnumbered (e.g., by a number in the sequence being skipped or otherwise missing), readers will similarly construe this list of aspects according to the order of placement of the recited aspects, over the numerical references. Further, if one or more of the listed exemplary aspects of the invention in this section fails to reference any other aspects of the invention, such aspect, uncontradicted, should be interpreted as applying to or as capable of being incorporated into, any one or more other exemplary aspect(s) provided in this section.

In a first aspect, the invention provides a pharmaceutically acceptable composition comprising a therapeutically effective amount of one or more nicorandil compounds collectively contained in a controlled release formulation comprising a gastric acid dissolution susceptible component (GADSC) and a gastric acid dissolution resistant component (GADRC), wherein upon maintaining the composition in contact with a pH 1.2 dissolution media for a period of about 2 hours (1) the GADRC releases a statistically significantly smaller proportion of the one or more nicorandil compounds in the GADRC than the proportion of one or more nicorandil compounds released from the GADSC and (2) the composition releases no more than 50% of the one or more nicorandil compounds contained in the composition (aspect 1).

In another case, the invention provides a pharmaceutically acceptable composition comprising an effective amount of one or more nicorandil compounds contained in at least one dosage form of a first controlled release formulation and a second dosage form of a second controlled release formulation, wherein the first controlled release formulation, second controlled release formulation, or both comprise a core formulation comprising about 25-50% of a highly compactible binder component, about 5-25% of a disintegrant binder, and about 20-45% of a controlled release polymer (e.g., a cellulose ether derivative polymer), and at least some of the second controlled release formulation comprises or further comprises a pH responsive coating comprising an effective amount of a pH-resistant release controlling polymer component and, optionally further comprising an effective amount of a pharmaceutically acceptable plasticizer, wherein the pH-release resistant polymer constitutes at least about 65% of the coating (aspect 2).

In another facet, the invention provides a pharmaceutically acceptable composition comprising a controlled release polymer component, a pH responsive polymer component, and a therapeutically effective amount of one or more nicorandil compounds, wherein the controlled release polymer component and the one or more nicorandil compounds are present in the composition in a ratio of about 2.5:1 to about 4.5:1, and wherein at least part of the formulation (e.g., 33-66% of the composition, such as 40-60, 45-55, or about 50% of the composition) is coated with a pH responsive coating that is at least mostly composed of the pH responsive polymer (e.g., a mostly, generally, or entirely methacrylic acid coating) and the pH responsive polymer significantly reduces the release of the one or more nicorandil compounds when the composition is maintained in a pH 1.2 dissolution media for 2, 4, 5, 6, or 8 hours (aspect 3).

Another embodiment is a pharmaceutically acceptable composition comprising an amount of one or more nicorandil compounds that is effective in modulating or treating a nicorandil-treatable condition, at least ˜33% of which is contained in (e.g., ≥40% of which, or ≥˜50% of which contained in) a pH responsive formulation that releases no more than about 20% (e.g., no more than ˜15% or no more than ˜10%) of the nicorandil in the pH responsive dosage form when the pH responsive formulation is maintained in pH 1.2 dissolution media for 2-8 (e.g., 2-6, 2-5, 2-4, or 2-3) hours (aspect 4).

Yet a further embodiment is a pharmaceutically acceptable composition comprising a therapeutically effective amount of one or more nicorandil compounds contained in a controlled release matrix/formulation with a portion of the matrix formulation corresponding to 35-65% of the controlled release matrix/formulation (e.g., 45-55% of the controlled release matrix/formulation) being protected by a pH responsive polymer component (e.g., a mostly, generally, or entirely methacrylic acid coating) that permits no more than 20% of the nicorandil compound(s) to be released from the portion when the portion is maintained in a dissolution media at pH 1.2 for 2-6 hours and wherein (1) the ratio of the pH responsive polymer to the remainder of the composition is about 1:20 to about 1:10 (e.g., 1:18 to 1:12, 1:17 to 1:13, or about 1:15), (2) the ratio of the pH responsive polymer to the one or more nicorandil compounds is about 1:2 to about 3:4, such as about 3:5, or (3) the amount of pH responsive polymer in relationship to the remainder of the composition and nicorandil satisfies the requirements of both ratio (1) and ratio (2) (aspect 5).

An additional aspect of the invention provides a pharmaceutically acceptable composition comprising a therapeutically effective amount of one or more nicorandil compounds and a controlled release formulation that is at least mostly composed of (e.g., generally composed of or substantially composed of) a controlled release polymer component and a microcrystalline cellulose (MCC) component (MCCC), wherein the composition is characterized by one or more of (1) the MCCC and the one or more nicorandil compounds being present in a ratio of about 2.5:1 to about 4.5:1, 3:1 to about 4:1, about 3.25:1 to about 4.25:1, e.g., about 3.5:1 and (2) the MCCC and the controlled release polymer component are present in a ratio of about 0.5:1 to about 1.5:1, e.g., about 0.75:1 to about 1.25:1, such as about 1:1 (aspect 6).

Still another facet of the invention is a pharmaceutically acceptable composition comprising a therapeutically effective amount of one or more nicorandil compounds and a controlled release formulation, wherein the composition releases a detectable amount of the one or more nicorandil compounds for a period of at least about 12 hours after administration to a human patient (e.g., containing at least about 2.5-12.5%, 3-15%, or ˜5-10% of the initial nicorandil at about 12 hours after administration) when administered within 1 month after manufacture or upon administration after more than about 1 month of storage in desiccant-fitted aluminum packaging at either 5° C. or 25° C. (and 60% relative humidity (RH)) (e.g., after 3 months, 6 months, 12 months, 18 months, 24 months, 30 months, or 36 months at one or both conditions), wherein the composition maintains at least about 97% (e.g., ≥98%, ≥98.5%, or ≥99%) of the initial amount of the one or more nicorandil compounds in the composition after such a period of storage (aspect 7).

Further provided is a pharmaceutical composition comprising an amount of one or more nicorandil compounds that is therapeutically effective for once-daily administration that is contained in a first controlled release component and a second controlled release component, wherein the first controlled release component and the second controlled release component are physically separated from one another in the composition and exhibit a statistically significant difference rate of release of the nicorandil compound(s) at one or more pH conditions (e.g., at pH 1.2), and wherein the first controlled release component and the second controlled release component are contained in a delivery facilitating component (a physical carrier—e.g., a capsule) (aspect 8).

Even further provided is a pharmaceutical composition comprising an amount of one or more nicorandil compounds that is therapeutically effective for once-a-day administration that is contained in a plurality of a first controlled release dosage forms and a plurality of second controlled release dosage forms, each of the controlled release dosage forms (regardless of type) being physically separate from each of the other controlled release dosage forms in the composition, all of the controlled release dosage forms being contained in a single delivery facilitating component (e.g., a capsule), wherein (1) a first controlled release dosage form exhibits a statistically significantly different release of the nicorandil compound(s) at least one pH condition (e.g., at pH 1.2), (2) the composition when administered to human patients is at least about as therapeutically effective (or exhibits statistically similar efficacy/results as, e.g., is non-inferior to) or is detectably or significantly more effective than administration of twice daily immediate release nicorandil (e.g., BID administration of Ikorel tablets) (aspect 9).

Another aspect is a pharmaceutically acceptable composition comprising a once-daily therapeutically effective amount of one or more nicorandil compounds, wherein the therapeutically effective amount of the one or more nicorandil compounds is contained in a first pH independent controlled release dosage form and a second pH-responsive/sensitive controlled release dosage form, wherein (1) the pH-responsive controlled release dosage form exhibits a significantly delayed release of nicorandil compound(s) as compared to the pH-independent controlled release dosage form in the stomach of human patients upon administration or (and/or) (2) the pH-responsive controlled release dosage form exhibits a more prolonged release of nicorandil compound(s) under one or more intestinal pH conditions than the pH-independent controlled release dosage form under the one or more intestinal pH conditions (aspect 10).

In another case the invention provides a pharmaceutically acceptable composition comprising a therapeutically effective amount of one or more nicorandil compounds that are therapeutically equivalent (or non-inferior, as determined by prevailing regulatory authority trial standards) or superior to nicorandil with respect to one or more nicorandil-treatable conditions (e.g., one or more forms of angina) and a controlled release formulation wherein the controlled release formulation causes at least about 30% of the initial amount of the one or more nicorandil compounds in the composition to be present at 5 hours post administration of the composition to a human patient (aspect 11).

In a further facet, the invention provides a pharmaceutically acceptable composition comprising one or more nicorandil compounds that is contained in a first controlled release formulation component and a second controlled release formulation component, wherein at about 6 hours after administration of the composition to human patient(s) the composition retains an average of at least about 20% of the initial amount of the one or more nicorandil compounds (i.e., at least about 20% of the initial amount of nicorandil remains undelivered/unreleased) (aspect 12).

In still another embodiment the invention provides a pharmaceutically acceptable composition comprising a therapeutically effective amount of one or more nicorandil compounds wherein the composition contained in a first pH independent controlled release formulation and a second pH responsive controlled release formulation, wherein the composition, when administered to human patients, (a) releases (e.g., has released) about 20% to about 50% of the initial amount of the one or more nicorandil compounds in the composition about 2 hours after administration, (b) releases about 50% to about 80% of the initial amount of the one or more nicorandil compounds in the composition about 6 hours after administration, and (c) and at 14 hours after administration has released no less than 75% of the initial amount of the one or more nicorandil compounds in the composition (aspect 13).

A further embodiment is a pharmaceutically acceptable composition comprising a therapeutically effective amount of one or more nicorandil compounds that are therapeutically equivalent or superior to nicorandil with respect to one or more nicorandil-treatable conditions (e.g., one or more forms of angina) and a controlled release formulation wherein once daily administration of the composition to human patients is bioequivalent to twice daily administration of Ikorel immediate release nicorandil tablets (aspect 14).

Another aspect is a pharmaceutically acceptable composition comprising a therapeutically effective amount of one or more nicorandil compounds that are therapeutically equivalent or superior to nicorandil with respect to one or more nicorandil-treatable conditions (e.g., one or more forms of angina) and a controlled release formulation wherein once daily administration of the composition to human patients results in a statistically significantly similar or statistically significantly superior improvement in one or more nicorandil-associated conditions (e.g., delayed angina symptoms during exercise, dilation of blood vessels, or both) as compared to twice-daily administration of Ikorel IR nicorandil tablets (aspect 15).

A further aspect is a pharmaceutically acceptable composition comprising a therapeutically effective amount of one or more nicorandil compounds that are therapeutically equivalent or superior to nicorandil with respect to one or more nicorandil-treatable conditions (e.g., one or more forms of angina) and a controlled release formulation wherein once daily administration of the composition to human patients is effective to treat one or more forms, conditions, or symptoms of angina in a statistically significant number of patients (aspect 16).

Also provided is a pharmaceutically acceptable composition comprising a therapeutically effective amount of one or more nicorandil compounds that are therapeutically equivalent or superior to nicorandil with respect to one or more nicorandil-treatable conditions (e.g., one or more forms of angina) and a controlled release formulation wherein once daily administration of the composition results in an increase exercise duration at 6, 8, 12, 14, or 18 hours after administration that is at least about the same/statistically similar as or significantly superior to twice-daily administration of a similar amount of immediate release nicorandil tablets (e.g., Ikorel tablets BID) at 6, 8, 12, 14, or 18 hours after administration (aspect 17).

A further embodiment is a pharmaceutically acceptable composition comprising a therapeutically effective amount of one or more nicorandil compounds that are therapeutically equivalent or superior to nicorandil with respect to one or more nicorandil-treatable conditions (e.g., one or more forms of angina) and a controlled release formulation wherein once daily administration of the composition results in an increase in average exercise duration by about 13% to 35% from baseline at 6, 8, 12, 14, or 18 hours after administration (aspect 18).

Provided further is a pharmaceutically acceptable composition comprising a therapeutically effective amount of one or more nicorandil compounds that are therapeutically equivalent or superior to nicorandil with respect to one or more nicorandil-treatable conditions (e.g., one or more forms of angina) and a controlled release formulation wherein once daily administration of the composition results in a statistically similar/approximately the same or statistically superior improvement in time to onset of angina (e.g., upon exertion) as compared to administration of a similar amount of nicorandil compound(s) by twice-daily administration of an immediate release formulation (e.g., BID administration of Ikorel tablets) at 6, 8, 12, 14, or 18 hours after administration (aspect 19).

Another facet is a pharmaceutically acceptable composition comprising a therapeutically effective amount of one or more nicorandil compounds that are therapeutically equivalent or superior to nicorandil with respect to one or more nicorandil-treatable conditions (e.g., one or more forms of angina) and a controlled release formulation wherein once daily administration of the composition results in delaying the time to onset of angina (e.g., after exertion) by about 20% to 40% from baseline at 6, 8, 12, 14, or 18 hours after administration (aspect 20).

Another exemplary embodiment is a pharmaceutically acceptable composition comprising a therapeutically effective amount of one or more nicorandil compounds that are therapeutically equivalent or superior to nicorandil with respect to one or more nicorandil-treatable conditions (e.g., one or more forms of angina) and a controlled release formulation wherein once daily administration of the composition results in a statistically similar/approximately the same or statistically superior improvement in time to ≥1 mm ST-segment depression as compared to administration of a similar amount of nicorandil compound(s) by twice-daily administration of an immediate release formulation (e.g., BID administration of Ikorel tablets) at 6, 8, 12, 14, or 18 hours after administration (aspect 21).

Another case is a pharmaceutically acceptable composition comprising a therapeutically effective amount of one or more nicorandil compounds that are therapeutically equivalent or superior to nicorandil with respect to one or more nicorandil-treatable conditions (e.g., one or more forms of angina) and a controlled release formulation wherein once daily administration of the composition results in delaying the time to ≥1 mm ST-segment depression by about 20% to 50% from baseline at 6, 8, 12, 14, or 18 hours after administration (aspect 22).

Still further provided is a pharmaceutically acceptable composition comprising a therapeutically effective amount of one or more nicorandil compounds that are therapeutically equivalent or superior to nicorandil with respect to one or more nicorandil-treatable conditions (e.g., one or more forms of angina) and a controlled release formulation, wherein upon administration to human patients the composition contains significantly more nicorandil compound(s) than the amount of nicorandil compound(s) retained in the Nikoran formulation 6 hours after administration to a human patient (aspect 23).

Further embodiments comprise a pharmaceutically acceptable composition comprising a therapeutically effective amount of one or more nicorandil compounds that are therapeutically equivalent or superior to nicorandil with respect to one or more nicorandil-treatable conditions (e.g., one or more forms of angina) and a controlled release formulation, wherein once-daily administration of the composition to a human patient results in (a) a statistically significantly greater amount of nicorandil being delivered, to one or more parts of the intestine (e.g., the jejunum) from the composition than are delivered from one or both of (1) once daily administration of a similar amount of nicorandil compound(s) from the Nikoran formulation and (2) twice-daily administration of a similar amount of nicorandil compound(s) from the Ikorel formulation, (b) at least 50% of the administered dose of nicorandil being delivered to the intestine, or (c) both (a) and (b) (aspect 24).

An aspect of the invention is a pharmaceutically acceptable composition comprising a therapeutically effective amount of one or more nicorandil compounds contained in a plurality of a first controlled release dosage forms comprising a first controlled release formulation and a plurality of second controlled release dosage forms comprising a second controlled release formulation, wherein the first controlled release formulation and second controlled release formulation release significantly different amounts of nicorandil compound(s) in different parts of the gastrointestinal tract of recipient patients, at different periods of time after administration to patients, or both, wherein the first controlled release dosage forms and second controlled release dosage forms are contained in a single delivery facilitating component of the composition, wherein the composition is at least about as therapeutically effective or is statically significantly more therapeutically effective than a twice daily immediate release formulation of the same amount of the nicorandil compound(s) (e.g., the compound(s) in the Ikorel formulation) in the treatment of one or more nicorandil-treatable conditions or the modulation of one or more nicorandil-modifiable physiological conditions (aspect 25).

Embodiments comprise a pharmaceutically acceptable composition comprising a therapeutically effective amount of one or more nicorandil compounds in a controlled release formulation dosage form that (1) when contacted with a first solution (or, e.g., condition) having a pH of 1-1.5 (a) releases a total of about 15-30% of the therapeutically effective amount after about 0.75-1.5 hours of contact with the first solution, (b) releases a total of about 20-40% of the therapeutically effective amount after about 1.5-2.5 hours of contact with the first solution, and (c) releases a total about 25-50% of the therapeutically effective amount after about 2.5-5 hours of contact with the first solution, and (2) after contacting the composition with the first solution (or, e.g., condition) for a total of 2.5-5 hours and thereafter further contacting the composition with a second solution (or, e.g., condition) having a pH of 5.5-7 (a) releases a total of about 55-70% of the therapeutically effective amount after about 1 hour of contact with the second solution, (b) releases a total of about 65-85% of the therapeutically effective amount after about 2 hours of contact with the second solution, (c) releases a total of about 80-90% of the therapeutically effective amount after about 6 hours of contact with the second solution, and (d) after at least about 8 hours of contact with the second solution, releases a total of at least about 90% of the therapeutically effective amount of the nicorandil compound(s) (aspect 26).

Aspects include a pharmaceutically acceptable composition comprising a therapeutically effective amount of one or more nicorandil compounds in a controlled release formulation dosage form that (1) releases about 15-25% of the therapeutically effective amount when the composition is contacted with a first solution having a pH of 1-1.5 for 0.75-1.5 hours, (2) thereafter releases an additional detectable amount of nicorandil such that about 20-40% of the therapeutically effective amount is released from the composition when the composition is contacted with the first solution for a total of 1.5-2.5 hours, (3) thereafter releases an additional detectable amount of nicorandil such that about 35-50% of the therapeutically effective amount is released from the composition when the composition is contacted with the first solution for a total of 2.5-4.5 hours, (4) thereafter releases an additional detectable amount of nicorandil such that about 40-60% of the therapeutically effective amount is released from the composition after contacting the composition with a second solution having a pH of 5.5-7 for 1.5-2.5 hours, (5) thereafter releases an additional detectable amount of nicorandil such that about 65-85% of the therapeutically effective amount is released from the composition after contacting the composition with the second solution for a total of 3-4 hours, and (6) thereafter releases an additional detectable amount of nicorandil such that at least about 90% of the therapeutically effective amount is released after contacting the composition with the second solution for a total of 8-12 hours (aspect 27).

An aspect provides a pharmaceutically acceptable composition comprising a therapeutically effective amount of one or more nicorandil compounds in a controlled release formulation dosage form that (1) releases about 15-30% of the therapeutically effective amount at about one hour after administration, (2) thereafter releases an additional detectable amount of the one or more nicorandil compounds such that about 20-40% of the therapeutically effective amount is released from the composition at about two hours after administration, (3) thereafter releases an additional detectable amount of the one or more nicorandil compounds such that about 35-50% of the therapeutically effective amount is released from the composition at about four hours after administration (4) thereafter releases an additional detectable amount of the one or more nicorandil compounds such that about 45-65% of the therapeutically effective amount is released from the composition at about six hours after administration, (5) thereafter releases an additional detectable amount of the one or more nicorandil compounds such that about 55-75% of the therapeutically effective amount is released from the composition at about seven hours after administration, (6) thereafter releases an additional detectable amount of the one or more nicorandil compounds such that about 65-85% of the therapeutically effective amount is released from the composition at about eight hours after administration, and (7) thereafter releases an additional detectable amount of the one or more nicorandil compounds such that at least about 90% of the therapeutically effective amount is released about 16 hours after administration (aspect 28).

Yet another facet is a pharmaceutically acceptable composition comprising a therapeutically effective amount of one or more nicorandil compounds in a controlled release formulation that detectably releases the one or more nicorandil compounds from the controlled release formulation dosage form over a release period of at least about 12, 14, or 16 hours from administration to a human patient, wherein (1) the release of the one or more nicorandil products occurs at a first release rate for a first period of about 1.5-2.5 hours, a second release rate for a second period of about 3.5-about 4.5 hours, a third release rate for a third period of about 3-about 4 hours, and a fourth release rate for a fourth period of about 6-about 7 hours, (2) about 50% of the one or more nicorandil compounds is released from the controlled release dosage form during the period consisting of the first period and second period, (3) the rate of release of the one or more nicorandil compounds during the first period being about 1.25× to 1.75× the release rate of the one or more nicorandil compounds during the second period (or the amount being released in this period being about 1.25× to 1.75× that of, or 25%-75% greater than, that released during the second period), (4) at least about 40% of the one or more nicorandil compounds is released from the controlled release formulation dosage form during the period consisting of the third period and the fourth period, and (5) the rate of release of the one or more nicorandil compounds during the third period being about 1.25× to about 1.75× the release rate of the one or more nicorandil compounds during the fourth period (or the amount released during the third period being 25%-75% greater than the amount of the one or more nicorandil compounds released during the fourth period) (aspect 29).

Still further provided is a pharmaceutically acceptable composition comprising a therapeutically effective amount of one or more nicorandil compounds in a controlled release formulation comprising (1) a first release component comprising a first amount of one or more nicorandil compounds and (2) a second release component comprising a second amount of one or more nicorandil compounds and a pH-sensitive release controlling agent, (3) the first and second amount collectively comprising a therapeutically effective daily dose of the one or more nicorandil compounds, (4) wherein contacting the second release component and the first release component with a solution having a pH of about 1-1.5 under moderate stirring (under conditions that result in a substantially similar dissolution to the use of a USP apparatus type II system (Agilent) at 50 rpm) for about 2 hours results in (a) about 40%-about 80% of the first amount (e.g., first effective amount) being released from the first release component and (b) no more than about 15% of the second amount (e.g., second effective amount) being released from the second release component, and (5) wherein contacting the second release component with a solution having a pH of 6-7 at about room temperature and under moderate stirring for about 2 hours results in about 20%-about 60% of the second amount being released from the second release component (aspect 30).

Also provided is a pharmaceutically acceptable composition comprising a therapeutically effective amount of one or more nicorandil compounds in a controlled release formulation comprising (1) between 3-6 solid dosage forms collectively comprising a therapeutically effective amount of one or more nicorandil compounds, at least some of the solid dosage forms (e.g., ≥33%, ˜50%, or ˜65% of the solid dosage forms) comprising a first release formulation and the remainder of the solid dosage forms comprising a second release formulation, wherein the second release formulation releases significantly less of the one or more nicorandil compounds therein in the stomach than the first release formulation solid dosage forms under one or more pH conditions (e.g., at pH 1.2), and (2) a pharmaceutically acceptable delivery facilitating casing, such as a capsule, that surrounds the solid dosage forms (aspect 31).

An aspect comprises a pharmaceutically acceptable composition comprising a therapeutically effective amount of one or more nicorandil compounds in a controlled release formulation that releases the one or more nicorandil compounds at a rate such that the composition is therapeutically effective in treating one or more nicorandil-treatable conditions in a significant number of patients when administered in a once-daily regimen (aspect 32).

Further provided is a pharmaceutically acceptable composition comprising a therapeutically effective amount of one or more nicorandil compounds in a controlled release formulation comprising 3-8, such as 3-6 dosage form units, at least 33% of which dosage form units comprising an enteric dissolution coating which is dissolved on average between about 2-7 or 2-6, such as about 4-6, such as about 4.5-6.5 hours after administration of the composition to a human patient, and thereafter releasing at least about 75% of the nicorandil compound(s) contained therein, the remainder of the dosage form units releasing at least about 80% of the one or more nicorandil compounds contained therein on average within about 4.5 hours of administration of the composition to a human patient (aspect 33).

Embodiments include a pharmaceutically acceptable composition comprising a therapeutically effective amount of one or more nicorandil compounds contained in a first release formulation and a second release formulation, the first release formulation and second release formulation each comprising pH independent/insensitive means for controlled release of the nicorandil compound(s), means for binding the nicorandil compound(s), and means for promoting disintegration of the first release formulation and the second release formulation further comprising means for preventing dissolution of the second release formulation in gastric acid pH conditions (e.g., at a pH of 1.2) (aspect 34).

Facets comprise a pharmaceutically acceptable composition comprising a therapeutically effective amount of one or more nicorandil compounds in a controlled release formulation produced by a process comprising forming a collection of e.g., 2-8, 3-8, or 3-6 solid dosage forms, each of the solid dosage forms being produced by a method comprising (1) forming a first mixture comprising (a) a therapeutically effective amount of one or more nicorandil compounds (e.g., about 5-20 mg, such as about 5-15 mg, or about 7.5-12.5 mg nicorandil), (b) about 25-45 mg of a controlled release pH-independent matrix polymer component (e.g., hydroxypropyl methylcellulose (HPMC)), (c) about 50-120 mg of one or more binder components (e.g., composed of a disintegrant binder and a highly compressible binder, such as being composed about 30-60 mg of a microcrystalline cellulose, such as a SMCC and about 0-35, ˜5-35, ˜0-30, ˜5-25-5-30, ˜10-40, ˜10-35, ˜10-30, ˜10-25, ˜15-30, ˜15-25 or about 20 mg starch), and mixing these ingredients for a suitable time (e.g., at least about 15 minutes) under suitable conditions (e.g., using a mixer operating at a speed of about 15 rpm to about 35 rpm (e.g., in, or in a system significantly similar in performance to mixing in a Turbula mixer)), (2) adding about 0.25-1.5 mg of magnesium stearate to such first mixture and further mixing for at least about 5 minutes under suitable mixing conditions (e.g., at a speed of about 15 rpm to 35 rpm) to form a second mixture, and (3) compressing the second mixture to form a solid dosage form with a hardness of at least about 60 N, and (4) optionally coating at least 33% of the solid dosage forms produced by the method with a coating solution comprising about 5-7.5 mg of a pH sensitive controlled release polymer component (e.g., a composition composed mostly of a methylacrylate polymer) (aspect 35).

Further aspects provide a composition of (according to) any one or more of aspects 2-35, wherein the nicorandil compounds(s) are contained in a gastric acid dissolution susceptible component (GADSC) and a gastric acid dissolution resistant component (GADRC), wherein upon maintaining the composition in contact with a pH 1.2 dissolution media for a period of about 2 hours (1) the GADRC releases a statistically significantly smaller proportion of the one or more nicorandil compounds in the GADRC than the proportion of one or more nicorandil compounds released from the GADSC and (2) the composition releases no more than 50% of the one or more nicorandil compounds contained in the composition (aspect 36).

Aspects include a composition according to aspect 1 or aspect 36, wherein the GADRC releases no more than 20% of the one or more nicorandil compounds contained in the GADRC at 2 hours of contact with a dissolution media having a pH of 1.2 (such as releasing no more than ˜15%, no more than ˜12.5%, no more than ˜10%, no more than ˜7.5%, or no more than ˜5%, e.g. about ˜1-20%, ˜1-15%, ˜1-10%, ˜1-5%, ˜2-20%, ˜2-15%, ˜2-10%, ˜2-5%, ˜3-15%, ˜3-12%, ˜5-20%, ˜5-15%, or ˜5-10% of the nicorandil compound(s) initially contained in the GADRC under such condition(s) (aspect 37).

Embodiments include a composition of any one or more of aspect 1, aspect 36, or aspect 37, wherein the GADSC comprises a controlled release formulation comprising an amount of one or more release retardant/resistant polymers that permits the release of at least 33% and the retention of at least about 2.5%-10%, e.g., about 3-7%, e.g., about 5%, of the one or more nicorandil compounds initially present in GADSC 5 hours or 6 hours after contact with a dissolution media having a pH of about 1.2 or a dissolution media having a pH of about 6.8 (aspect 38).

Embodiments include a composition of any one or more of aspects 1 and 36-38, wherein the GADRC comprises an amount of one or more pH responsive polymers that is effective to release at least 33% of the one or more nicorandil compounds initially present in the pH responsive dosage form 5 hours or 6 hours after contact with a dissolution media having a pH of about 6.8 and no more than 20% of the one or more nicorandil compounds initially present in the pH responsive dosage form 5 hours or 6 hours after contact with a dissolution media having a pH of about 1.2 (aspect 39).

A facet is a composition of any one or more of aspects 1 and 36-39, wherein the GADSC comprises a first portion of the therapeutically effective amount of the one or more nicorandil compounds and the GADRC comprises a second portion of the therapeutically effective amount of the one or more nicorandil compounds, wherein the first portion comprises 30-70% of the one or more nicorandil compounds in the composition (aspect 40).

Embodiments include a composition of aspect 40, wherein the first portion of the therapeutically effective amount of the one or more nicorandil compounds and the second portion of the therapeutically effective amount of the one or more nicorandil compounds are equal or differ in amount by less than about 10% (aspect 41).

Embodiments also include a composition of any one or more of aspects 1-41, wherein the therapeutically effective amount of the one or more nicorandil compounds in the composition is an amount demonstrated to provide a therapeutically effective once daily dosage of nicorandil in a significant number of patients, in most patients, or both (e.g., as determined by one or more well-controlled and adequate clinical studies, such as, e.g., a study recognized by a regulatory authority such as the United States Food and Drug Administration or, e.g., an equivalent thereof) (aspect 42).

An additional embodiment is a composition of any one or more of aspects 1-42, wherein most, generally all, substantially all, or all of the one or more nicorandil compounds is nicorandil (aspect 43).

Embodiments further include a composition according to aspect 43, wherein the therapeutically effective amount is 10-100 mg nicorandil (e.g., about or precisely 10-80, 10-75, 10-60, 10-50, 12.5-75, 12.5-60, 15-75, 15-60, 17.5-70, 20-60, 20-50, or about/precisely 20-40 mg of nicorandil, such as about or precisely 10, 12.5, 15, 20, 22.5, 25, 27.5, 30, 33, 35, 37.5, 40, 42.5, 45, 50, 55, or 60 mg of nicorandil) (aspect 44).

Another embodiment is a composition of any one or more of aspects 1 and 36-44, wherein the GADSC comprises two or more GADSC dosage forms, the GADRC comprises two or more GADRC dosage forms, or both (aspect 45).

An exemplary embodiment is a composition of aspect 45, wherein the composition comprises at least 3 dosage forms made up of 1-5 or 2-5 GADSC dosage forms and 1-5 or 2-5 GADRC dosage forms (aspect 46).

A particular embodiment is a composition of aspect 46, wherein the composition comprises 2-5 GADSC dosage forms and 2-5 GADRC dosage forms (aspect 47).

Embodiments include a composition of any one or more of aspects 1 and 36-47, wherein the GADSC dosage forms are uncoated controlled release matrix tablets and the GADRC dosage forms are coated controlled release matrix tablets, wherein the coating of the GADRC dosage forms comprises an effective amount of one or more pH responsive polymers (aspect 48).

Further provided is a composition of any one or more of aspects 1 and 36-48, wherein the GADSC, GADRC, or both the GADSC and GADRC, comprises a matrix formulation comprising (1) about 25-45% or 30-40% of a release retardant polymer component and (2) about 40-70%, such as about 45-65% or ˜50-60% of a binder/disintegrant component (aspect 49).

Embodiments of the invention herein include a composition of any one or more of aspects 1 and 36-49, wherein the GADSC, GADRC, or both the GADSC and GADRC, comprises a matrix formulation comprising (1) a release retardant polymer component, wherein the release retardant polymer component and one or more nicorandil compounds are present in a ratio of about 5:1 to about 2:1, such as about 4.5:1 to about 2.5:1, about 4:1 to about 3:1, or about 3.5:1 or (and/or) (2) a binder/disintegrant component, wherein the binder/disintegrant component and one or more nicorandil compounds are present in a ratio of ˜4:1 to 7:1, or 4.5:1 to 6.5:1, or 5:1 to 6:1, or about 5.5:1 (aspect 50).

A facet is a composition of any one or more of aspects 1 and 36-50, wherein at least about 80%, such as at least about 85%, 90%, or 95% of GADSC, GADRC, or both, is composed of (1) the one or more nicorandil compounds, (2) a binder/disintegrant component, and (3) a release retardant polymer component (aspect 51).

Another aspect is a composition of any one or more of aspects 1 and 36-51, wherein at least about 20% of the composition of the GADSC, GADRC, or both, is/are composed of a microcrystalline cellulose composition, such as a mixture of a silicified microcrystalline cellulose and colloidal silicon dioxide (aspect 52).

Further provided is a composition of any one or more of aspects 1-52, wherein most, generally all, substantially all, or all of any pH-independent release resistant polymer composition in the composition (e.g., in a GADSC, GADRC, or both), is composed of hydroxyl methylcellulose (HPMC) (aspect 53).

Additionally provided is a composition of any one or more of aspects 1-53 wherein most, generally all, substantially all, or all of the excipients (individually) (i.e., most, generally all or each of the excipients) in the composition (e.g., in a GADSC, GADRC, or both), comprise (per excipient) less than 6% moisture content, such as less than 5, 4, or 3% moisture content (aspect 54).

Still further provided is a composition (in this case, formulation and packaging) according to any one or more of aspects 1-54 in further combination with an effective amount of one or more pharmaceutically acceptable wicking agents/desiccants that detectably or significantly reduce moisture in the composition (aspect 55).

A more particular aspect is a composition (formulation and packaging) of aspect 55, wherein the desiccant is at least mostly composed of silica (aspect 56).

In a further aspect, provided is a composition (formulation and packaging) of aspect 56, wherein the composition is contained in a unit dosage form aluminum-aluminum blister package comprising a silica desiccant layer (aspect 57).

Also provided is a composition of any one or more of aspects 1-57, wherein the composition exhibits at least about 95% stability (retention) of the initial one or more nicorandil compounds of the composition after storage of at least 1, 3, 6, 12, or 24 months at ˜5° C., ˜25° C. and ˜60% RH, or both (aspect 58).

An aspect of the invention is a composition of any one or more of aspects 1 and 36-58, wherein the GADSC, GADRC, or both, are mostly composed of one or more tablets, wherein each of the one or more tablets has a hardness of at least about 60 N, at least about 95 N, such as at least about 120 N (e.g., 90-150 N, 100-140 N, 100-130 N, etc.) (aspect 59).

A facet is a composition of any one or more of aspects 1 and 36-59, wherein the GADRC comprises a formulation that exhibits a controlled release when the GADRC is contacted with a dissolution media having a pH of 6.8, such that at least about 50% of the one or more nicorandil compounds initially present in the GADRC are retained after the GADRC contacts the dissolution media for 2 hours and at least about 10% (e.g., ≥˜12.5% or ≥˜15%) of the one or more nicorandil compounds initially present in the GADRC are present after the GADRC contacts the dissolution media for 8 hours (aspect 60).

Also provided is a composition of any one or more of aspects 1-60, wherein the composition when initially contacted with a dissolution media of pH 1.2 for about 6 hours is associated with at least about 15%, such as at least about 20%, or at least about 25% less of one or more of imp A, imp B, imp C, imp D than Nikoran OD tablets (aspect 61).

An aspect of the invention is a composition of any one or more of aspects 1 and 36-61, wherein when maintained in contact with a dissolution media of pH 6.8, the amount of the one or more nicorandil compounds released from the GADSC is mostly, generally, or entirely in accordance with the following release profile: (1) about 0.5-25%, e.g., about 5-20%, about 5-15%, or about 12% of the nicorandil compound(s) being released from the GADSC after 0.25 hours in the dissolution media; (2) about 11.5% to about 44.5%, such as about 20% to about 35%, such as about 28% of the nicorandil compound(s) (NCDC(s)) released after 1 hour in the dissolution media; (3) about 25% to about 60%, such as about 32% to about 52%, such as about 42.5% of the NCDC(s) released at 2 hours in the dissolution media; (4) about 60-92.5%, e.g., about 65-85%, such as about 75% or about 76% of the NCDC(s) released at 6 hours in the dissolution media; (5) about 71% to about 98%, such as about 80% to about 90%, such as about 84.5% of the NCDC(s) released at 8 hours contact with the dissolution media, and (6) about 83% to about 100%, such as about 87% to about 97%, e.g., about 92.25% of the NCDC(s) in the GADSC being released at 14 hours of contact with the dissolution media (aspect 62).

Another aspect is a composition of any one or more of aspects 1 and 36-62, wherein when maintained in contact with a dissolution media of pH 6.8 the amount of the one or more nicorandil compounds released from the GADRC is in accordance mostly, generally, or entirely with the following release profile: (1) about 0% to about 8.5%, such as about 3% to about 8%, such as about 7% of the NCDC(s) being released from the GADRC after 0.25 hours in the dissolution media; (2) about 15.5% to about 29%, such as about 19% to about 25%, such as about 22.25% of the NCDC(s)) being released at 1 hour in the dissolution media; (3) about 27% to about 50%, such as about 33% to about 43%, such as about 38.3% of the NCDC(s) being released at 2 hours in the dissolution media; (4) about 65-80%, e.g., about 67-77%, such as about 72.5% of the NCDC(s) being released at 6 hours in the dissolution media; (5) about 75% to about 87.5%, such as 78% to about 85%, such as about 81.5% of the NCDC(s) being released at 8 hours contact with the dissolution media, and (6) about 83.5% to about 98% or about 100%, such as about 87% to about 94%, e.g., about 90.5% of the NCDC(s) in the GADSC being released at 14 hours of contact with the dissolution media (aspect 63).

Embodiments include a composition of any one or more of aspects 1 and 36-63, wherein when maintained in contact with a dissolution media of pH 1.2 the amount of the one or more nicorandil compounds being released from the GADSC is in accordance mostly, generally, or entirely with the following release profile: (1) about 11% to about 28%, such as about 15% to about 23%, such as about 19.5% of the NCDC(s) being released from the GADSC after 0.25 hours in the dissolution media; (2) about 35% to about 50%, such as about 38% to about 48%, such as about 43.25% of the NCDC(s)) in the GADSC being released at 1 hour in the dissolution media; (3) about 55% to about 70%, such as about 60% to about 65%, such as about 62.5% of the NCDC(s) being released at 2 hours in the dissolution media; and (4) about 80-about 100%, e.g., about 95-99%, such as about 97% of the NCDC(s) being released at 6 hours in the dissolution media; (aspect 64).

Provided is a composition of any one or more of aspects 1 and 36-64, wherein the composition is a pharmaceutically acceptable capsule comprising a GADRC and a GADSC, wherein when the capsule is maintained in contact with a dissolution media of pH 6.8 the amount of the one or more nicorandil compounds released from the capsule is in accordance mostly, generally, or entirely with the following release profile: (1) about 0-11%, such as about 2% to about 5%, such as about 3.5% of the NCDC(s) being released from the capsule at/after 0.25 hours in the dissolution media; (2) about 10% to about 25%, such as about 15% to about 20%, such as about 17.5% of the NCDC(s) in the capsule being released at 1 hour in the dissolution media; (3) about 25% to about 35%, such as about 27.5% to about 32.5%, such as about 30% of the NCDC(s) being released at 2 hours in the dissolution media; (4) about 53-about 61%, e.g., about 55-59%, such as about 57% of the NCDC(s) being released at 6 hours in the dissolution media; (5) about 65%-76%, such as about 67% to about 74%, such as about 70.5% of the NCDC(s) being released at/after 8 hours, (6) about 71.5% to about 83.5%, such as 75%-81%, such as about 77.5% of the NCDC(s) being released at 10 hours in the media; (7) about 76% to about 90%, such as about 80-86%, such as about 83% of the NCDC(s) being released at 12 hours; (8) about 81.5% to about 92%, such as about 85-89%, such as about 86.75% of the NCDC(s) being released at 14 hours; and (9) about 85% to about 95% or 100%, such as about 88-92%, such as about 90% of the NCDC(s) being released at 18 hours (aspect 65).

Embodiments herein include a composition of any one or more of aspects 1 and 36-65, wherein the composition is a pharmaceutically acceptable capsule comprising a GADRC and a GADSC, wherein the capsule when maintained in contact with a dissolution media of pH 1.2 the amount of the one or more nicorandil compounds released from the capsule is in accordance mostly, generally, or entirely with the following release profile: (1) about 3.5% to about 12%, such as about 5% to about 10%, such as about 7.75% of the NCDC(s) being released from the capsule at/after 0.25 hours in the dissolution media; (2) about 13% to about 21%, such as about 15% to about 19%, such as about 17% of the NCDC(s) in the capsule being released at 1 hour in the dissolution media; (3) about 22% to about 28%, such as about 23% to about 27%, such as about 25% of the NCDC(s) being released at 2 hours in the dissolution media; (4) about 35.5%-about 47.5%, e.g., about 39-44%, such as about 41.5% of the NCDC(s) being released at 6 hours in the dissolution media; (5) about 40.5%-50.5%, such as about 43% to about 48%, such as about 45.5% of the NCDC(s) being released at/after 8 hours, (6) about 41% to about 55%, such as 46%-50%, such as about 48% of the NCDC(s) being released at 10 hours in the media; (7) about 45.5% to about 55.5%, such as about 49%-52%, such as about 50.5% of the NCDC(s) being released at 12 hours; (8) about 48.5% to about 58.5%, such as about 50% to about 57%, such as about 53.5% of the NCDC(s) being released at 14 hours; and (9) about 55% to about 65% such as about 57.5%-62.5%, such as about 60% of the NCDC(s) being released at 18 hours in the dissolution media (aspect 66).

Embodiments herein include a composition of any one or more of aspects 1-66, wherein the composition when administered to a human patient exhibits most, generally all, or all of the following release profile characteristics: (1) about 32-46% (e.g., 36-42%, such as about 39%) of the NCDC(s) initially in the composition remaining available for release (i.e., in the composition) 1 hour post administration to a human patient; (2) about 19.5%-42.5%, e.g., about 26-36%, such as about 31% of the NCDC(s) of the composition remaining available for release at about (after) 2 hours post administration; (3) about 12% to about 15.5%, such as about 13% to about 14%, such as about 13.5% or 13.75% of the NCDC(s) of the composition remaining available for release 6 hours post administration; (4) about 3.5% to about 15.%, such as about 7.5% to about 11.5%, e.g., about 9.5% of the NCDC(s) of the composition remaining available for release at 8 hours post administration; and (5) about 2.5% to about 8%, such as about 3.5% to about 6.5%, such as about 5% of the NCDC(s) of the composition remaining available for release at 12 hours post administration to the human patient (aspect 67).

Another aspect of the invention includes a composition of any one or more of aspects 1 and aspects 36-67, wherein the GADSC and GADRC are co-administered in a delivery facilitating component (aspect 68).

Embodiments herein include a composition of aspect 68, wherein the composition comprises multiple GADSC dosage forms and multiple GADRC dosage forms contained in a delivery facilitating component (aspect 69).

Embodiments herein include a composition of aspect 69, wherein the GADSC dosage forms are tablets, the GADRC dosage forms are tablets, and the delivery facilitating component is a pharmaceutically acceptable capsule (aspect 70).

Further provided by the invention herein is a composition according to any one or more of aspects 1-70 wherein most, generally all, or all of the dosage forms of the composition comprise (1) a core comprising a highly compactible pharmaceutically acceptable binder, a pharmaceutically acceptable disintegrant binder, and a pharmaceutically acceptable controlled release polymer, wherein the highly compatible binder, disintegrant binder, and controlled release polymer are present in a ratio of about 1-2:about 0.2-1:about 0.8-1.8 and (2) a coating comprising a pH-resistant release controlling polymer and a plasticizer, wherein the pH-release resistant polymer and plasticizer are present in a ratio of about 5:1 to about 7:1 (aspect 71).

Also provided in embodiment(s) is a composition according to any one or more of aspects 1 and 36-71, wherein most, generally all, or all of the GADRC dosage forms of the composition comprise (1) a core comprising about 25-50% of a highly compactible binder, about 5-25% of a disintegrant binder, and about 20-45% of a cellulose ether derivative polymer and (2) a coating comprising an effective amount of a pH-resistant release controlling polymer and an effective amount of a pharmaceutically acceptable plasticizer, wherein the pH-release resistant polymer constitutes at least about 65% of the coating (aspect 72).

An aspect is a composition according to any one or more of aspects 1 and 36-72, wherein most, generally all, or all GADSC dosage forms in the composition when maintained in pH 6.5-7 dissolution media (e.g., pH 6.8 dissolution media) (1) releases about 30%-about 70% of the NCDC(s) in the GADSC within two hours, and (2) releases no more than 15%-25% of the NCDC(s) in the GADRC at about two hours at pH of about 1.2 (aspect 73).

Embodiments include a composition according to any one or more of aspects 1-73, wherein the composition is contained in a package of a plurality of units of a pharmaceutically acceptable composition, each unit contained in an individual single container with an approximately uniform amount of a pharmaceutically acceptable desiccant, wherein the proper opening of any of the units does not significantly impact the stability of the one or more nicorandil compounds contained in any of the other units in the package (aspect 74).

A facet of the invention includes a composition according to any one or more of aspects 1-74, wherein the NCDC(s) make up about 7.5 wt. % to about 12.5 wt. % of the composition, (2) about 25-60% (wt. %) of the composition is silicified microcrystalline cellulose (3) about 20-45% of the composition is hydroxypropyl methylcellulose (HPMC), about 2%-6% of the composition is composed of a methacrylic acid-ethyl acrylate copolymer, and up to about 25% of the composition is composed of starch (aspect 75).

Embodiments herein include a composition of aspect 75, wherein the composition further comprises about 0.1%-1% triethyl citrate (aspect 76).

An aspect of the invention includes a composition according to any one or more of aspects 1-76, wherein at least 33%, 40%, 50% (e.g., about 33%-66% or ˜45-55%) of the dosage form(s) in the composition are coated with a coating comprising about 55-99 wt. % of a methacrylic acid-ethyl acrylate copolymer (aspect 77).

An aspect herein includes a composition according to any one or more of aspects 1-77, wherein the composition comprises at least 2 solid dosage forms, such as 3-8 or 3-6 solid dosage forms, wherein most, generally all, or all of the dosage forms are tablets having a maximum dimension (e.g., diameter, length, height/thickness, or a combination thereof) of about 2.7-8 mm, such as 3.5-7.5 mm, e.g., 4-7 mm or about 5 mm (aspect 78).

Aspects of the invention include a composition according to aspect 78, wherein the solid dosage forms of the composition are contained in a size 0 (zero), OEL (zero elongated) or 00 (zero, zero) capsule, which optionally is composed of HPMC, gelatin, or a mixture thereof (aspect 79).

Embodiments herein include a composition of any one or more of aspects 1-79, wherein the composition comprises an effective amount of mean(s) for maintaining ingredient flowability, mean(s) for coating plasticization, or both (aspect 80).

An embodiment herein is a composition of any one or more of aspects 1-80, wherein (1) at least one component of the composition is associated with a Cmax that is significantly less than the Cmax of a corresponding formulation comprising the same amount of the one or more nicorandil compounds in a formulation corresponding to the IKOREL®10 mg formulation or (and/or) (2) the Tmax of the composition is significantly greater than the Tmax of the NCDC(s) in a formulation corresponding to the IKOREL®10 mg formulation, wherein the composition detectably releases nicorandil for a period of at least about 12 or 14 hours post administration to a human patient (aspect 81).

Further provided herein is a composition according to any one or more of aspects 1-81, wherein the composition when administered once daily to an adequately powered population of human subjects, results in significantly less of one or more types of nicorandil-associated adverse events as compared to human patients that receive twice-daily administration of a corresponding amount of the one or more nicorandil compounds in the IKOREL®10 mg formulation (aspect 82).

An embodiment herein is a composition according to any one or more of aspects 1-82, wherein the composition about 2-6 wt. % of a pH sensitive second release polymer component that coats at least part of a core composition of one or more dosage forms in the composition, wherein the core composition comprises (a) at least some portion of the therapeutically effective amount of the one or more nicorandil compounds, (b) about 25-45 wt. % of a controlled release polymer component that is in mixture with the therapeutically effective amount of the one or more nicorandil compounds in a core composition, (c) about 25-60% of a compaction and flowability promoting agent in mixture with the one or more nicorandil compounds and the controlled release polymer component in the core composition (and which optionally has an average particle size of about 100-150 μm, a bulk density of about 0.35-0.55 g/mL, or both, and which optionally further is significantly dispersible but not significantly dissolvable in water), and (d) about 0.1% to about 1% of a pharmaceutically acceptable coating plasticizer (aspect 83).

Also provided in embodiment(s) is a composition of any one or more of aspects 1-83, wherein the composition comprises, mostly comprises, generally only comprises, nicorandil compound-containing dosage forms (1) having a hardness of about 70-130 Newtons (N), such as about 70-120 N, such as about 90-130 N or about 90-120 N or about 110 N or (and/or) (2) a mass of about 90-120 mg, wherein optionally the nicorandil dosage forms are contained in a vehicle/delivery agent, such as a pharmaceutically acceptable capsule (aspect 84).

Still further provided is a composition of any one or more of aspects 1-84, wherein the composition is free of any detectable or significant amount of ethyl cellulose (e.g., contains less than 0.5%, less than 0.2%, less than 0.1%, less than 0.01%, less than 0.001%, or less than 0.00001% ethyl cellulose) (aspect 85).

Embodiments herein include a composition of any one or more of aspects 1-85, wherein the composition comprises one or more pH sensitive polymers that coat at least some of the nicorandil-associated dosage form in the composition, wherein the pH sensitive polymers mostly, generally, or only comprise one or more polymethacrylate-based copolymers, hydroxypropyl methylcellulose (HPMC), acrylic acid polymers, or a combination of some or all thereof (aspect 86).

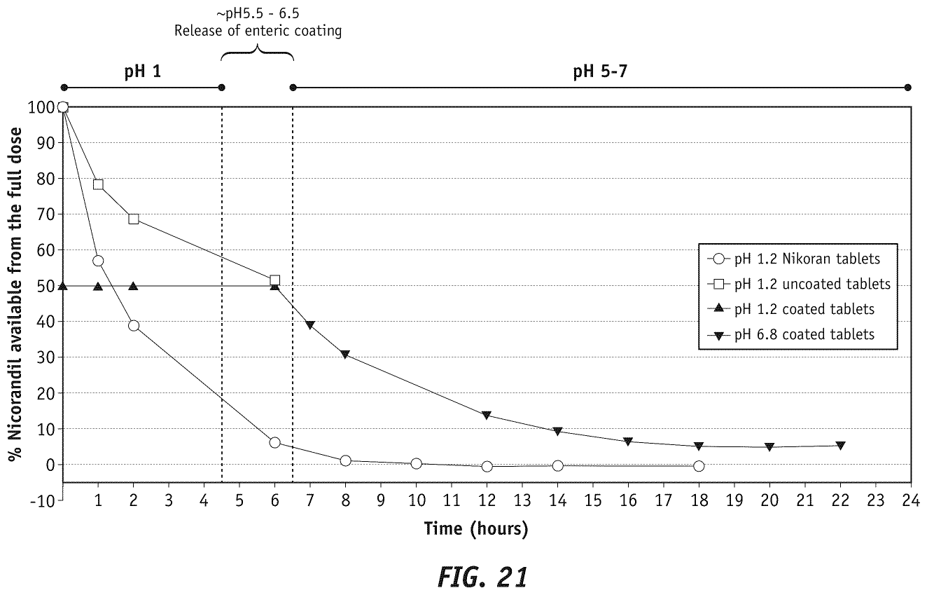

Embodiments herein include a composition of any one or more of aspects 1-86, wherein the composition is produced by a process comprising generally all or all of the steps presented in FIG. 21 (aspect 87).

Further provided herein is a composition of aspect 87, wherein the composition is produced by a process comprising mixing about 5-7.5 mg of one or more pH sensitive polymers with about 0.1 to 1 mg of a plasticizer component for a period of at least about 15 minutes and contacting at least some of the solid dosage forms of the composition with the coating solution for a period of at least about 15 minutes (aspect 88).

An embodiment of the invention provides a composition of aspect 87 or 88, wherein solid dosage forms in the composition are produced by a method that comprise a compaction step that comprises applying dry granulation, direct compression, or both, of the solid dosage form material (e.g., to form tablet(s)) (aspect 89).

The invention further provides a method of modulating adenosine triphosphate-sensitive potassium (KATP) channels in a mammalian host comprising delivering an effective amount of a composition according to any one or more of aspects 1-90 to the host (aspect 90).

Provided also is a method of increasing blood vessel diameter in a mammalian host comprising delivering an effective amount of a composition according to any one or more of aspects 1-90 to the host (aspect 91).

Additionally provided is a method of increasing availability of nitric oxide (NO) in the circulation of a mammalian host comprising administering an effective amount of a composition according to any one or more of aspects 1-90 to the host (aspect 92).

An embodiment further includes a method of causing significant dilation of coronary arteries, a significant reduction of myocardial oxygen demand, or both, in a mammalian host comprising administering an effective amount of a composition according to any one or more of aspects 1-90 to the host (aspect 93).

An additional aspect is a method of any one of aspects 1-93, wherein the host is a human (aspect 94).

An embodiment is a method of any one or more of aspects 1-94, wherein the composition is administered to the host no more than once per day during some, most, generally all, or all of the period that the host is treated with the composition (aspect 95).

Further provided is a method of treating one or more forms of angina, causes of angina, or symptoms of angina in a human patient, comprises administering to the patient an effective amount of a composition according to any one or more of aspects 1-90 for a treatment period, wherein during most, generally all, or all of the treatment period the composition is administered to the patient no more than once per day (aspect 96).

An aspect is a method of aspect 97, wherein the angina treated by the method comprises chronic stable angina pectoris (CSAP).

Further provided is the use of a composition according to any one of the preceding composition aspects as a medicament or in the preparation of a medicament for the treatment of (e.g., for use in the treatment of) one or more nicorandil-treatable conditions (aspect 98).

A final exemplary aspect provided here is the use of aspect 97, wherein the nicorandil-treatable condition comprises angina (aspect 99).

Still further exemplary aspects provided here can include the following additional types of exemplary embodiments. One such further exemplary embodiment is a pharmaceutically acceptable composition comprising a therapeutically effective amount of nicorandil in a controlled release formulation dosage form that (1) when contacted with a first (low pH/acid/gastric-like) solution having a pH of 1-1.5 (a) releases a total of about 15-30% of the therapeutically effective amount after about 0.75-1.5 hours of contact with the first solution, (b) releases a total of about 20-40% of the therapeutically effective amount after about 1.5-2.5 hours of contact with the first solution, and (c) releases a total about 25-50% (e.g., about 30-50%, about 35-50%, about 30-45%, about 35-45%, or about 40-50%) of the therapeutically effective amount after about 2.5-5 hours of contact with the first solution, and (2) after contacting the composition with the first solution for a total of 2.5-5 hours and thereafter further contacting the composition with a second solution having a pH of 5.5-7 (enteric-like conditions) (a) releases a total of about 55-70% of the therapeutically effective amount (e.g., about 60-70% or 60-65% of the therapeutically effective amount) after about 1 hour of contact with the second solution, (b) releases a total of about 65-85% (e.g., about 70-80%) of the therapeutically effective amount after about 2 hours of contact with the second solution, (c) releases a total of about 80-90% (e.g., about 85%) of the therapeutically effective amount after about 6 hours of contact with the second solution, and (d) after at least about 8 hours of contact with the second solution, releases a total of at least about 90% (e.g., at least about 95%) of the therapeutically effective amount of nicorandil. In aspects, the first solution is the gastric fluid of a subject and the second is the enteric/intestinal fluid of a subject. In aspects, the first and second solutions are in vitro dissolution solutions.

In another exemplary embodiment, the invention provides a pharmaceutically acceptable composition comprising a therapeutically effective amount of one or more nicorandil compounds in a controlled release formulation dosage form that (1) releases about 15-30% of the therapeutically effective amount at about one hour after administration, (2) thereafter releases an additional detectable amount of the one or more nicorandil compounds such that about 20-40% of the therapeutically effective amount is released from the composition at about two hours after administration, (3) thereafter releases an additional detectable amount of the one or more nicorandil compounds such that about 35-50% of the therapeutically effective amount is released from the composition at about four hours after administration (4) thereafter releases an additional detectable amount of the one or more nicorandil compounds such that about 45-65% of the therapeutically effective amount is released from the composition at about six hours after administration, (5) thereafter releases an additional detectable amount of the one or more nicorandil compounds such that about 55-75% of the therapeutically effective amount is released from the composition at about seven hours after administration, (6) thereafter releases an additional detectable amount of the one or more nicorandil compounds such that about 65-85% of the therapeutically effective amount is released from the composition at about eight hours after administration, and (7) thereafter releases an additional detectable amount of the one or more nicorandil compounds such that at least about 90% of the therapeutically effective amount is released about 16 hours after administration.

In yet another exemplary aspect, the invention provides a pharmaceutically acceptable composition comprising a therapeutically effective amount of one or more nicorandil compounds in a controlled release formulation dosage form that that detectably releases the one or more nicorandil compounds from the controlled release formulation dosage form over a release period of at least about 16 hours from administration to a human patient, wherein (1) the release of the one or more nicorandil products occurs at a first release rate for a first period of about 1.5-2.5 hours, a second release rate for a second period of about 3.5-about 4.5 hours, a third release rate for a third period of about 3-about 4 hours, and a fourth release rate for a fourth period of about 6-about 7 hours, (2) about 50% or less of the one or more nicorandil compounds is released from the controlled release dosage form during the period consisting of the first period and second period, (3) the rate of release of the one or more nicorandil compounds from the controlled release dosage form, the amount of the one or more nicorandil compounds released from the controlled release dosage form, or both, during the first period is about 1.25× to 1.75× the release rate (i.e., 125% to 175% of the release rate) of the one or more nicorandil compounds during the second period or the amount released during the first period is about 1.25-1.75× the amount released during the second period, or both, (4) at least about 40% of the one or more nicorandil compounds is released from the controlled release formulation dosage form during the period consisting of the third period and the fourth period, and (5) the rate of release of the one or more nicorandil compounds during the third period being about 1.25× to about 1.75× the release rate of the one or more nicorandil compounds during the fourth period, the amount of nicorandil compound(s) released during the third period is about 1.25-1.75× the amount of nicorandil compound(s) released during the fourth period, or both.

In one embodiment, the invention provides a pharmaceutically acceptable composition comprising (1) a first release component comprising a first amount of one or more nicorandil compounds and (2) a second release component comprising a second amount of one or more nicorandil compounds and a pH-sensitive release controlling agent (an excipient that significantly releases more API at certain pH ranges, releases no significant or detectable amount of API at certain pH ranges, or both), (3) the first and second amount collectively comprising a therapeutically effective daily dose of the one or more nicorandil compounds, wherein (4) contacting the second release component and the first release component with a solution having a pH of about 1-1.5 under moderate stirring (e.g., mixing with a Turbula mixer at 50 rpm or under significantly similar conditions) for about 2 hours results in (a) about 40%-about 80% (e.g., about 50-75%, about 45-75%, or about 50-65%) of the first effective amount being released from the first release component and (b) no more than about 15% of the second effective amount being released from the second release component, and further wherein (5) contacting the second release component with a dissolution solution at a pH of 5-7, such as 5.5-7, or 6-7 at about room temperature and under moderate stirring for about 2 hours results in about 20%-about 60% (e.g., about 25-55%, about 30-50%, or about 35-45%) of the second amount being released from the second release component.

In one facet, the invention provides a pharmaceutically acceptable composition comprising (1) a therapeutically effective amount of one or more nicorandil compounds, (2) about 2-6 wt. % (e.g., about 2.5-5.5 wt. %, about 3-5 wt. %, or about 3.5-4.5 wt. %) of a pH sensitive second release polymer component (e.g., present in addition to at least one other pH sensitive polymer) that coats at least part of a core composition comprising at least some portion of the therapeutically effective amount of the one or more nicorandil compounds, (3) about 25-45 wt. % (e.g., about 27.5-42.5 wt. %, about 30-40 wt. %, or about 32-37.5 wt. %) of a controlled release polymer component that is in mixture with the therapeutically effective amount of the one or more nicorandil compounds in a core composition and that causes a portion of the therapeutically effective amount of the one or more nicorandil compounds to be released in a linear fashion for an initial period of at least about two hours from exposure to dissolution conditions, (4) about 25-60 wt. % (e.g., about 35-55 wt. %, about 40-60 wt. %, about 30-50 wt. %, or about 30-45 wt. %) of a compaction and flowability promoting agent in mixture with the one or more nicorandil compounds and the controlled release polymer component in the core composition and which has an average particle size of about 100-150 μm (e.g., about 105-135 μm, or about 105-125 μm), a bulk density of about 0.35-0.55 g/mL, and which is significantly dispersible but not significantly dissolvable in water, and (5) about 0.1% to about 0.6% (e.g., about 0.15 wt. %-about 0.45 wt. % or about 0.2 wt. %-about 0.35 wt. %) of a pharmaceutically acceptable and effective coating plasticizer.

In an aspect, the invention provides a pharmaceutically acceptable composition comprising about 3-6 pharmaceutical dosage form units, each dosage form unit (1) comprising a core composed of (a) about 7.5-12.5 wt. % (e.g., about 10 wt. %) of a nicorandil component, consisting essentially of one or more nicorandil compounds, (b) about 30-60 wt. % (e.g., about 35-55 wt. %, about 30-50 wt. %, about 35-45 wt. %, or about 35-50 wt. %) of silicified microcrystalline cellulose, (c) about 30-40 wt. % (e.g., about 35 wt. %) hydroxypropyl methylcellulose (HPMC), (d) about 0.33-1.25 wt. % magnesium stearate, and (e) optionally up to about 25% starch, and (2) about 33-66% of the pharmaceutical dosage form units being coated with a coating comprising (a) about 55-99 wt. % methacrylic acid-ethyl acrylate copolymer and (b) a triethyl citrate containing ingredient/component (e.g., about 0-30 wt. % talc, and about 0.2-1 wt. % triethyl citrate or a mixture of monoglycerides, diglycerides, and triethyl citrate (e.g., PlasAcryl® excipients (Emerson—Norristown, PA, USA or Evonik). In aspects, the (b) component is replaced with another component comprising an effective amount of a different anti-tacking agent (other than talc or mono-/di-glycerides), a different plasticizer (than triethyl citrate, such as, e.g., dibutyl sebacate, diethyl phthalate, acetylated monoglyceride, or a combination thereof), either as a substitute or in combination with the above-described anti-tacking and plasticizer agents.

In one case, the invention provides a pharmaceutically acceptable composition comprising one or more nicorandil compounds in a controlled release formulation that releases a therapeutically effective amount of the one or more nicorandil compounds in a significant number of human patients that receive a once daily administration of the composition resulting in bioavailability of nicorandil comparable to the one obtained by twice a day administration (e.g., as determined in one or more well controlled and adequate clinical trials, performed following prevailing regulatory authority standards, such as those applied in US FDA, the EMA, Health Canada, and similar regulatory agencies).

In one embodiment, the invention provides a pharmaceutically acceptable composition comprising a therapeutically effective amount of one or more nicorandil compounds in a controlled release formulation that releases the one or more nicorandil compounds at a rate such that the composition is therapeutically effective in a significant number of patients when administered in a once-daily dosing regimen (e.g., as determined in clinical trial(s)).

In one aspect, the invention provides a method of treating a nicorandil-treatable condition comprising administering a composition comprising the features of any of the above-described aspects, facets, and embodiments, to a human patient, as a once-daily treatment, wherein such a treatment regimen effectively treats the nicorandil-treatable condition in a significant number of patients in one or more well controlled and adequate studies. In one aspect, the nicorandil-treatable condition is a form of angina, such as stable angina.

The content of the original claims of this application are also incorporated herein as additional aspects of the invention.

### DETAILED DESCRIPTION OF THE INVENTION

For convenience of readers, principal features (e.g., steps/functions and elements/components) of methods, compositions, and products of the invention are described in a broad overview in this section, followed by individual descriptions of such components/elements, method steps, or related conditions/characteristics, as applicable. As indicated elsewhere herein, any aspect, facet, embodiment, or other description of aspects of the invention can be combined with any aspect, facet, embodiment, etc., contained in any other portion of this disclosure including, e.g., in the Figures and the Exemplary Aspects. Readers will particularly understand how to apply the description of the invention in the Exemplary Aspects with the disclosure provided in this Detailed Description as well as the Figures and Examples to arrive at a more complete understanding of detailed aspects of the invention.

### 1. Compositions/Formulations

As noted elsewhere terms such as “composition” and “formulation” often are used interchangeably herein to describe the pharmaceutical products including products and elements that form aspects of the invention. Compositions can be described herein as e.g., a “pharmaceutical composition,” “pharmaceutically acceptable composition,” “formulation,” “pharmaceutical formulation,” “pharmaceutically acceptable formulation” (e.g., the phrases, “formulation” and “composition” should be interpreted as interchangeable), referring to preparations comprising one or more nicorandil compounds in a form suitable for pharmaceutical administration to mammalian subjects, typically human patients, e.g., through oral administration. As noted elsewhere and understood generally, “pharmaceutical suitability” and “pharmaceutically suitable” (and, e.g., “pharmaceutically acceptable”) are phrases typically used to refer to compositions that are safe and effective for pharmaceutical administration and application, and typically means compositions/elements having sufficient potency, purity, strength, quality, and safety for pharmaceutical application, as may be judged by regulatory authority review, and as established by, e.g., one or more well controlled and adequate clinical studies performed in compliance with generally prevailing regulatory authority standards. Compositions provided here are useful in a variety of ways, including, e.g., treating symptoms or underlying causes of chronic stable angina pectoris (“CSAP”).

### A. General Characteristics of Compositions of the Invention

In aspects, compositions comprise an effective amount (e.g., a therapeutically effective amount) of one or more pharmaceutically suitable nicorandil compounds. In aspects, the nicorandil compound(s) (NCDC(s)) of the inventive compositions is/are primarily, generally, or only composed of nicorandil. In aspects, the nicorandil can be administered as a composition in a therapeutically effective amount to a human patient diagnosed with a nicorandil-treatable condition.

In aspects, compositions of the invention comprise a controlled release formulation/composition, which can include one or more controlled release elements/components. In aspects, a controlled release formulation comprises at least two different components (or component formulations—e.g., a first release formulation and a second release formulation), each having a unique composition and NCDC release profile as compared to the other (defined by, i.a., rate(s) and duration(s) of release).

E.g., in aspects a composition of the invention exhibits an extended/sustained-release profile (a profile in which, NCDC(s) are released in detectable or significant amounts for a significantly longer period than, or for a period at least about 1.25×, ≥˜1.3×, ≥˜1.35×, ≥˜1.4×, ≥˜1.45×, at least about 1.5×, ≥˜1.55×, ≥˜1.6×, ≥˜1.65×, ≥˜1.7×, ≥˜1.75×, ≥˜1.8×, ≥˜1.85×, ≥˜1.9×, ≥˜1.95×, at least about 2×, e.g., at least ˜2.5×, ≥˜2.6×, ≥˜2.7×, ≥˜2.8×, ≥˜2.9×, or at least about ˜3× as long as the period in which a detectable or significant amount of nicorandil or other NCDC is release from an immediate release (IR) formulation, such as from the currently marketed IKOREL®10 mg nicorandil IR formulation (approved for sale and available in the Netherlands, France, etc.). Typically in making such comparisons, as will be implicitly understood throughout this disclosure, a comparable amount of the same NCDC(s) are present in the compared components/compositions.

In aspects, components of a controlled release formulation exhibit delayed release (e.g., releasing at a time point significantly later than an immediate release formulation, an earlier releasing formulation, or both).

In another aspect, the controlled release composition is provided in a pharmaceutically acceptable dosage form. In an aspect, the pharmaceutically acceptable dosage form comprises at least one first release component and at least one second release component, wherein the first release component and second release are significantly different in terms of conditions of release, time of release after administration, or both. In aspects, the at least one second release component is surrounded by a coating that detectably or significantly modifies the release profile of the API from the formulation. Uncontradicted references to “API” in connection with compositions of the invention should be interpreted as mostly, generally only, or only consisting of NCDC(s), such as nicorandil.

In yet another aspect, a coating comprises an effective amount of a pH sensitive polymer that significantly limits release of the coated API/formulation/core as compared to an uncoated counterpart dosage form under certain pH conditions for at least a certain period of time (e.g., ˜2-12, ˜4-12, ˜6-12, or ˜8-12 hours). In exemplary aspects, a pH sensitive polymer significantly reduces release under gastric conditions (e.g., in solutions having a pH of about 1 to about 1.5). In aspects, the first release component and second release component are provided together within a delivery facilitating shell/vehicle, e.g., a capsule. In aspects, a delivery facilitating vehicle, such as a capsule containing ˜2-8, ˜3-6, ˜3-5, or ˜4 solid dosage forms, such as tablet dosage forms, is at least primarily composed of a release controlling polymeric material, such as HPMC.

The NCDC(s) in compositions/components (e.g., dosage forms) are combined with various functional excipients, as described further elsewhere. E.g., in an exemplary aspect, compositions comprise a controlled release polymer matrix (e.g., a matrix that comprises or mostly comprises or generally consists of/consists of HPMC), a binder/disintegrant (or two or more thereof) (e.g., a starch), and at least in part (e.g., as one sub-component or portion) a pH-responsive polymer element, such as a pH-responsive coating (e.g., a methacrylate coating or other coating that is subject to relatively higher rates of dissolution at pH 5.5 and above as compared to, e.g., pH 1.2). In aspects, such compositions are presented in multiple dosage forms (e.g., tablets, such as ˜50-150 mg tablets, e.g., about 100 mg tablets) (e.g., 3-6 thereof, such as 4 thereof, composed of 33-66% of a first release formulation dosage form(s) and 33-66% of a second release formulation dosage form(s)). In aspects, such exemplary compositions comprise dosage forms with a maximum dimension of ˜8.5 mm or less (e.g., ˜5 mm or less) in a suitable shape (e.g., a biconvex shape).

In aspects, the formulation comprises an effective amount of NCDC(s), e.g., about 10-100 mg of nicorandil, such as, e.g., ˜10 mg-˜95 mg, ˜10 mg-˜90 mg, ˜10 mg-˜85 mg, ˜10 mg-˜80 mg, ˜10 mg-˜75 mg, ˜10 mg-˜70 mg, ˜10 mg-˜65 mg, ˜10 mg-˜60 mg, ˜10 mg-˜55 mg, or ˜10 mg-˜50 mg of NCDC(s), e.g., NCDC(s) which is distributed in the various components/dosage forms of the composition.

In aspects, the formulation comprises an effective amount of NCDC(s), e.g., ˜10 mg-˜45 mg, ˜10 mg-˜40 mg, ˜10 mg-˜35 mg, ˜10 mg-˜30 mg, ˜10 mg-˜25 mg, ˜10 mg-˜20 mg, or ˜10 mg-˜15 mg of NCDC(s), e.g., NCDC(s) which is distributed in the various components/dosage forms of the composition.

In aspects, the formulation comprises an effective amount of NCDC(s), e.g., ˜15 mg-˜100 mg, ˜20 mg-˜100 mg, ˜25 mg-˜100 mg, ˜30 mg-˜100 mg, ˜35 mg-˜100 mg, ˜40 mg-˜100 mg, ˜45 mg-˜100 mg, or ˜50 mg-˜100 mg of NCDC(s), e.g., NCDC(s) which is distributed in the various components/dosage forms of the composition.

In aspects, the formulation comprises an effective amount of NCDC(s), e.g., ˜55 mg-˜100 mg, ˜60 mg-˜100 mg, ˜65 mg-˜100 mg, ˜70 mg-˜100 mg, ˜75 mg-˜100 mg, ˜80 mg-˜100 mg, ˜85 mg-˜100 mg, ˜90 mg-˜100 mg, or ˜95 mg-˜100 mg of NCDC(s), e.g., NCDC(s) which is distributed in the various components/dosage forms of the composition.

In aspects, the formulation comprises an effective amount of NCDC(s), e.g., ˜12 mg-˜90 mg, ˜14 mg-˜80 mg, ˜16 mg-˜70 mg, ˜18 mg-˜60 mg, or ˜20 mg-˜50 mg, e.g., about 15-90 mg, about 15-75 mg, about 15-60 mg, or about 20, 25, 30, 35, or 40 mg) of NCDC(s), e.g., NCDC(s) which is distributed in the various components/dosage forms of the composition. For example, in aspects, a composition can include 4 dosage forms that each contain about 5-20 mg, 5-15 mg, 5-10 mg, 7.5-20 mg, 7.5-15 mg, 7.5-10 mg, or, e.g., about 8-12 mg of NCDC(s), such as nicorandil.

Provided herewith are, e.g., figures and, e.g., tables comprising data describing various aspects of the invention. In certain aspects, the invention provides data which are within, e.g., about 75%, such as, e.g., within about 70%, within about 65%, within about 60%, within about 55%, within about 50%, within about 45%, within about 40%, within about 35%, within about 30%, within about 25%, within about 20%, within about 15%, within about 10%, within about 5%, or, e.g., within about 1%, of any one or more data points provided in tables and figures herein. Further, where specific data are not provided but where interpretation of provided exemplary data leads to the establishment of one or more exemplary characteristic(s) of the invention (e.g., among other things, where one may read a release rate from a slope of a graph provided in a figure or, e.g., among other things where one may calculate an average from a set of data, or, e.g., among other things where one may calculate a pharmacokinetic parameter from a set of data, etc.), in aspects the invention provides values which are within about 75%, such as, e.g., within about 70%, within about 65%, within about 60%, within about 55%, within about 50%, within about 45%, within about 40%, within about 35%, within about 30%, within about 25%, within about 20%, within about 15%, within about 10%, within about 5%, or, e.g., within about 1%, of such values obtained by interpretation of exemplary data provided herein.

A number of general characteristics of various exemplary compositions of the invention are provided in the Exemplary Aspects section of this disclosure. E.g., compositions having features described in any part of this section can, for example, exhibit one or more characteristics of, e.g., any one or more of aspects 1-90 or additional aspects set forth in the Exemplary Aspects of the Invention.

i. Physical Characteristics of Tablets

In aspects, composition(s) provided herein are provided in any suitable dosage form.

In certain embodiments, composition(s) are provided as tablet(s). In certain embodiments, composition(s) comprise or are provided as, at least in part, as tablet(s), wherein, e.g., formulation(s) comprise tablet component(s). In aspects the types and amounts of excipients are selected in order to achieve desired levels of size, flowability to provide for consistent filling of the tablet, hardness, and compressibility of the tablet component(s).

In aspects, tablet component(s) may have a defined hardness quality, size, and flowability meeting characteristics provided herein (e.g., ranges of such values disclosed herein). In aspects, such characteristics aid in the relevant dosage form (e.g., tablet) withstanding processing steps, e.g., compression and related processes that may occur during manufacturing.

In aspects, the tablet component(s)/dosage form(s) of a composition may have a hardness of at least about 40N (e.g., 40N-140 N, e.g., at least about 60N, such as at least about 70N, such as at least 75N, such as at least 85N, such as at least 90N (e.g., about 50-150 N, about 75-135 N, 80-140 N, about 85-135 N, or about 90-130 N), such as at about 95 N, about 100 N, about 105 N, about 110 N, about 120 N, and the like (i.e., etc.).

Various methods for the evaluation of such characteristics are known. For example, tablet hardness characteristics can be determined using crushing, fracturing, and bending test methods described in, e.g., Chang, E., Measuring Tablet Hardness: A Primer, Jun. 1, 2013, available at pharmtech.com/view/measuring-tablet-hardness-primer and according to standard tests, such as US Pharmacopeia (USP) standard 1217.

In aspects, the size of each tablet component in one or more dimensions (e.g., in its maximum dimension, in most dimensions, or in all dimensions) may be between about 3 mm and 6 mm in diameter/largest dimension, such as, e.g., ˜3.5-˜6 mm, ˜4-˜6 mm, ˜4.5-˜6 mm, ˜5-˜6 mm, ˜5.5-˜6 mm, or, e.g., ˜3 mm-˜5.5 mm, ˜3 mm-˜5 mm, ˜3 mm-˜4.5 mm, about 3 mm-˜4 mm, or, e.g., ˜3 mm-˜3.5 mm, as in, e.g., about 3.5 mm, such as about 4 mm, such as about 4.5 mm, such as about 5 mm, such as about 5.5 mm, and the like.

ii. Nicorandil Compounds (NCDCs)

In aspects, compositions comprise an effective amount of one or more NCDCs. The effective amount can be an amount disclosed herein, an amount effective in producing a physiological result disclosed herein, an amount effective in producing a therapeutic effect, such as reducing one or more symptoms or conditions associated with angina, or, e.g., combination(s) thereof. In aspects, the NCDCs comprise or mostly comprise, or generally, substantially only, or entirely consist of nicorandil. NCDCs can include any suitable pharmaceutical derivative of nicorandil, known analogs of nicorandil (e.g., US20090076095 discloses deuterated forms of nicorandil and both U.S. Pat. No. 9,359,376 and AU2020240015 disclose nicorandil derivatives), prodrugs of nicorandil (e.g., UNI-494 being developed by Unicycive Pharmaceuticals), or a pharmaceutical acceptable alternative form, such as a salt of any thereof (e.g., U.S. Pat. No. 5,211,943 purports to disclose useful salts of nicorandil). In aspects, some, most, generally all, or all of any non-nicorandil NCDCs in compositions exhibit therapeutic effects or physiological effect that are approximately the same, statistically similar to, or detectably/significantly superior to nicorandil (in comparable formulation, dosage, etc.). NCDCs are generally or entirely pharmaceutically acceptable/suitable. Non-NCD NCDCs in aspects exhibit adenosine triphosphate-sensitive potassium (KATP) channel modulation characteristics similar to or superior to nicorandil. Non-nicorandil NCDCs in aspects exhibit (at effective amounts) detectable or significant vasodilation properties. In aspects, a nicorandil compound can be a nicorandil derivative such as, e.g., 2-nicotinamidoethyl acetate, one or more nicorandil derivatives disclosed in PCT patent publication number WO2020190890, etc. In aspects, a nicorandil compound can be a nicorandil salt form, such as, e.g., any one or more nicorandil salt forms/compounds disclosed in European patent publication number EP0260452, etc. In aspects, a nicorandil compound suitable for the compositions described here are nicorandil compounds demonstrating significantly similar physiological effects as nicorandil, such as, e.g., demonstrating a similar modulation capacity of KATP channels, demonstrating similar therapeutic effect as that of nicorandil in treating a nicorandil-treatable condition, or both. In aspects, derivative(s) of nicorandil comprise 1, 2, or 3 appended atoms or groups, attached to a nicorandil core, which derivative exhibits at least significantly similar physiological or pharmaceutical/therapeutic activity as nicorandil in one or more respects, if not improvements in such respects, and remains suitable for pharmaceutical use. In aspects, each of the one or more nicorandil compounds of a composition (1) dilates peripheral and coronary resistance vessels via action on ATP-sensitive potassium channels (potassium channel activator) or (2) possesses a nitrate moiety that promotes systemic venous and coronary vasodilation.

In general, where nicorandil is described herein it implicitly simultaneously discloses a corresponding aspect comprising one or more suitable nicorandil compounds and where a reference is made to nicorandil compound(s) it implicitly describes a corresponding aspect wherein the nicorandil compound(s) is nicorandil.

In aspects, the one or more nicorandil compounds of compositions comprise, primarily comprise, generally comprise/consist of, essentially comprise/consist of, or consist only of nicorandil. In aspects, nicorandil is the only API in the composition. The structure of nicorandil is shown below:

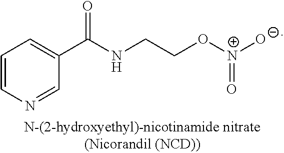 [SMILES: O=C(NCCO[N+](=O)[O-])c1cccnc1]

In aspects, a composition will comprise one or more additional APIs, such as one or more APIs that treat an aspect of a disease (including, e.g., a symptom of a disease) also treated by nicorandil, such as an aspect of angina, or that treat a related or underlying disease, condition, or symptom, such as associated pain, related physiological condition, etc.

In aspects, compositions of the invention include no APIs other than NCDCs. In aspects, compositions of the invention include no APIs other than nicorandil or the APIs of the composition generally, substantially, essentially, or entirely consist of nicorandil.

iii. Excipient Ingredients

Besides the NCDC API(s), compositions of the invention include a number of excipients that make up the formulations and, if applicable, dosage forms (and if applicable carriers/vehicle systems) of such compositions. In a general aspect, compositions can include any number of recognized excipients in the art, in suitable/effective amount, such as those characterizable as fillers, binders, bulking agents, diluents, disintegrants, glidants, coloring agents, anti-adherents, lubricants, coatings, preservatives, antioxidants, flavoring agents, sweetening agents, sorbents, solvents, co-solvents, buffering agents, chelating agents, viscosity imparting agents, surface active agents, humectants, etc. In aspects, such an excipient can be any pharmaceutically acceptable excipient that aids in the manufacturing of the tablet, aids in the performance of the tablet, or both, but does not impart significant pharmaceutical activity (e.g., is not an active pharmaceutical ingredient (API)).

Other examples of excipients that can be suitable for incorporation in pharmaceutical formulations and which can, in aspects, be included in compositions provided herein include one or more fillers (e.g., carbohydrates (e.g., glucose, lactose) calcium phosphate, calcium carbonate, cellulose, starch, polyvinyl pyrrolidone, sodium starch glycolate, etc.), one or more disintegrants (e.g., sodium carboxymethyl cellulose, gelatin, polyvinyl pyrrolidone, cellulose derivatives, polyethylene glycol, etc.), one or more binders (including solution binders such as sucrose and starch, etc. and dry binders such as cellulose, methyl cellulose, polyvinyl pyrrolidone, polyethylene glycol, etc.), one or more glidants (e.g., silica, magnesium stearate, talc, etc.), one or more lubricants (e.g., magnesium stearate, stearic acid, polyethylene glycol, sodium lauryl sulfate, paraffin, talc, etc.), one or more anti-adherents (e.g., starch, cellulose, etc.), etc., or composite ingredients (e.g., a mixture of 2, 3, or more excipients, which may exhibit 1, 2, 3, or more functions, such as a plasticizer/anti-tacking agent mixture, e.g., PlasAcryl® (sold by Evonik, Essen, Germany). In aspects, any single excipient, as evident by these few examples, can serve one or more such functions. That is, in aspects, a single excipient can serve as a binding agent or and may also improve flowability of a formulation during manufacturing, or, e.g., a single ingredient can act as a disintegrant and may also provide binding functionality.

Additional/alternative excipients can include one or more fillers in effective amount(s) (e.g., carbohydrates, e.g., sugars, such as glucose, lactose, etc., calcium phosphate, calcium carbonate, cellulose compositions, starch, polyvinyl pyrrolidone (PVP), and sodium starch glycolate, etc.); one or more disintegrants in effective amount(s) (such as gelatin, PVP, polyethylene glycol (PEG), and cellulose derivatives); binder(s) in effective amount(s) (such as cellulose, methyl cellulose, PVP, and PEG); one or more glidants in effective amount(s) (such as silicas, talc, and magnesium stearate); and one or more lubricants in effective amount(s) (such as PEG, stearic acid, magnesium stearate, sodium lauryl sulfate, paraffin, and talc).

In aspects, at least about 25%, e.g., ˜30%-˜65%, ˜35%-˜65%, ˜40%-˜65%, ˜45%-˜65%, ˜50%-˜65%, ˜55%-˜65%, or about 60%-˜65%, e.g., ˜30%-˜55%, ˜30%-˜50%, ˜30%-˜45%, ˜30%-˜40%, or ˜30%-˜35%, e.g., ˜30%-˜55%, ˜35%-˜50%, ˜40%-˜45%, or, e.g., such as about 40-55% or 40-65%, or 40-60% of a dosage form, formulation (NCDC-associated component/composition), or overall composition is composed of excipient(s) that can, i.a., be characterized as binders. Exemplary binders are described elsewhere. Additional or other alternative binders can include sugars such as sucrose, liquid glucose, sorbitol; other natural binders such as acacia, tragacanth, gelatin, starch, starch paste, pregelatinized starch, alginic acid, or cellulose; or synthetic or semisynthetic polymers such as methyl cellulose, ethyl cellulose, hydroxy propyl methyl cellulose (HPMC), hydroxy propyl cellulose, sodium carboxy methyl cellulose, polyvinyl pyrrolidine (PVP), cross-linked PVP, polyethylene glycol (PEG), polyvinyl alcohols, polymethacrylates, or microcrystalline cellulose (MCC).

In aspects, compositions comprise an effective amount of a microcrystalline cellulose (MCC). In aspects, compositions comprise a silicified microcrystalline cellulose comprising colloidal silicon dioxide and MCC. In aspects a silicified microcrystalline cellulose (SCC) component has (1) an average particle size (as determined by laser diffraction) of 65-125 um or 90-125 um or (and/or) (2) a bulk density (g/mL) of 0.25-0.37, 0.2-0.3, 0.25-0.37, 0.38-0.5, or 0.27-0.39 g/mL. An example of such an excipient is Prosolv HD90. In aspects, a composition comprises about 25-60%, about 30-55%, e.g., about 35-55% of an MCC, such as SCC. In aspects an SCC comprises about 1-3%, e.g., about 1.5-2.5%, or about 2% silicon dioxide, with the remainder of the SCC being composed of MCC. In aspects, most, generally all, or all of the MCC in a composition is composed of SCC. In aspects, a MCC or SCC makes up about 33% of any component that can be classified as a binder/disintegrant in one or more types of dosage forms or throughout an entire formulation/composition or NCDC-containing component thereof.

In aspects, a composition can optionally comprise a component that can be characterized as a binder/disintegrant or a binder/glidant, such as a starch (e.g., a corn starch or a potato starch, which can be a pregelatinized starch), in, e.g., up to about 30%, up to about 25%, or up to about 20% (e.g., about 1-30%, ˜1-25%, ˜1-20%, ˜1-15%, ˜1-10%, about 2-20%, about 5-20%, about 5-15%, about 10-20%, about 15-20% or 5-30%, 5-25%, 10-30% or 10-25%).

In aspects, as described elsewhere, a substantial component (e.g., at least about 10%, ≥˜12%, ≥˜14%, ≥˜16%, ≥˜18%, ≥˜20%, e.g., at least about 15%, etc., such as ˜10-50%, ˜10-45%, 10-40%, ˜10-35%, ˜10-30%, ˜10-25%, ˜10-20%, or, e.g., ˜15-50%, ˜20-50%, ˜25-50%, ˜30-50%, ˜35-50%, ˜40-50%, or ˜45-50%, e.g., ˜15-45%, ˜15-40%, ˜15-35%, ˜20-50%, 20-40%, 20-35%, 25-45%, 30-40%, or about 35%) of a dosage form, formulation, or product is composed of a controlled release polymer component (examples are described elsewhere). In aspects, most of the controlled release (CR) polymer (or generally all/all of the CR polymer component) is made up of hydrophilic polymers. In aspects, most, generally all, or all of the controlled release polymer component is a cellulose ether derivative polymer, such as methyl cellulose (MC), ethyl cellulose (EC), hydroxyethyl cellulose (HEC), hydroxypropyl cellulose (HPC), hydroxypropyl methylcellulose, carboxymethyl cellulose (CMC) and sodium carboxymethyl cellulose (NaCMC). In aspects, the CR polymer component is mostly, generally entirely, or entirely composed of HPMC, an example of a suitable HPMC being HPMC DC2, e.g., present at between 25-50%, such as, e.g., ˜25%-˜45%, ˜25%-˜40%, ˜25%-˜35%, ˜25%-˜30%, ˜30%-˜50%, ˜35%-˜50%, ˜40%-˜50%, ˜45%-˜50%, or ˜30%-˜45%, ˜30%-˜40%, 27.5-42%, or, e.g., about 30% or, e.g., about 35%.

In aspects, compositions comprise a lubricant, such as magnesium stearate, in about 0.25-1.5%, ˜0.5-1.5%, ˜1-1.5%, ˜0.15-1% or ˜0.25-0.5%, e.g., ˜0.75%-1.25% or about 0.33-1.25%, or another exemplified amount provided herein. In aspects, the lubricant component comprises effective amount(s) of one or more boundary (a) metallic salts of fatty acids; (b) fatty acids, hydrocarbons, and fatty alcohols; (c) fatty acid esters; (d) alkyl sulfates; (e) polymers; or (f) inorganic materials. In aspects, most, generally all, or all of the lubricant component is composed of hydrophobic materials. Exemplary lubricant materials include paraffin, talc, silica materials, fats (e.g., stearin), fatty acids (stearic acid), PEGs (e.g., PEG 400, PEG 600, etc.), sodium lauryl sulfate, sodium benzoate, and known equivalents/alternatives. In aspects, most, generally all, or all of the lubricant component is a metallic salt lubricant, such as calcium stearate. In aspects, most, generally all, or all of the lubricant component is composed of a magnesium stearate, such as, e.g., Ligamed MF-2-V (at 0.5-1%) (e.g., ˜0.5-1 mg).

In aspects compositions comprise one or more pH-responsive ingredients/components or dosage forms/formulations. In aspects, most, generally all, or all of the pH responsive component/element is composed of pH-dependent/sensitive cellulose derivatives. Examples of such polymers include cellulose acetate phthalate (CAP, cellulose acetate trimelitate (CAT), hydroxypropylmethyl cellulose phthalate (HPMCP), carboxymethylethyl cellulose (CMEC) and hydroxypropylmethyl cellulose acetate succinate (HPMCAP). Such and other pH responsive CR polymers can be mostly, generally, or entirely amorphous. In aspects, most, generally all, or all of the pH responsive polymer component is composed of a methacrylate groups (methacrylate polymer). Additional examples of pH responsive polymers and related aspects are provided elsewhere.

Additional focus will now be made on specific functional groups of excipients that, in aspects, are included in compositions, dosage forms, etc., in effective amounts.

### A. Binding Agent/Flow Agent/Disintegrant

In aspects, compositions, dosage forms, formulations, etc., can comprise one or more binding agents (“binders”). A binder can be any pharmaceutically acceptable agent capable of impacting the binding characteristics of the composition. Common binders used in tablet development include solution binders and dry binders and can include sugars such as sucrose, liquid glucose, and sorbitol, natural binders such as acacia, tragacanth, gelatin, starch, starch paste, pregelatinized starch, alginic acid, polyvinyl pyrrolidine (PVP), cross-linked PVP, polyethylene glycol (PEG), polyvinyl alcohols, polymethacrylates, cellulose-based compounds, etc. As stated above, such excipients can in aspects provide one or more functions in addition to binding functionality, such as, e.g., disintegrant or flow improvement functionality.

In aspects, one or more excipients can be characterized as a cellulose-based excipient. In aspects, a cellulose-based excipient can be any cellulose-based excipient, such as, e.g., a natural cellulose, a synthetic or semisynthetic cellulose-based polymer. In aspects, the polymer can be a hydrophilic polymer. In aspects, the polymer can be a directly compressible polymer. In aspects, a cellulose-based excipient can serve as a binding agent, as a flow agent, or both. In aspects, presence of one or more cellulose-based excipients can detectably or significantly increase the density of a tablet; detectably or significantly increase the compressibility (e.g., compaction) of a tablet, or a combination thereof over a tablet not comprising such one or more cellulose-based excipient(s). Exemplary cellulose-based excipients include cellulose, methyl cellulose, ethyl cellulose, hydroxy propyl methyl cellulose (HPMC), hydroxy propyl cellulose, sodium carboxy methyl cellulose, and microcrystalline cellulose. As noted elsewhere some of these compositions exhibit other significant/substantial functions, e.g., HPMC can be characterized as a pH-independent controlled release polymer.

In aspects, compositions comprise a mix of two or more types of binders. E.g., in aspects, a composition or a dosage form of a composition can comprise about 25-50%, e.g., ˜30-50%, ˜35-50%, ˜40-50%, or ˜45-50%, e.g., ˜25-45%, ˜25-40%, ˜25-35% or ˜25-30%, e.g., ˜30-45% or ˜25-40%, of a highly compactible binder component, and about 5-25%, e.g., ˜5-20%, ˜5-15%, ˜5-10%, e.g., ˜10-25%, ˜15-25%, or ˜20-25% of a disintegrant binder.

In aspects, the cellulose-based excipient can be a combination product comprising microcrystalline cellulose and colloidal silicon dioxide. MCC Prosolv® HD90 (also referred to specifically as SMCC Prosolv® HD90) is one suitable cellulose-based excipient suitable for use in tablets of the invention. In certain aspects, use of such a combination cellulose-based excipient product provides detectable or significant improvement of the tablet's ability to resist the degrading effects of one or more other excipients present in the tablet (e.g., magnesium stearate) over use of microcrystalline cellulose alone (e.g., microcrystalline cellulose not provided as a combination product). Examples of MCC/SCC compositions are described in, e.g., “Microcrystalline Cellulose as Pharmaceutical Excipient” by Anis Yohana Chaerunisaa, Sriwidodo and Marline Abdassah (Published: Jul. 19, 2019—DOI: 10.5772/intechopen.88092). Prosolv® excipients are available from JRS Pharma LP, Patterson, N.Y. Processes for preparing silicified microcrystalline cellulose compositions and characteristics of MCC/SCC compositions and related formulations are described in, e.g., U.S. Pat. No. 5,585,115, EP1509204A, US20100215753A1, and Aljaberi A, Chatterji A, Shah N H, Sandhu H K. Drug Dev Ind Pharm. 2009 September; 35(9):1066-71. doi: 10.1080/03639040902774131. PMID: 19353418. Functional properties associated with advantageous SCC compositions include a five-fold specific surface area increase, as well as in a 30-50% compaction increase compared to traditional MCC as well as detectable or significantly improved ingredient flow, increased compaction, content uniformity, formulation stability, or a combination of any or all thereof.

### Starch

In aspects, dosage forms/formulations of the invention can comprise one or more excipients characterizable as a starch. A starch is generally understood to mean a polysaccharide comprising a number of monosaccharides or sugar (glucose) molecules linked together. In aspects, such as starch can provide detectable or significant component/active ingredient-binding functionality to the dosage form or component thereof (e.g., tablet) and can also or alternatively serve as a diluent, filler, disintegrant, glidant, or exhibit a detectable/significant combination of any one or more of such functionalities.

In aspects, a starch can provide disintegrant functionality, aiding a tablet, for example, in breaking down into smaller fragments (dissolving) facilitating the release of API, e.g., nicorandil. In aspects, a starch can be any pharmaceutically acceptable starch, such as, e.g., a native starch such as a corn/maize or potato starch. In aspects, a starch can be a pregelatinized starch (e.g., a dried/cooked starch). In aspects, use of a modified starch detectably or significantly enhances the compressibility of the tablet over use of an unmodified starch; that is, use of a modified starch detectably or significantly reduces tablet friability. According to aspects, presence of a starch can detectably or significantly increase the water absorbing ability of the tablet over that of a tablet not comprising a starch (e.g., aiding in the suitable disintegration of the tablet(s)).

In aspects, a suitable starch for use in tablets is a starch capable of meeting compressibility requirements of the tablet, e.g., by providing a suitable size and shape of starch particles. In aspects, a starch suitable for use herein can have a size and shape profile suitable for both compression and composition flow ability. In one aspect, a suitable starch for use in tablets (or, e.g., for use in one or more other delivery form(s)) provided herein is a directly compressible starch. In aspects, STARTAB® directly compressible starch by Colorcon is a suitable starch for use in the tablets herein, having an average particle size of less than about 100 uM and a shape (e.g., generally round) advantageous for flow and compressibility.

According to aspects, compositions, formulations, or dosage forms, e.g., tablets, can comprise no starch-based excipient. In aspects, compositions, formulations, or dosage forms, e.g., tablets can comprise a starch in an amount of between about 0%-about 30%, such as, e.g., between ˜0-˜28%, ˜0-˜26%, ˜0-˜24%, ˜0-˜22%, ˜0-˜20%, ˜0-˜18%, ˜0%-˜16%, ˜0%-˜14%, ˜0%-˜12%, ˜0%-˜10%, ˜0%-˜8%, ˜0%-˜6%, ˜0%-˜4% or ˜0%-˜2%, such as, e.g., between about 2%-about 30%, ˜4%-˜30%, ˜6%-˜30%, ˜8%-˜30%, ˜10%-˜30%, ˜12%-˜30%, ˜14%-˜30%, ˜16%-˜30%, ˜18%-˜30%, ˜20%-˜30%, ˜22%-˜30%, ˜24%-˜30%, ˜26%-˜30%, ˜28%-˜30%, or, e.g., ˜2%-˜28%, ˜4%-˜26%, ˜6%-˜24%, ˜8%-˜22% or, e.g., ˜10%-˜20%. In certain aspects, compositions, formulations, or dosage forms, e.g., tablets, comprise a starch ingredient (e.g., STARTAB® directly compressible starch in an amount between 0% and about 20%.

According to aspects, compositions, formulations, or dosage forms, e.g., tablets can comprise no starch-based excipient. In aspects, tablets can comprise a starch in an amount of between about 0 mg-about 30 mg, such as, e.g., between ˜0%-˜28 mg, ˜0%-˜26 mg, ˜0%-˜24 mg, ˜0%-˜22 mg, ˜0%-˜20 mg, ˜0%-˜18 mg, ˜0 mg-˜16 mg, ˜0 mg-˜14 mg, ˜0 mg-˜12 mg, ˜0 mg-˜10 mg, ˜0 mg-˜8 mg, ˜0 mg-˜6 mg, ˜0 mg-˜4 mg or ˜0 mg-˜2 mg, such as, e.g., between about 2 mg-about 30 mg, ˜4 mg-˜30 mg, ˜6 mg-˜30 mg, ˜8 mg-˜30 mg, ˜10 mg-˜30 mg, ˜12 mg-˜30 mg, ˜14 mg-˜30 mg, ˜16 mg-˜30 mg, ˜18 mg-˜30 mg, ˜20 mg-˜30 mg, ˜22 mg-˜30 mg, ˜24 mg-˜30 mg, ˜26 mg-˜30 mg, or ˜28 mg-˜30 mg, such as, e.g., ˜2 mg-˜28 mg, ˜4 mg-˜26 mg, ˜6 mg-˜24 mg, ˜8 mg-˜22 mg, or ˜10 mg-˜20 mg. In certain aspects, tablets comprise a starch ingredient (e.g., STARTAB® directly compressible starch in an amount between 0-about 20 mg.

b. Controlled Release Polymers (Generally)

According to aspects, composition(s) provided herein comprise a controlled release gastric acid dissolution susceptible component (GADSC). In aspects, composition(s) provided herein comprise a controlled release gastric acid dissolution resistant component (GADRC). In aspects, composition(s) provided herein comprise both a GADSC and a GADRC. In aspects, a GADSC, a GADRC, or both a GADSC and a GADRC comprise one or more release retardant material(s).

Numerous types of release retardant materials are provided herein, and equivalents thereof known in the art. To exemplify such an embodiment, significant disclosure is provided herein regarding compositions comprising ion exchange resin components as a part of a release retardant component/element alone or with other release retardant agents/elements (e.g., one or more release retardant coating polymers). While such formulations are a unique aspect of the invention, such detailed disclosure is not intended to detract those of ordinary skill in the art from employing other release retardant agents to achieve compositions having the characteristics described herein, which the inventors conceive are suitable as therapeutic agents according to the various aspects of the invention provided throughout this disclosure.

In general, the release retarding agent comprises a pharmaceutically acceptable polymer, hydrophilic or hydrophobic polymer such as ethylcellulose, methylcellulose, hydroxypropylmethylcellulose (HPMC), hydroxyethylcellulose (HEC), acrylic acid ester, e.g. Eudragit RL 100, Eudragit RLPO, Eudragit RL 30 D, Eudragit RSPO, Eudragit RS 30 D, Eudragit NE 30 D, Eudragit NE 40 D, cellulose acetate phthalate, HEC phthalate, HPMC phthalate or other cellulosic polymers, or mixtures of polymers.

Release retardant agents can comprise or be associated with a water-insoluble polymer, a pH-dependent polymer, or both (e.g., derivatives such as cellulose ethers, cellulose esters, polymethacrylic acid ester copolymers, aminoalkyl methacrylate copolymers, a copolymer of polyvinyl acetate, polyvinylpyrrolidone etc., methyl acrylate acrylic acid copolymer, methyl acrylate methacrylic acid copolymer, butyl acrylate styrene acrylic acid copolymer, etc. Release controlling agent(s) can include known release-controlling polymers such as ethylcellulose, polyvinyl acetate, polyacrylate and polymethacrylate, copolymers thereof which provide extended release. In aspects, extended-release coatings comprising a water-insoluble polymer and a pH-dependent polymer can be used in compositions. In aspects, controlled release coating for a composition can comprise ethyl cellulose and polyethylene glycol. In aspects, a release-retarding agent is selected from the group consisting of hydroxypropyl cellulose (HPC), hydroxypropyl methyl cellulose (HPMC or hypromellose), methylcellulose (MC), hydroxyethyl cellulose (HEC), polyvinyl pyrrolidone (PVP) and xanthan gum, etc. In aspects, a release controlling polymer/coating comprise at least one hydrophobic release controlling agent and at least one hydrophilic release controlling agent. In aspects, release controlling materials comprise hydroxypropyl methylcellulose acetate succinate (HPMCAS), HPMC phthalate, polyvinyl acetate phthalate, or methacrylic acid copolymers, etc. In aspects, controlled release agents/release retardant agents comprise hydroxypropyl methylcellulose (HPMC) acetate succinate, HPMC phthalate, polyvinyl acetate phthalate, methacrylic acid copolymers, etc.

In aspects, a release-retarding agent (CR polymer—such as a pH independent CR polymer) is selected from the group consisting of hydroxypropyl cellulose (HPC), hydroxypropyl methyl cellulose (HPMC or hypromellose), methylcellulose (MC), hydroxyethyl cellulose (HEC), polyvinyl pyrrolidone (PVP) and xanthan gum, etc. In aspects, a release controlling polymer/coating comprise at least one hydrophobic release controlling agent and at least one hydrophilic release controlling agent. In aspects, release controlling materials comprise hydroxypropyl methylcellulose acetate succinate (HPMCAS), HPMC phthalate, polyvinyl acetate phthalate, or methacrylic acid copolymers, etc. In aspects, controlled release agents/release retardant agents comprise hydroxypropyl methylcellulose (HPMC) acetate succinate, HPMC phthalate, polyvinyl acetate phthalate, methacrylic acid copolymers, etc.

A retarding agent in the form of a rate-controlling polymer can be, in aspects, selected from hydrophilic polymer, hydrophobic polymer or the mixture thereof. A rate controlling polymer can be used as a matrixing agent, coating agent, or both. Hydrophilic polymers can include hydroxyethyl cellulose, hydroxypropyl cellulose, sodium alginate, carbomer, sodium carboxymethyl cellulose, xanthan gum, guar gum, locust bean gum, polyvinyl alcohol, and hydroxypropyl methylcellulose. Hydrophobic polymers that can be used can comprise cellulose ether compositions, e.g., ethyl cellulose, cellulose acetate, polyvinyl acetate, methacrylic acid esters neutral polymer, polyvinyl alcohol-maleic anhydride copolymers, HPMC phthalate, EUDRAGIT® RSPO, EUDRAGIT® S 100, hydrogenated castor oil, waxes, etc.

In aspects, the release resistant polymer of the first release component is significantly less pH responsive than the second release component, does not exhibit significant pH sensitivity/responsiveness or at least is not resistant to gastric acid dissolution conditions. In aspects, the release retardant nature/characterization of such formulations and components thereof (e.g., polymers contained there, like HPMC) is a reflection of the controlled release of the formulation as compared to IR dosage forms, particularly at conditions other than gastric acid dissolution conditions.

In aspects, the cellulose-based excipient can comprise a cellulose ether derivative, such as, e.g., a methyl cellulose, ethyl cellulose, hydroxyethyl cellulose, hydroxypropyl cellulose, hydroxypropyl methylcellulose (HPMC), carboxymethyl cellulose, sodium carboxymethyl cellulose, etc. In aspects, tablets comprise HPMC. In aspects, presence of HPMC detectably or significantly improves upon the controlled release of the API (e.g., nicorandil) as the tablet dissolves over that of a tablet not comprising HPMC. In certain aspects, a suitable HPMC which can be present in tablets provided by the invention is a Methocel™ DC2 polymer. In aspects, such Methocel™ DC2 polymer provides a detectable or significantly better performance of the HPMC over other HPMC products due to the spherical morphology of DC2 polymer particles.

In aspects, compositions, formulations, or dosage forms, e.g., tablets, comprise both a microcrystalline cellulose-based excipient and a cellulose ether derivative such as HPMC. In aspects, compositions herein comprise a microcrystalline cellulose-based excipient present as a combination product, such as MCC Prosolv®HD90 (microcrystalline cellulose and colloidal silicon dioxide). In aspects, tablets comprise both Prosolv®HD90 and Methocel™ DC2 polymer (HPMC).

In aspects, tablets can comprise any single cellulose-based excipient ingredient in an amount between about 20%-about 60%, e.g., ˜20%-˜55%, ˜20%-˜50%, ˜20%-˜45%, ˜20%-˜40%, ˜20%-˜35%, ˜20%-˜30%, ˜20%-˜25%, or, e.g., ˜25%-˜60%, ˜30%-˜60%, ˜35%-˜60%, ˜40%-˜60%, ˜45%-˜60%, ˜50%-˜60%, or ˜55%-˜60%, such as, e.g., between ˜22%-˜58%, ˜24%-˜56%, or, e.g., ˜26%-˜55%, such as for example between about 26%-˜54%, ˜26%-˜52%, ˜26%-˜50%, ˜26%-˜48%, ˜26%-˜46%, ˜26%-˜44%, or, e.g., between ˜26%-˜42%, such as for example between about 28%-about 56%, ˜30%-˜56%, ˜32%-˜56%, or, e.g., ˜34%-˜56% of any single cellulose-based excipient. In a specific aspect, tablets can comprise between about 34%-about 54.5% of microcrystalline cellulose (MCC) Prosolv®HD90. In another specific aspect, tablets can comprise between about 27.5%-about 42% HPMC (e.g., HPMC DC2 (Methocel™ HPMC DC2 polymer). In aspects, HPMC can be present in an amount wherein the ratio of one or more nicorandil compounds to HPMC is in an amount between about 1:2 and 1:5, such as 1:2.5, 1:3, 1:3.25, 1:3.5, 1:3.75, 1:4, 1:4.25, 1:4.5, 1:4.75, etc.

In aspects, compositions, formulations, or dosage forms, e.g., tablets can comprise any single cellulose-based excipient ingredient in an amount between about 20 mg-about 60 mg, such as, e.g., ˜20 mg-˜55 mg, ˜20 mg-˜50 mg, ˜20 mg-˜45 mg, ˜20 mg-˜40 mg, ˜20 mg-˜35 mg, ˜20 mg-˜30 mg, or ˜20 mg-˜55 mg, e.g., ˜25 mg-˜60 mg, ˜30 mg-˜60 mg, ˜35 mg-˜60 mg, ˜40 mg-˜60 mg, ˜45 mg-˜60 mg, ˜50 mg-˜60 mg, or ˜55 mg-˜60 mg, such as between ˜22 mg-˜58 mg, ˜24 mg-˜56 mg, or, e.g., ˜26 mg-˜55 mg, such as for example between about 26 mg-˜54 mg, ˜26 mg-˜52 mg, ˜26 mg-˜50 mg, ˜26 mg-˜48 mg, ˜26 mg-˜46 mg, ˜26 mg-˜44 mg, or, e.g., between ˜26 mg-˜42 mg, such as for example between about 28 mg-about 56 mg, ˜30 mg-˜56 mg, ˜32 mg-˜56 mg, or, e.g., ˜34 mg-˜56 mg of any single cellulose-based excipient. In a specific aspect, tablets can comprise between about 34-about 54.5% of microcrystalline cellulose (MCC) Prosolv®HD90. In another specific aspect, tablets can comprise between about 27.5%-about 42% HPMC DC2 (Methocel™ DC2 HPMC polymer).

In aspects, compositions, formulations, or dosage forms, e.g., tablets can comprise between about 55%-about 97.5%, such as 55%-95%, or 55%-90% in total of a combination of two or more cellulose-based ingredient(s), such as, e.g., between ˜55%-˜95%, ˜60%-˜95%, ˜65%-˜95%, ˜70%-˜95%, ˜75%-˜95%, ˜80%-˜95%, ˜85%-˜95%, ˜90%-˜95%, or ˜60%-˜97.5%, ˜65%-˜97.5%, ˜70%-˜97.5%, ˜75%-˜97.5%, ˜80%-˜97.5%, ˜85%-˜97.5%, ˜90%-˜97.5%, or ˜95%-˜97.5%, e.g., ˜56%-˜90%, ˜57%-˜90%, ˜58%-˜90%, ˜59%-˜90%, ˜60%-˜90%, or, e.g., between about 61%-˜90% of two or more cellulose-based ingredients. In aspects, such two or more cellulose based ingredients can be a microcrystalline cellulose, such as Prosolv®HD90 (microcrystalline cellulose and colloidal silicon dioxide) and HPMC (e.g., HPMC DC2 (Methocel™ DC2 HPMC polymer). In aspects the cellulose-based ingredients detectably or significantly increase compactability/compaction of the formulation. In aspects the cellulose-based ingredients detectably or significantly increase flowability of the formulations. It yet another aspect the cellulose-based ingredients can act as binders.

In aspects, HPMC can be present in an amount in a dosage form, formulation, etc., wherein the ratio of one or more nicorandil compounds to HPMC is in an amount between about 1:2 and 1:5, such as 1:2.5, 1:3, 1:3.25, 1:3.5, 1:3.75, 1:4, 1:4.25, 1:4.5, 1:4.75, etc.

c. Plasticizers

In aspects, compositions, formulations, or dosage forms comprise an effective amount of a plasticizer component. In aspects, a plasticizer component can be composed of any suitable pharmaceutically acceptable plasticizer, such as dibutylsebacate, vegetable oil, diethylsebacate, diethylphthalate, tricetin or propylene glycol. A plasticizer is typically an element of a coating, such as a pH responsive polymer-based coating that makes up a gastric acid dissolution resistant component (GADRC) of a composition of the invention. In aspects, some, most, generally all, or all of the plasticizer component is composed of triethylcitrate (TEC). In aspects, a composition or dosage form coating comprises about 0.25-1.25%, ˜0.25-1%, 0.25-0.75%, 0.25-0.5%, 0.5-1.25%, 0.75-1.25%, or 1-1.25%, such as 0.33-1% or 0.5-1%, or 0.5-0.75% of a plasticizer component. In aspects, the plasticizer component comprises an effective amount of one or more of diethyl phthalate, glyceryl triacetate, glyceryl monocaprylate and triethyl citrate.

In aspects, hydrophilic cellulose ether derivatives, e.g., HPMC, also or alternatively can be used with enteric polymer for improving the film forming and plasticity of the main enteric polymer (along with or apart from the above-described plasticizer(s)). In aspects, HPMC is used in enteric coating process as pre-coating or sub-coating polymer due to its very good film forming properties and suitable polymer-to-polymer adhesion with enteric coating polymers especially with cellulose ester derivatives such as CAP, HPMCP, HPMCAS, CMEC, and CAT.

In aspects, a coating of a dosage form of compositions of the invention comprises a coating comprising an effective amount of a plasticizer, such as a TEC plasticizer. In aspects, the TEC plasticizer is derived from an ingredient comprising TEC and other components/ingredients. E.g., in aspects the plasticizer comprises, mostly comprises, generally consists of, or consists of a composition having approximately the same composition as a PlasACRYL® (e.g., PlasACRYL® or PlasACRYL® T20). In aspects, most, generally all or all of the plasticizer component of a dosage form/composition contains an effective mixture of an anti-tacking agent, a plasticizer (e.g., TEC), and a stabilizer and, in aspects, optionally exhibits significantly similar functionality to a PlasACRYL® ingredient.

d. Lubricant Excipients

In aspects, compositions, formulations, or dosage forms, e.g., tablets can comprise any one or more pharmaceutically acceptable excipients characterizable as a lubricant. In aspects, a lubricant can be any pharmaceutically acceptable lubricant such as, e.g., a hydrophobic lubricant. In aspects, a lubricant can be soluble. In aspects, a lubricant can be insoluble. In aspects, such lubricant excipient(s) can be boundary lubricant(s). In aspects, such boundary lubricant(s) can be, e.g., metallic salts of fatty acids; fatty acids, hydrocarbons, and fatty alcohols; fatty acid esters; alkyl sulfates; polymers; or, e.g., inorganic materials. In aspects, an excipient characterizable as a lubricant can detectably or significantly prevent adherence of a composition (e.g., a powder prior to tableting) to punch die/faces and thus can detectably or significantly reduce sticking upon attempts to eject tablets from a die after compaction. Exemplary lubricants common in the art include, e.g., paraffin, minerals such as talc or silica, fats (e.g., vegetable stearin, magnesium stearate, calcium stearate, or stearic acid), sodium lauryl sulphate, sodium benzoate, polyethylene glycols (PEGs) (e.g., PEG 400, PEG 600, etc.), etc. In aspects, such an excipient can also be referred to as an anti-adherent.

In aspects, a lubricant is a material having a high specific surface area and, e.g., a fine particle size. In aspects, such a suitable or optimal specific surface area can be, e.g., between about 4-12 m2/g, such as, e.g., between about 5-11 m2/g, or between about 6-10 m2/g. In aspects, a suitable or optimal median particle size (D50) can be between about 4-about 14 μm, such as, e.g., between about 5-13 μm, about 6-12 μm, or, e.g., between about 7-11 μm. In aspects, lubricants having such characteristics detectably or significantly increase the releasing speed of a tablet during tablet pressing over that of lubricants having characteristics outside of such exemplary boundaries, though this does not mean that lubricants outside of these boundaries would not also be suitable for use in the tablets herein. In aspects, lubricants having such characteristics as those described in this paragraph can aid in the provision of consistent physical performance of tablets, e.g., such as consistent hardness and dissolution profiles.

In aspects, dosage forms (e.g., tablets) of compositions can comprise one or more salts. In aspects, salts can be any pharmaceutically acceptable salt serving one or more excipient functions as described above. In aspects, the tablet can comprise one or more salts characterizable as a lubricant. In aspects, such an excipient can be a salt of a fatty acid, such as, e.g., a salt of stearic acid. In aspects, such a salt can be a magnesium salt or a calcium salt, such as, e.g., a magnesium salt of stearic acid (magnesium stearate) or a calcium salt of stearic acid (calcium stearate). In aspects, other lubricants can include, e.g., hydrous magnesium silicate or talc. In aspects, tablets herein comprise magnesium stearate. In certain aspects, tablets herein comprise magnesium stearate provided as LIGAMED® MF-2-V.

In aspects, presence of an effective amount of magnesium stearate in a tablet/other dosage form detectably or significantly reduces manufacturing down time, increases tablet throughput (e.g., increases tablet production volume), reduces manufacturing costs, or a combination thereof by detectably or significantly improving the clean ejection of tablets from compacting equipment post compaction; that is, detectably or significantly reducing sticking of the compacted tablet or composition material to compacting equipment.

In aspects, tablets/dosage forms can comprise one or more excipients characterizable as a lubricant in an amount of between about 0.1%-about 3%, e.g., ˜0.1%-˜0.1%-˜2.5%, e.g., ˜0.1%-˜2%, ˜0.1%-˜1%, or ˜0.1%-˜0.5%, or, e.g., ˜0.5%-˜3%, ˜1%-˜3%, ˜1.5%-˜3%, ˜2%-˜3%, or, e.g., ˜2.5%-˜3%, such as, e.g., between ˜0.2%-2.5%, ˜0.3%-˜2.0%, ˜0.4%-˜1.5%, or, e.g., ˜0.5%-˜1%, such as, e.g., ˜0.6%-1%, ˜0.7%-˜1%, ˜0.8-˜1%, or ˜0.9-˜1%, such as, e.g., between about 0.5%-about 0.9%, ˜0.5%-˜0.8%, ˜0.5%-˜0.7%, or, e.g., ˜0.5%-˜0.6% of a lubricant, such as, e.g., magnesium stearate, such as, e.g., LIGAMED® MF-2-V. Readers will understand that dosage forms often are tablets in compositions of the invention and, accordingly, disclosure of aspects involving tablets, uncontradicted, provide support for suitable solid dosage forms, generally.

In aspects, tablets can comprise one or more excipients characterizable as a lubricant in an about of between about 0.1 mg-about 3 mg, such as, e.g., ˜0.1 mg-˜2.5 mg, ˜0.1 mg-˜2 mg, ˜0.1 mg-˜1.5 mg, ˜0.1 mg-˜1 mg, or ˜0.5 mg-˜3 mg, ˜1 mg-˜3 mg, ˜1.5 mg-˜3 mg, ˜2 mg-˜3 mg, or ˜2.5 mg-˜3 mg, such as between ˜0.2 mg-2.5 mg, ˜0.3 mg-˜2.0 mg, ˜0.4 mg-˜1.5 mg, or, e.g., ˜0.5 mg-˜1 mg, such as, e.g., ˜0.6 mg-1 mg, ˜0.7 mg-˜1 mg, ˜0.8 mg-˜1 mg, or ˜0.9 mg-˜1 mg, such as, e.g., between about 0.5 mg-about 0.9 mg, ˜0.5 mg-˜0.8 mg, ˜0.5 mg-˜0.7 mg, or, e.g., ˜0.5 mg-˜0.6 mg of a lubricant, e.g., magnesium stearate, such as, e.g., LIGAMED® MF-2-V.

Compression lubricants prevent adherence of granule/powder to punch die/faces and promote smooth ejection from the die after compaction. Lubricants can also be used when compression isn't involved such as in powder blends for filling into capsules to prevent adherence of granule/powder to equipment surfaces and dosator mechanisms and coating the surface of multi-particulate dosage forms to inhibit agglomeration of individual particles. Minerals such as talc or silica, and fats, e.g. vegetable stearin, magnesium stearate or stearic acid are the most frequently used lubricants in tablets or hard gelatin capsules. The most widely used lubricants in use today are hydrophobic lubricants. These are usually effective at relatively low concentrations and many also have both anti-adherent and glidant properties.

Relevant principles, compositions, and methods relating to lubricant components are known in the art (see, e.g., Li and Wu, Lubricants in Pharmaceutical Solid Dosage Forms, Lubricants 2014, 2, 21-43; doi:10.3390/lubricants2010021, ISSN 2075-4442, mdpi.com/journal/lubricants).

iv. Ratios

According to aspects, any component(s) or compound(s)/agent(s)/constituent(s) described herein can be present in composition(s)/formulation(s) in therapeutically effective amount(s), compositionally compatible amount(s), or both. In aspects, any single component or compound/agent provided herein can be present in a relationship with, such as, e.g., in a ratio with, any one or more other single component or compound/agent. In aspects, any combination of component(s) or compound(s)/agent(s) provided herein can be present in a ratio with any other combination of component(s) or compound(s)/agent(s). In aspects, ratio(s) between such component(s) or compound(s)/agent(s) or combinations thereof can be established using any provided amounts for each disclosed herein, including, e.g., values within ranges of such amounts disclosed herein. Certain exemplary ratios are provided in embodiments throughout this disclosure.

### B. First Release Component

According to aspects, upon maintaining the composition in contact with a dissolution media having a pH of between about 1 and about 1.5 (e.g., a pH of ˜1, ˜1.1, ˜1.2, ˜1.3, ˜1.4, or a pH of about 1.5) for a period of about 4 hours result in the composition releasing no more than about 50%, e.g. ≤˜48%, ≤˜46%, ≤˜44%, ≤˜42%, ≤˜40%, ≤˜38%, ≤˜36%, ≤˜34%, ≤˜32%, ≤˜30%, ≤˜28%, ≤˜26%, ≤˜24%, ≤˜22%, or ≤˜20%, of the NCDC(s), e.g., nicorandil, contained in the composition.

In aspects, compositions comprise at least a first release component and a second release component. In aspects, the first and second release components have detectably or significantly different NCDC release characteristics (e.g., significantly different release under one or more pH conditions). In aspects, compositions comprise one or more first release dosage form(s), which typically are solid first dosage form(s), which can be, for examples, tablets. In aspects, the one or more first release tablet component(s) comprises a first part (or, i.e., a first portion) of the therapeutically effective amount of NCDC(s), e.g., nicorandil, present in the composition. In aspects, the one or more first release tablet component(s) (“first release tablet(s)”) comprise(s) nicorandil or a therapeutically equivalent one or more NCDC(s) in an amount of between about 5 mg and 20 mg, e.g., ˜5 mg-˜18 mg, ˜5 mg-˜16 mg, ˜5 mg-˜14 mg, ˜5 mg-˜12 mg, ˜5 mg-˜10 mg, or ˜5 mg-˜8 mg, e.g., ˜6 mg-˜20 mg, ˜8 mg-˜20 mg, ˜10 mg-˜20 mg, ˜12 mg-˜20 mg, ˜14 mg-˜20 mg, ˜16 mg-˜20 mg, or ˜18 mg-˜20 mg, such as, e.g., ˜6 mg-˜18 mg, ˜8 mg-˜16 mg, ˜10 mg-˜14 mg, or, e.g., between about 7.5 mg and 17.5 mg, such as between about 9 mg and 15 mg, such as between about 9.5 mg and 12 mg. In aspects, each first release tablet comprises about 10 mg of NCDC(s), e.g., nicorandil.

In aspects, the first release component comprises about 33-66% (e.g., ˜33%-60%, ˜33%-55%, ˜33%-50%, ˜33%-45%, or ˜33%-40%, e.g., ˜35%-˜66%, ˜40%-66%, ˜45%-˜66%, ˜50%-˜66%, ˜55%-˜66%, or ˜60%-˜66%) of the NCDC(s) in the composition, makes up about 33-66% (e.g., ˜33%-60%, ˜33%-55%, ˜33%-50%, ˜33%-45%, or ˜33%-40%, e.g., ˜35%-˜66%, ˜40%-66%, ˜45%-˜66%, ˜50%-˜66%, ˜55%-˜66%, or ˜60%-˜66%) of the composition, or both.

In aspects, the first release component, overall, comprises about 10-60 mg of NCDC(s), e.g., ˜15-60 mg, ˜20-60 mg, ˜25-60 mg, ˜30-60 mg, ˜35-60 mg, ˜40-60 mg, ˜45-60 mg, ˜50-60 mg, or ˜15-60 mg, e.g., ˜10-55 mg, ˜10-50 mg, ˜10-45 mg, ˜10-40 mg, ˜10-35 mg, ˜10-30 mg, ˜10-25 mg, ˜10-20 mg, or ˜10-15 mg, such as about 15-55 mg, about 15-50 mg, about 15-45 mg, about 15-40 mg, about 15-30 mg, about 10-50 mg, about 10-45 mg, about 10-40 mg, about 20-40 mg, about 20-35 mg, about 10-30 mg, about 10-25 mg, or about 10-20 mg of one or more NCDCs.

In aspects, the first release component is not characterizable as a pH responsive component. In aspects, the first release component is characterizable as a general (pH independent) controlled release formulation. In aspects, the first release dosage form(s) is characterizable as a gastric acid dissolution susceptible component (GADSC). In aspects, a gastric acid dissolution susceptible component comprises a release resistant retardant polymer component.

In aspects, contacting the first release component (e.g., one or more first release tablets) with a dissolution medium having a pH of between about 1 and 1.5 (which mimics the acidic environment of the stomach), such as about 1.1, such as about 1.2, such as about 1.3, or such as a pH of about 1.4, at a suitable temperature, e.g., at a temperature approximating normal human body temperature, e.g., about 37° C., and, further optionally, under moderate stirring (as exemplified below) for about 2 hours results in the release of the one or more nicorandil compounds in the first release formulation such that, e.g., the amount of the one or more nicorandil compounds released from the first release component/GADSC is mostly, generally, or entirely in accordance with one or more elements of the following release profile characteristics: (1) about 0.5-25%, e.g., about 5-20%, about 5-15%, or about 12% of the nicorandil compound(s) being released from the GADSC after 0.25 hours in the dissolution media; (2) about 11.5% to about 44.5%, such as about 20% to about 35%, such as about 28% of the nicorandil compound(s) (NCDC(s)) released after 1 hour in the dissolution media; (3) about 25% to about 60%, such as about 32% to about 52%, such as about 42.5% of the NCDC(s) released at 2 hours in the dissolution media; (4) about 60-92.5%, e.g., about 65-85%, such as about 75% or about 76% of the NCDC(s) released at 6 hours in the dissolution media; (5) about 71% to about 98%, such as about 80% to about 90%, such as about 84.5% of the NCDC(s) released at 8 hours of contact with the dissolution media, and (6) about 83% to about 100%, such as about 87% to about 97%, e.g., about 92.25% of the NCDC(s) in the GADSC being released at 14 hours of contact with the dissolution media.

According to aspects, contacting the first release component (e.g., one or more first release tablets) with a dissolution medium having a pH of between about 1 and 1.5 (e.g., ˜1-1.4, ˜1-1.3, ˜1-1.2, ˜1-1.1, ˜1.1-1.4, ˜1.2-1.4, or ˜1.3-˜1.4, e.g., ˜1.2-1.4) at a suitable temperature, and, further optionally, under moderate stirring (as exemplified below), results in about 0.5-25%, ˜1-25%, ˜5-25%, ˜10-25%, ˜15-25%, ˜2-25%, or ˜0.5-20%, ˜0.5-15%, ˜0.5-10%, or ˜0.5-5%, e.g., about 5-20%, about 5-15%, about 10-15%, or about 12% of the nicorandil compound(s) being released from the GADSC after 0.25 hours in the dissolution media.

According to aspects, contacting the first release component (e.g., one or more first release tablets) with a dissolution medium having a pH of between about 1 and 1.5 at a suitable temperature, and, further optionally, under moderate stirring (as exemplified below), results in about 11.5% to about 44.5%, e.g., ˜11.5%-˜40%, ˜11.5%-˜35%, ˜11.5%-˜30%, ˜11.5%-˜25%, ˜11.5%-˜20%, ˜11.5%-˜15%, e.g., ˜15%-˜44.5%, ˜20%-˜44.5%, ˜25%-˜44.5%, ˜30%-˜44.5%, ˜35%-˜44.5%, or ˜40%-˜44.5%, such as, e.g., about 15% to about 40%, about 15% to about 30%, about 20% to about 35%, or such as about 28% of the nicorandil compound(s) (NCDC(s)) released after 1 hour in the dissolution media.

According to aspects, contacting the first release component (e.g., one or more first release tablets) with a dissolution medium having a pH of between about 1 and 1.5 at a suitable temperature, and, further optionally, under moderate stirring (as exemplified below), results in about 25% to about 70%, e.g., ˜25%-˜65%, ˜25%-˜60%, ˜25%-˜55%, ˜25%-˜50%, ˜25%-˜45%, ˜25%-˜40%, ˜25%-˜35%, ˜25%-˜30%, e.g., ˜30%-˜70%, ˜35%-˜70%, ˜40%-˜70%, ˜45%-˜70%, ˜50%-˜70%, ˜55%-˜70%, ˜60%-˜70%, or ˜65%-˜70%, such as, e.g., about 30% to about 65%, about 35% to about 55%, about 35% to about 45%, about 40%-about 45%, about 32% to about 52%, or, e.g., about 42.5% of the NCDC(s) released at 2 hours in the dissolution media.

According to aspects, contacting the first release component (e.g., one or more first release tablets) with a dissolution medium having a pH of between about 1 and 1.5 at a suitable temperature, and, further optionally, under moderate stirring (as exemplified below), results in about 60-92.5%, such as, e.g., ˜80-100%, for example ˜65-92.5%, ˜70-92.5%, ˜75-92.5%, ˜80-92.5%, ˜85-92.5%, or ˜90-92.5%, e.g., ˜60-90%, ˜60-85%, ˜60-80%, ˜60-75%, ˜60-70%, ˜60-65%, e.g., about 65-90%, about 65-85%, or about 70-80%, such as ˜85%-˜100%, ˜90%-˜100%, or ˜95%-˜100%, ˜80%-˜95%, ˜80%-˜90%, or ˜80%-˜85%, e.g., about 75% or about 76% of the NCDC(s) released at 6 hours in the dissolution media.

According to aspects, contacting the first release component (e.g., one or more first release tablets) with a dissolution medium having a pH of between about 1 and 1.5 at a suitable temperature, and, further optionally, under moderate stirring (as exemplified below), results in about 71% to about 98%, such as, e.g., ˜71%-˜95%, ˜71%-˜90%, ˜71%-˜85%, ˜71%-˜80%, or ˜71%-˜75%, e.g., ˜75%-˜98%, ˜80%-˜98%, ˜85%-˜98%, ˜90%-˜98%, or ˜95%-˜98%, such as for example ˜75%-˜95%, ˜80%-˜90%, such as about 80% to about 90%, such as about 84.5% of the NCDC(s) released at 8 hours of contact with the dissolution media.

According to aspects, contacting the first release component (e.g., one or more first release tablets) with a dissolution medium having a pH of between about 1 and 1.5 at a suitable temperature, and, further optionally, under moderate stirring (as exemplified below), results in about 83% to about 100%, e.g., ˜85%-˜100%, ˜90%-˜100%, ˜95%-˜100%, e.g., ˜83%-˜95%, ˜83%-˜90%, ˜83%-˜85%, such as about 87% to about 97%, e.g., ˜92.25% of the NCDC(s) in the GADSC being released at 14 hours of contact with the dissolution media.

In aspects, under the same or approximately similar dissolution conditions, about 90% of the nicorandil in the first release tablets is released, within between about 4 to about 7 hours, such as, e.g., within between about 5 hours and ˜6 hours after administration to a subject, between about 5-6 hours from contact with a first solution having a pH of about 1-1.5, or any or all thereof. Uncontradicted, terms such as “approximately similar” mean “about the same” (having mostly, generally, or only conditions/characteristics that are about equivalent, such as within +/−10% of any indicated amounts, measures, etc. Uncontradicted description of a time that is “about” another time of less than 18 hours means within +/−15 minutes of the referenced time and provides support for within about 10 minutes or 5 minutes of the referenced time.

In aspects, compositions of the invention comprise 1-8, 1-6, 1-5, 1-4, 2-8, 3-8, 3-7, 3-6, 3-5, or 4 solid dosage forms (which also can be called “units” or “dosage form units”). In aspects, the solid dosage forms/units can be characterized as tablets.

In general, any dosage form herein described as a tablet implicitly discloses a corresponding aspect comprising a solid dosage form, in general, and vice versa. Other solid dosage forms/units for oral administration include capsules, sachets, films, and the like.

In aspects, a solid dosage form has a limited surface area, such as that obtained through a tablet formulation (i.e., is significantly similar in surface area to a tablet formulation dosage form, such as any of those specifically described herein). The solid dosage forms can have any suitable shape and size. In aspects, each solid dosage form is about 3-6 mm (0.3-0.6 cm) in diameter or in its largest dimension (or in all dimensions). In aspects, each solid dosage form has a height/thickness of about 0.2-0.6 cm, e.g., ˜0.3-0.5 cm, or about 0.3-0.4 cm, such as about 0.25-0.35 cm, about 0.3 cm, or about 0.35-0.4 cm. In aspects, some, most, generally all, or all of the solid dosage forms of a composition are circular in shape or disc shaped.

In aspects, about 25%, about 33%, about 40%, about 50%, about 60%, about 66%, or about 75% of the solid dosage forms of a composition are first release dosage forms/tablets. In aspects, a composition comprises about 1-6, 2-6, 1-5, 2-5, 1-4, 1-3, 2-4, 2-3, or 2 first release dosage forms/tablets, each of the first release dosage forms comprising a first release formulation (e.g., a GADS/pH independent controlled release formulation). In aspects, the composition further comprises at least one second release dosage form, which, as described below, has a different, and typically significantly delayed/modified, release profile, as compared to the first release profile associated with the first release dosage forms/units.

In aspects, the CR formulation of the first release component mostly, generally, or entirely is composed of one or more CR polymers, which can be, e.g., pH-independent polymers or polymers that are otherwise susceptible to dissolution under gastric acid dissolution conditions. In certain aspects, about 25%-about 45%, e.g., ˜25%-˜40%, ˜25%-˜35%, ˜25%-˜30%, or ˜30%-˜45%, ˜35%-˜45%, or ˜40%-˜45%, such as, e.g., ˜30%-˜40% of a gastric acid dissolution susceptible component present in a composition is composed of a release retardant polymer component.

Aspects relating to such polymers are described in the “General Characteristics” section of this Detailed Description. Additionally, characteristics of such polymers (and the relation of such a polymer component to other elements of the composition such as the NCDC(s)) are contained in, e.g., various aspects selected from aspects 1-90 in the Exemplary Aspects of the Invention section.

In aspects, the polymer component of the first release component is mostly, generally only, or only composed of cellulosic controlled release polymer(s), such as, e.g., HPMC, hydroxypropyl cellulose, hydroxyethyl cellulose, and methylcellulose. In aspects, some of the CR polymer matrix/formulation also or alternatively includes one or more pharmaceutically acceptable alginates, Carbopol composition(s), or gelatin. In aspects, the CR polymer component is mostly, generally, or entirely hydrophobic. In aspects, the CR polymer component also or alternatively comprises an effective amount of a lipophilic component, such as carnauba wax, cetyl alcohol, hydrogenated castor oil, microcrystalline waxes, ethyl cellulose (EC), stearic acid, or polyvinyl acetate (PVAc). In aspects, the CR formulation of the first release component comprises a pH-dependent or pH-independent polymethacrylate. In aspects, the matrix/formulation comprises components(s) that aid in compressibility, such as PVAc or MCC/SMCC. In aspects, some, most, or generally all of the controlled release polymer component of first release component formulations or dosage forms is composed of a hydroxypropyl methylcellulose. In aspects, most, generally all, or all of the polymers in the first release component are highly hydrophilic. Additional relevant principles, compositions, methods, etc., applicable to such formulations are known in the art with respect to such formulations (see, e.g., Sowjanya, M. et al. (2017). Research Journal of Pharmacy and Technology. 10. 903. 10.5958/0974-360X.2017.00168.8 and Sarah J. Trenfield, et al., Chapter 6—Modified drug release: Current strategies and novel technologies for oral drug delivery, Editor(s): João Pedro Martins, Hélder A. Santos, Nanotechnology for Oral Drug Delivery, Academic Press, 2020, Pages 177-197, SBN 9780128180389, doi.org/10.1016/B978-0-12-818038-9.00006-5.

### Dissolution Profile of First Release Tablets

In aspects, contacting the one or more first release tablets with a dissolution medium having a pH of between about 1 and 1.5 (which mimics the acidic environment of the stomach), such as a pH of about 1.1, such as ˜1.2, ˜1.3, or a pH of ˜1.4 and at a temperature of about physiological temperature, i.e. ˜37° C., and under moderate stirring, e.g., via use of a USP apparatus type II system (Agilent) provides for a dissolution profile comprising one or more of the characteristics described herein. In aspects, generally here when referring to dissolution conditions, the conditions are dissolution testing conditions comprising a stated pH of a dissolution medium maintained at about 37° C.+/−5° C. under constant stirring, such as, e.g., stirring at between about 40-about 60 rpm, such as, e.g., between about 45-55 rpm, such as, e.g., 50 rpm, using dissolution equipment standard in the art, such as, e.g., USP apparatus type II system (Agilent).

In aspects, between about 10% and 30%, such as between about 12% and 28%, such as between about 15% and 25%, or e.g., between about 17% and 22% (e.g., ˜15%-˜24%, ˜15%-˜22%, ˜15%-˜20%, ˜15%-˜18%, ˜16%-˜25%, ˜18%-˜25%, ˜20%-˜25%, or ˜22%-˜25%) of the contents of a first release tablet are dissolved within about 30 minutes after administration.

In another aspect, the first release tablet component presents a dissolution profile wherein between about 30% and 60%, e.g., ˜30%-˜55%, ˜30%-˜50%, ˜30%-˜45%, ˜30%-˜40%, or ˜35%-˜60%, ˜40%-˜60%, ˜45%-˜60%, ˜50%-˜60%, or, e.g., ˜55%-˜60%, such as between about 35% and 55%, such as between about 40% and 50%, such as between about 42% and 48% of the are dissolved within about 1 hour after administration.

In another aspect, the first release tablet component presents a dissolution profile wherein between about 50% and 80%, e.g., ˜55%-˜80%, ˜60%-˜80%, ˜65%-˜80%, ˜70%-˜80%, or ˜75%-˜80%, e.g., ˜50%-˜75%, ˜50%-˜70%, ˜50%-˜65%, ˜50%-˜60%, ˜50%-˜55%, such as between about 55% and 75%, such as between about 60% and 70% of the contents are dissolved within about 2 hours after administration.

In yet another aspect, the first release tablet component presents a dissolution profile wherein between about 75% and 100%, e.g., ˜75%-˜95%, e.g., ˜75%-˜90%, ˜75%-˜85%, ˜75%-˜80%, e.g., e.g., ˜80%-˜100%, e.g., ˜85%-˜100%, e.g., ˜90%-˜100%, or, e.g., e.g., ˜95%-˜100%, such as between about 80% and about 95%, between about 85% and 98%, such as between about 89% and 96%, such as between about 90% and 95% of the contents are dissolved within about 6 hours after administration.

In another aspect, the first release tablet component presents a dissolution profile wherein between about 98% and 100%, e.g., ˜98%, ˜98.5%, ˜99%, ˜99.5%, or, e.g., ˜100% of the contents are dissolved by about 8 hours after administration.

The Exemplary Aspects of the Invention herein contain numerous aspects that characterize exemplary dissolution profile characteristics of the first release component (e.g., a GADSC) and components thereof (e.g., dosage forms that make up such a component). Such aspects can be combined with any of the descriptions provided in this section, in whole or in part, to reflect the full range of such embodiments envisioned by the inventors.

In another aspect, contacting the one or more first release tablets with a dissolution medium having a pH of between about 5 and 7, such as between ˜5.5-7, ˜6-7, or ˜6.5-7 (which mimics the later sections of the intestine(s), such as about 6.1, such as about 6.2, such as about 6.3, such as about 6.4, such as about 6.5, such as about 6.6, such as about 6.7, such as about 6.8, or such as about 6.9, and at a temperature of about normal physiological/body temperature, i.e. 37° C., and (in aspects) under moderate stirring, provides for a dissolution profile as described herein.

In aspects, the first release tablet component presents a dissolution profile wherein between about 5% and 25%, e.g., ˜5%-˜20%, e.g., ˜5%-˜15%, e.g., ˜5%-˜10%, e.g., e.g., ˜10%-˜25%, e.g., ˜15%-˜25%, or e.g., ˜20%-˜25%, such as between about 8% and 22%, or such as between about 10% and 20% or ˜12% to about 18% of the contents are dissolved within about 30 minutes after contact with such a dissolution solution under such conditions. In another aspect the first release tablet presents a dissolution profile wherein between about 10% and 40%, e.g., ˜15%-˜40%, ˜20%-˜40%, ˜25%-˜40%, ˜30%-˜40%, ˜35%-˜40%, or, e.g., ˜10%-˜35%, ˜10%-˜30%, ˜10%-˜25%, ˜10%-˜20%, ˜10%-˜15%, such as between about 15% and 35%, such as between about 20% and 30%, such as between about 22% and 28% of the tablet are dissolved within about 1 hour after contact with such a first dissolution solution under such conditions. In another aspect, the first release tablet presents a dissolution profile wherein between about 30% and 60%, e.g., ˜35%-˜60%, ˜40%-˜60%, ˜45%-˜60%, ˜50%-˜60%, ˜55%-˜60%, e.g., ˜30%-˜55%, ˜30%-˜50%, ˜30%-˜45%, ˜30%-˜40%, ˜30%-˜35%, such as between about 35% and 55%, such as between about 40% and 50% of the contents are dissolved within about 2 hours after contact with such a dissolution solution under such conditions. In another aspect, the first release tablet component presents a dissolution profile wherein between about 60% and 90%, e.g., ˜65%-90%, ˜70%-90%, ˜75%-90%, ˜80%-90%, ˜85%-90%, or, e.g., ˜60%-85%, ˜60%-80%, ˜60%-75%, ˜60%-70%, ˜60%-65%, such as between about 65% and 85%, such as between about 70% and 80%, or, e.g., 72% to about 78% of the contents are dissolved within about 6 hours after contact with such a solution under such conditions. In yet another aspect, the first release tablet component presents a dissolution profile wherein between about 70% and about 98%, e.g., ˜70%-95%, ˜70%-90%, ˜70%-85%, ˜70%-80%, ˜70%-75%, e.g., ˜75%-98%, ˜80%-98%, ˜85%-98%, ˜90%-98%, ˜95%-98%, such as between about 75% and 93%, such as between about 80% and 87% of the contents are dissolved within about 8 hours after contact with such a solution under such conditions. In another aspect, the first release tablet component presents a dissolution profile wherein between about 70% and 98%, such as between about 75% and 93%, such as between about 80% and 87% of the contents are dissolved within about 8 hours after contact with such a first dissolution solution under such conditions. In another aspect, the first release tablet component presents a dissolution profile wherein between about 80% and 98%, e.g., ˜80%-95%, ˜80%-˜90%, or, e.g., ˜85%-˜98%, ˜90%-˜98%, or ˜95%-˜98%, such as between about 82% and 96%, such as between about 84% and 95%, such as between about 87% and 92% of the contents are dissolved within about 10 hours after contact with such a dissolution solution under such conditions (i.e., after remaining in contact with such a solution under such conditions for about 10 hours). In an aspect, the first release tablet component presents a dissolution profile wherein between about 80% and 98%, e.g., ˜82%-98%, ˜84%-98%, ˜86%-98%, ˜88%-98%, ˜90%-98%, ˜92%-98%, ˜94%-98%, or ˜96%-98%, as in, e.g., ˜80%-˜96%, ˜80%-˜94%, ˜80%-˜92%, ˜80%-˜90%, ˜80%-˜88%, ˜80%-˜86%, ˜80%-˜84%, or ˜80%-˜82%, such as between about 82% and 96%, such as between about 84% and 95%, such as between about 86% and 93% of the contents are dissolved within about 12 hours after contact with such a solution under such conditions.

### C. Second Release Component

In aspects, compositions of the invention include a second release component. The second release component comprises a controlled release formulation that comprises a detectably or significantly different composition and is significantly different in one or more performance characteristics from the first release component in one or more respects, typically in respect of the release of NCDCs from the second release component as compared to the first release component, e.g., the timing, amount(s), or both of such release, post-administration of the composition. In aspects, the second release component comprises a part (e.g., a portion) of the total therapeutically effective amount of NCDC(s), e.g., nicorandil, present in the composition. In aspects, the amount of NCDC(s) present in the first part (or portion) of the total therapeutically effective amount of NCDC(s), e.g., nicorandil, present in the composition varies by no more than about 10% (e.g., by no more than ˜9%, ˜8%, ˜7%, ˜6%, ˜5%, ˜4%, ˜3%, ˜2%, or, e.g., by no more than about 1%) from the amount of NCDC(s), e.g., nicorandil, present in the second part (or portion) of the total therapeutically effective amount of NCDC(s), e.g., nicorandil, in the composition. In aspects, the second release component can be categorized as a controlled release gastric acid dissolution resistant component (GADRC). In such a respect, the second release component can comprise an effective amount of a pH responsive component that significantly retards/impedes/reduces the release of NCDCs from the second release component under gastric acid dissolution conditions. In aspects, a GADRC comprise a release retardant polymer component, a pH responsive release resistant polymer component, or both. In aspects, the second release component and first release component are contained in a delivery facilitating agent/shell, e.g., a capsule. In aspects, the second release component is composed of 2-8, 3-8, 3-6, 3-4, 3, 4, 5, or 6 solid dosage forms (e.g., tablets). In aspects, the second release component comprises about 33% to 66% (e.g., ˜35%-66%, ˜40%-66%, ˜45%-66%, ˜50%-66%, ˜55%-66%, or ˜60%-66%, e.g., ˜33%-˜65%, ˜33%-˜60%, ˜33%-˜55%, ˜33%-˜50%, ˜33%-˜45%, or ˜33%-˜40%, e.g., ˜35%-˜60%, about 40-60%, 45-65%, or about 50% of the controlled release formulation of the composition. In aspects, the second release component comprise an equal number of solid dosage forms as the first release component. In aspects, the second release component comprises a different number of solid dosage forms than the first release component.

Second release component dosage form(s) can comprise any suitable GADR component, such as a pH responsive polymer component, formulated in any suitable manner. In aspects, some, most, generally all, or all of the GADR component is embodied in a coating that is applied to a core of the solid dosage form(s) that make up the second release component. In aspects, the coating is the only difference between the first release component and second release component. In exemplary aspects, second release tablets comprise one or more pharmaceutically acceptable coatings. In aspects, second release tablets can comprise a core composition corresponding to the composition of any one of the first release dosage forms/tablets described in the preceding sections of this disclosure plus one or more pharmaceutically acceptable coatings. In aspects, the coating comprises one or more pharmaceutically acceptable coating materials such as cellulose ether(s), vinyl polymer(s) (including vinyl derivative(s)), glycol(s), acrylic acid polymer(s), other carbohydrate(s), and combinations of some or all thereof.

In aspects, the one or more coatings can be a coating characterizable a pH sensitive/delayed release coating (as compared to, e.g., release form the first release dosage form). In aspects. Such one or more coatings also can also or alternatively be characterizable as an enteric coating(s). In aspects, such a coating can be adapted for enteric release, e.g., can detectably or significantly improve/promote a release of NCDC(s) allowing for such a tablet to pass through the stomach without substantial dissolution (e.g., as exemplified in the Figures and Exemplary Aspects of the Invention). In aspects, the second release component releases most, generally all, substantially all, or all of the NCDCs upon contact with a dissolution media of about pH of 5.5 or higher (e.g., about 6.8), such as may be encountered in the mid-to-upper portions of the intestine (e.g., the duodenum, jejunum, ileum, etc.).

In aspects, the second release component comprises an effective amount of one or more pH-dependent excipients, such as methacrylate polymers, ethyl cellulose, or the like, to provide gastro-resistant functionality. In aspects, coatings used for second release component dosage forms also or alternatively comprise CAP (cellulose acetate phthalate), CAT (cellulose acetate trimellitate), PVAP (Polyvinyl acetate phthalate), and HPMCP (HPMC phthalate), Hydroxyl propyl methyl cellulose acetate succinate (HPMCAS) fatty acids, waxes, shellac, plastics, or plant fibers. In aspects, the coating of a second release component or second release component dosage form comprises an effective amount of a plasticizer component, comprising or mostly comprising, or at least generally consisting of e.g., water-soluble and insoluble plasticizers, such as diethyl phthalate (DEP), tributyl citrate (TBC), triethyl citrate (TEC), tributyrin, and triacetin plasticizers. According to aspects, some, most, generally all, or all of the pH responsive polymer(s) of a second release formulation/dosage form are composed of one or more suitable methacrylic acid copolymers. These enteric polymers are marketed most notably by Evonik (formerly known as Röhm GmbH) under the proprietary Eudragit brand name. Methacrylic acid methylmethacrylate copolymers (Eudragit L and S), and methacrylic acid ethyl acrylate copolymer (Eudragit L30D) are examples of polymers known to target release in different parts of the intestine due to their primary dissolution pH characteristics (e.g., Eudragit L 30 D-55 and L 100 D-55 having a primary dissolution pH of about 5.5; Eudragit L 100 and L 12.5 having a primary dissolution pH range of about 6-about 7; and Eudragit S 100, S 12.5 and ES 30D having a primary dissolution pH of about 7. Of course, readers will understand these properties are exemplary and can be applied to other suitable types of pH responsive polymers. Examples of such compositions and related principles/methods as described in this section are further provided in, e.g., Patil G. et al. Review on Enteric Coated Polymers, Research Journal of Pharmaceutical Dosage Forms and Technology; Raipur Vol. 13, Iss. 1 (January-March 2021): 82-85. DOI:10.5958/0975-4377.2021.00015.X and Devesh Kapoor, et al. Chapter 14—Coating technologies in pharmaceutical product development, In Advances in Pharmaceutical Product Development and Research, Drug Delivery Systems, Academic Press, 2020, Pages 665-719, ISBN 9780128144879, doi.org/10.1016/B978-0-12-814487-9.00014-4.

### i. pH Sensitive Polymer(s)

In aspects, a coating can comprise one or more pharmaceutically acceptable polymers. In aspects, the polymer can be an amorphous polymer. In aspects, the polymer can be a polymer designed for enteric release, e.g., by significantly promoting second release such as release in the GI track past the stomach (in the intestines). In aspects, a polymer of a coating can be, mostly comprise, or generally comprise a pH dependent polymer comprising carboxylic acid groups. In aspects, such a polymer can remain un-ionized in low pH conditions, e.g., in the environment of the stomach, and become ionized with increasing pH, such as, e.g., as the polymer is exposed to the higher pH of the small intestine. In aspects, the pH dependent polymer is configured to perform according to changes in pH. In aspects, the pH dependent polymer does not begin to significantly dissolve (e.g., does not demonstrate substantial dissolution) until it is subjected to a pH of at least 5.5, e.g., until it is subjected to a pH of between about 5-7.5, such as, e.g., ˜5-7, ˜5-6.5, ˜5-6, or ˜5.5-7.2, ˜5.5-7, ˜5-6.5, ˜5.5-6, or ˜6-7. In aspects, a second release formulation can be categorized/described as a GADRC.

In aspects, the pH sensitive polymer component of a GADRC/second release formulation can be, generally comprise/consist of, essentially consist of, or primarily comprise any pharmaceutically acceptable pH sensitive polymer such as polyacids, polybases, and natural polymers, or combination thereof. In further aspects, natural polymers that are pH sensitive also can include chitosan, hyaluronic acid, and dextran, or a combination thereof.

In certain aspects, polymers that impart GADR/pH-responsive functions to the second release formulation or elements thereof can be pH-dependent cellulose derivatives. In aspects, cellulose derivatives used as enteric coating polymers can include cellulose acetate phthalate (CAP), cellulose acetate trimelitate (CAT), hydroxypropylmethyl cellulose phthalate (HPMCP), carboxymethylethyl cellulose (CMEC), hydroxypropylmethyl cellulose acetate succinate (HPMCAS), etc. In some respects, suitable polymers for use in a coating are cationic synthetic polymers based on methacrylate groups (e.g., characterizable as methacrylate polymers). In aspects, such polymers are Eudragit® polymers, such as, e.g., Eudragit® E, Eudragit® L, Eudragit® S, Eudragit® RL, Eudragit® RS, Eudragit® FS, Eudragit® NM e.g., Eudragit® L30 D-55, Eudragit® FS 30 D, Eudragit® FL 30 D-55, etc. In specific aspects, a coating can comprise an aqueous dispersion of an anionic copolymer based on methacrylic acid and ethyl acrylate; e.g., a polymer having methacrylic acid as a functional group. In aspects, such a polymer can have free carboxylic groups in a ratio of about 1:1 with the ester groups. In aspects, a suitable polymer present in a coating is Eudragit® L30 D-55.

In aspects, a polymer can be present an amount of between about 1 mg-about 15 mg per tablet, as in, e.g., ˜1-˜14 mg, ˜1-˜12 mg, ˜1-˜10 mg, ˜1-˜8 mg, ˜1-˜6 mg, ˜1-˜4 mg, ˜1-˜2 mg, e.g., ˜2-˜15 mg, ˜4-˜15 mg, ˜6-˜15 mg, ˜8-˜15 mg, ˜10-˜15 mg, or ˜12-˜15 mg, e.g., in an amount between ˜2 mg-˜14 mg, ˜3 mg-˜13 mg, ˜4 mg-˜12 mg, ˜5 mg-˜11 mg, or, e.g., in an amount of between ˜6 mg-˜10 mg, such as, e.g., between ˜6-˜9 mg, ˜6-˜8 mg, ˜6-˜7 mg, or, e.g., ˜7 mg-˜10 mg, ˜8 mg-˜10 mg, or, e.g., ˜9 mg-˜10 mg per tablet. In aspects, a polymer can be present in an amount of about 6.66 mg-about 10 mg per tablet. In aspects, some coated tablets, most coated tablets, or each coated tablet comprises between about 5-12, e.g., about 6-11, such as about 6.5-10.5 mg or 6.66-about 10 mg of Eudragit® L30 D-55.

In aspects, the pH sensitive polymer(s) causes the second release tablets to produce a dissolution profile wherein the tablets do not begin to detectably or significantly dissolve before about 10 hours, such as before about 11 hours, such as before about 12 hours, such as before about 13 hours, or such as before about 14 hours, when subjected to a pH of between about 1 and 1.5 (which mimics the acidic environment of the stomach), such as a pH of about 1.1, e.g., ˜1.2, ˜1.3, or a pH of ˜1.4 (e.g., under the dissolution conditions described above in terms of stirring and temperature).

The Exemplary Aspects of the Invention include aspects that speak to exemplary characteristics and other facets of second release components/GADRCs and elements thereof (such as GADRC dosage forms of a composition). Such aspects, include, aspects relating to dissolution profiles of a GADRC in pH 6.8 media and pH 1.2 media and various exemplary components that can make up such a component and the relationship of such elements/ingredients. Such aspects can be combined with the disclosure provided in this and related sections of the disclosure to arrive at a more complete understanding of the scope of the invention.

In aspects, a coating can comprise one or more compounds which act to increase the coverage of the coating, create a barrier effect, serve as a lubricant, serve as a glidant, improve flow of coating components, improve upon the smoothness of the coating upon application to a tablet, or any combination of any or all thereof. In certain aspects, such a compound can be any one or more pharmaceutically acceptable compounds providing any one or more of such effects. In certain aspects, such a compound can be, e.g., a starch, silicas (e.g., colloidal silica, silicates, etc.), glyceryl monostearate, magnesium carbonate, magnesium stearate hydrated magnesium silicate (MgSiO₃), often referred to as talc, etc. In aspects, a coating can comprise no such component (e.g., the coating lacks a glidant). In aspects, a coating comprises a plasticizer (e.g., triethyl citrate (TEC)), an anti-tacking agent (e.g., mono- or di-glycerides), and an emulsifier/stabilizer (e.g., a polysorbate, such as polysorbate 80).

In aspects, a coating can comprise between about 0-about 5 mg of a compound characterizable as a glidant or a compound providing such functionality as described in this paragraph per tablet, such as, e.g., between 0 mg-˜4.5 mg, 0 mg-˜4 mg, 0 mg-˜3.5 mg, 0 mg-˜3 mg, or, e.g., 0 mg-˜2.75 mg of such a compound per tablet, e.g., ˜0.1-5 mg, ˜0.5-5 mg, ˜1-5 mg, ˜1.5-5 mg, ˜2-5 mg, ˜2.5-5 mg, ˜3-5 mg, ˜3.5-5 mg, or ˜4-5 mg, such as, e.g., ˜0.05 mg-˜4 mg, ˜0.1 mg-˜3 mg, ˜0.2 mg-˜2.9 mg, ˜0.3-˜2.8 mg, ˜0.4-˜2.7 mg, ˜0.5-˜2.7 mg, ˜0.6-˜2.7 mg, ˜0.7-˜2.7 mg, ˜0.8-˜2.7 mg, ˜0.9 mg-˜2.7 mg, ˜1 mg-˜2.7 mg, ˜1.2 mg-˜2.7 mg, ˜1.4 mg-˜2.7 mg, ˜1.6 mg-˜2.7 mg, ˜1.8 mg-˜2.7 mg, ˜2 mg-˜2.7 mg, ˜2.1 mg-˜2.7 mg, ˜2.2 mg-˜2.7 mg, ˜2.3 mg-˜2.7 mg, ˜2.4 mg-˜2.7 mg, ˜2.5 mg-˜2.7 mg, or, e.g., ˜2.6 mg-˜2.7 mg per tablet. In aspects, a tablet can comprise a coating comprising talc in an amount up to about 2.66 mg per tablet. In aspects, a tablet can comprise a coating not comprising talc.

In aspects, a coating can comprise one or more compounds which aid in providing a uniform, e.g., smooth, and consistently resistant enteric film on a coated tablet. In aspects, such a compound is characterizable as a plasticizer. In aspects, a coating can comprise any one or more pharmaceutically acceptable plasticizers or compounds providing such function as described in this paragraph. In aspects, such compounds can be, e.g., diethyl phthalate, glyceryl triacetate, glyceryl monocaprylate, triethyl citrate, etc. As stated above, in aspects, hydrophilic cellulose ether derivatives, e.g., HPMC, can be used with enteric polymer for improving the film forming and plasticity of the main enteric polymer. In aspects, polymer(s) in coating(s) exhibit (a) film forming properties, (b) polymer-to-polymer adhesion with other enteric coating polymers especially with cellulose ester derivatives such as cellulose acetate phthalate (CAP), hydroxypropylmethyl cellulose phthalate (HPMCP), hydroxypropylmethyl cellulose acetate succinate HPMCAS, carboxymethylethyl cellulose (CMEC), and cellulose acetate trimelitate (CAT), or (c) both (a) and (b) to a degree that is significantly similar to HPMC.

In aspects, a coating can comprise between about 0.1-about 1 mg of a compound having one or more of the characteristics of the above paragraph, such as in aspects characterizable as a plasticizer, such as, e.g., between ˜0.1-˜0.8 mg, ˜0.1-˜0.6 mg, ˜0.1-˜0.4 mg, ˜0.1-˜0.2 mg, e.g., ˜0.2-˜1 mg, ˜0.4-˜1 mg, ˜0.6-˜1 mg, or ˜0.8-˜1 mg, e.g., ˜0.1 mg-˜0.9 mg, ˜0.2 mg-˜0.8 mg, ˜0.3 mg-˜0.7 mg, ˜0.4 mg-˜0.7 mg, ˜0.5 mg-˜0.7 mg, or, e.g., between ˜0.6 mg-˜0.7 mg of such a compound per tablet. In specific aspects, a coating can comprise about 0.66 mg of triethylcitrate.

In aspects, about 25% to about 45%, e.g., ˜25%-˜40%, ˜25%-˜35%, ˜25%-˜30%, e.g., ˜30%-˜45%, ˜35%-˜45% or ˜40%-˜45%, e.g., ˜30%-˜40% of the gastric acid dissolution resistant component is composed of a release retardant polymer component.

In aspects, a second release component comprises a plurality of second release dosage forms (e.g., 2-8, 2-6, 3-6, or 3-4 second release tablets). In aspects, ˜25%-˜80%, such as about 25%, about 30%, about 33%, about 35%, about 40%, about 45%, about 50%, about 55%, about 60%, about 66%, about 70%, or about 75%, of the total number of solid dosage forms/units/components in a composition are second release dosage forms/units.

### Dissolution Profile of Second Release Component/Second Release Tablets

In aspects, contacting the second release component, such as one or more second release solid dosage forms (e.g., second release tablets or other solid orally administrable dosage form(s) such as those described above) with a dissolution medium having a pH of between about 1 and 1.5 (which mimics the acidic environment of the stomach), such as about 1.1, such as about 1.2, such as about 1.3, or such as a pH of about 1.4, in aspects at a temperature of about normal body/physiological temperature, i.e., about 37° C. (or at room temperature, or both), and, in aspects, under moderate stirring (e.g., under dissolution testing conditions exemplified above) provides for one or more characteristic(s) of a dissolution profile described herein (e.g., in the Exemplary Aspects of the Invention). In an aspect, the second release tablets show little to no detectable or significant release within this pH range (pH 1-pH 1.5) after exposure to such a pH for a period of up to about 6 hours, ˜7 hours, ˜8 hours, ˜9 hours, ˜10 hours, ˜11 hours, ˜12 hours, ˜13 hours, ˜14 hours, ˜15 hours, ˜16 hours, ˜17 hours, or, e.g., ˜18 hours (hrs.). In aspects, less than 5% of the contents of the second release tablets are dissolved prior to about 14 hours after administration, such as less than ˜4%, such as less than ˜3%, such as less than ˜2%, such as less than ˜1%, such as less than ˜0.9%, ˜0.8%, ˜0.7%, ˜0.6%, ˜0.5%, ˜0.4%, ˜0.3%, ˜0.2%, or less than ˜0.1% of the contents of the second release tablets are dissolved prior to about 14 hours after administration. In further aspects, the amount of NCDC(s), e.g., nicorandil, dissolved prior to about 14 hours is undetectable using standard analytical techniques.

In aspects, contacting the one or more second release tablets with a dissolution medium having a pH of about 5-7, e.g., ˜5-6.8, ˜5-6.6, ˜5-6.4, ˜5-6.2, ˜5-6, or ˜5.2-7, ˜5.4-7, ˜5.6-7, or ˜5.8-7 e.g., about 5.5-7.5, ˜5.5-7.2, ˜5-7.2, ˜6.5-7, or ˜6-7 (which mimics the later sections of the intestines), such as about 6.1, such as about 6.2, such as about 6.3, such as about 6.4, such as about 6.5, such as about 6.6, such as about 6.7, such as about 6.8, or such as about 6.9, and at a temperature of about normal body temperature, i.e. 37° C., and under moderate stirring for about 2 hours results in the release of between about 20%-about 50%, e.g., ˜25%-˜50%, e.g., ˜30%-˜50%, e.g., ˜35%-˜50%, e.g., ˜40%-˜50%, e.g., ˜45%-˜50%, e.g., e.g., ˜20%-˜45%, ˜20%-˜40%, ˜20%-˜35%, ˜20%-˜30%, ˜20%-˜25%, such as between about 25% and 45%, such as between about 30% and 40% or between about 32% and about 38% of the NCDC(s), e.g., nicorandil, being released from the second release tablets. In another aspect, at the same dissolution conditions, about 90% (e.g., about 85% to about 95%) of the contents of the second release tablets are released, within between about 10 hours and 11 hours after administration.

According to aspects, a GADRC releases no more than ˜20% of the one or more nicorandil compounds contained in the GADRC at 2 hours of contact with a dissolution media having a pH of 1.2 (such as releasing no more than ˜15%, no more than ˜12.5%, no more than ˜10%, no more than ˜7.5%, or no more than ˜5%, e.g. about 1-20%, ˜1-15%, ˜1-10%, ˜1-5%, ˜2-20%, ˜5-20%, ˜10-20%, or ˜15-20%, e.g., ˜2-15%, ˜2-10%, ˜2-5%, ˜3-15%, ˜3-12%, ˜5- 20%, ˜5-15%, or ˜5-10% of the nicorandil compound(s) initially contained in the GADRC under such a condition(s). According to aspects, a GADRC releases no more than ˜20% of the one or more nicorandil compounds contained in the GADRC at 6 hours of contact with a dissolution media having a pH of 1.2 (such as releasing no more than ˜15%, no more than ˜12.5%, no more than ˜10%, no more than ˜7.5%, or no more than ˜5%, e.g. about 1-20%, ˜1-15%, ˜1-10%, ˜1-5%, ˜2-20%, ˜5-20%, ˜10-20%, or ˜15-20%, e.g., ˜2-15%, ˜2-10%, ˜2-5%, ˜3-15%, ˜3-12%, ˜5-20%, ˜5-15%, or ˜5-10% of the nicorandil compound(s) initially contained in the GADRC under such a condition(s).

In aspects, a GADRC comprises an amount of one or more pH responsive polymers that is effective to release at least about 33% (e.g., ≥˜35%, ≥˜40%, ≥˜45%, or ≥˜50%, of the one or more nicorandil compounds initially present in the pH responsive dosage form 5 hours or 6 hours after contact with a dissolution media having a pH of about 6.8 and no more than about 20% (e.g., ≤˜18%, ≤˜15%, ≤˜12%, ≤˜10%) of the one or more nicorandil compounds initially present in the pH responsive dosage form 5 hours after contact with a dissolution media having a pH of about 1.2.

According to aspects, when maintained in contact with a dissolution media of pH 6.8 the amount of the one or more nicorandil compounds being released from a GADRC is in accordance mostly, generally, or entirely with the following release profile: (1) about 0% to about 8.5%, such as about 3% to about 8%, such as about 7% of the NCDC(s) being released from the GADRC after 0.25 hours in the dissolution media; (2) about 15.5% to about 29%, such as about 19% to about 25%, such as about 22.25% of the NCDC(s)) being released at 1 hour in the dissolution media; (3) about 27% to about 50%, such as about 33% to about 43%, such as about 38.3% of the NCDC(s) being released at 2 hours in the dissolution media; (4) about 65-80%, e.g., about 67-77%, such as about 72.5% of the NCDC(s) being released at 6 hours in the dissolution media; (5) about 75% to about 87.5%, such as 78% to about 85%, such as about 81.5% of the NCDC(s) being released at 8 hours contact with the dissolution media, and (6) about 83.5% to about 98% or about 100%, such as about 87% to about 94%, e.g., about 90.5% of the NCDC(s) in the GADSC being released at 14 hours of contact with the dissolution media.

According to aspects, when maintained in contact with a dissolution media of pH 6.8 under typical physiological conditions such as those described herein, the amount of the one or more nicorandil compounds released from a GADRC is about 0% to about 8.5%, e.g., ˜0%-˜7.5%, ˜0%-˜7%, ˜0%-˜6.5%, ˜0%-˜6%, ˜0%-˜5.5%, ˜0%-˜5%, ˜0%-˜4.5%, ˜0%-˜4%, ˜0%-˜3.5%, ˜0%-˜3%, ˜0%-˜2.5%, ˜0%-˜2%, ˜0%-˜1.5%, or ˜0%-˜1%, e.g., 0.5%-˜8.5%, 1%-˜8.5%, 1.5%-˜8.5%, 2%-˜8.5%, 2.5%-˜8.5%, 3%-˜8.5%, 3.5%-˜8.5%, 4%-˜8.5%, 4.5%-˜8.5%, 5%-˜8.5%, 5.5%-˜8.5%, 6%-˜8.5%, 6.5%-˜8.5%, 7%-˜8.5%, 7.5%-˜8.5%, or 8%-˜8.5%, as in, e.g., ˜1%-˜8%, or ˜2%-˜7%, ˜3%-˜6%, or, e.g., ˜4%-˜8%, ˜5%-˜8%, or 6%-˜8%, such as about 3% to about 8%, such as about 7% after 0.25 hours in the dissolution media.

According to aspects, when maintained in contact with a dissolution media of pH 6.8 under typical physiological conditions such as those described herein, the amount of the one or more nicorandil compounds released from a GADRC is about 15.5% to about 29%, e.g., ˜17%-˜29%, ˜19%-˜29%, ˜21%-˜29%, ˜23%-˜29%, or ˜25%-˜29%, e.g., ˜15.5%-˜27%, ˜15.5%-˜25%, ˜15.5%-˜23%, ˜15.5%-˜21%, ˜15.5%-˜19%, such as about 19% to about 25%, such as about 22.25% after 1 hour in the dissolution media.

According to aspects, when maintained in contact with a dissolution media of pH 6.8 under typical physiological conditions such as those described herein, the amount of the one or more nicorandil compounds released from a GADRC is about 27% to about 50%, e.g., ˜30%-˜50%, ˜35%-˜50%, ˜40%-˜50%, ˜45%-˜50%, e.g., ˜27%-˜45%, ˜27%-˜40%, ˜27%-˜35%, ˜27%-˜30%, as in, e.g., ˜30%-˜45%, such as about 33% to about 43%, such as about 38.3% of the NCDC(s) at 2 hours in the dissolution media.

According to aspects, when maintained in contact with a dissolution media of pH 6.8 under typical physiological conditions such as those described herein, the amount of the one or more nicorandil compounds released from a GADRC is about 65-80%, e.g., ˜70%-˜80%, ˜75%-˜80%, or ˜65%-˜75% or ˜65%-˜70%, such as, e.g., about 67-77%, or about 69% to about 75%, or, e.g., about 72.5% at 6 hours in the dissolution media.

According to aspects, when maintained in contact with a dissolution media of pH 6.8 under typical physiological conditions such as those described herein, the amount of the one or more nicorandil compounds released from a GADRC is about 75% to about 87.5%, e.g., ˜75%-˜85%, ˜75%-˜82%, ˜75%-˜80%, or ˜77%-˜87.5%, ˜80%-˜87.5%, ˜82%-˜87.5%, or ˜85%-˜87.5%, such as 78% to about 85%, ˜80%-˜83%, or such as about 81.5% of the NCDC(s) being released at 8 hours contact with the dissolution media.

According to aspects, when maintained in contact with a dissolution media of pH 6.8 under typical physiological conditions such as those described herein, the amount of the one or more nicorandil compounds released from a GADRC is about 83.5% to about 98% or about 100%, as in, e.g., ˜83.5%-˜95%, ˜83.5%-˜90%, ˜83.5%-˜85%, e.g., ˜85%-˜100%, ˜90%-˜100%, or ˜95%-˜100%, such as about ˜85%-˜96%, about 87% to about 94%, or, e.g., about 90.5% of the NCDC(s) in the GADSC being released at 14 hours of contact with the dissolution media.

In aspects, there may be between about 1-4, ˜2-4, ˜1-3, ˜2-3, ˜3, or ˜2 second release tablet components/units/dosage forms, such as two components, or such as ˜3 components in a single administration form (e.g., in a single delivery facilitating agent/shell, e.g., capsule). In aspects, there may be between about 1-4, ˜2-4, ˜1-3, ˜2-3, ˜3, or ˜2 second release tablet components/units/dosage forms, in a single administration form (e.g., a single delivery facilitating agent/shell, e.g., capsule) along with between about 1-4, ˜2-4, ˜1-3, ˜2-3, ˜3, or ˜2 first release tablet components/units/dosage forms (these can be present in the same amount or in different amounts such as, e.g., 1 second release tablet to 3 first release tablets or 3 second release tablets and 1 first release tablet, etc.). In aspects, the number of first release tablet components/units/dosage forms present in a single administration form (e.g., a single delivery facilitating agent/shell, e.g., capsule) can be more than the number of second release tablet components within the single delivery facilitating agent/shell, e.g., capsule. In aspects, the number of first release tablet components/units/dosage forms present in a single administration form (e.g., a single delivery facilitating agent/shell, e.g., capsule) can be less than the number of second release tablet components within the single delivery facilitating agent/shell, e.g., capsule. In aspects, the number of first release tablet components/units/dosage forms present in a single administration form (e.g., a single delivery facilitating agent/shell, e.g., capsule) can be the same as the number of second release tablet components within the single delivery facilitating agent/shell, e.g., capsule. In specific aspects, two first release tablets and two second release tablets are provided within a single capsule. In aspects, the single capsule represents one single administrative dose. In aspects, only one single administrative dose is provided per 24-hour period. In aspects, the first and second release tablets within the single administrative dose collectively comprise a sufficient amount of nicorandil to detectably or significantly affect a therapeutic effect on a target condition.

In aspects, any one, some, or all of the aspects herein characterized by inclusion of release rates implicitly disclose an aspect characterized by also or alternatively releasing a corresponding approximately relative overall amount of nicorandil compound in the applicable/given period. For example, if an aspect explicitly describes a second release rate being 1.25×, or, e.g., 25% greater, than a first release rate, such aspect will be understood to implicitly disclose alternative aspects where also or alternatively, the amount of nicorandil released in the second period is characterized by an approximately corresponding overall increase in amount of released nicorandil (e.g., the amount of nicorandil released in the second period would also or alternatively in the exemplified aspect be about 1.25× the amount released in the first period).

In other aspects, any one, some, or all of the release rates described herein in relation to another release rate can be characterized as also or alternatively being greater than the indicated release rate (e.g., a second release rate that is described as 1.25× that of a first release rate can alternatively be construed as disclosing an aspect characterized by exhibiting a release rate that is at least about 1.25× that of the first release rate).

In still other aspects, an indicated amount of nicorandil released in a period provides implicit support for a corresponding approximately minimum relative amount of drug released in a period, rather than a release rate (e.g., a disclosure of a first release rate and a second release rate that is 1.25× greater than the first release rate implicitly discloses aspects in which the amount of nicorandil compound released in the second period is at least about 1.25× the amount released in the first period).

### D. Delivery Facilitating Component (e.g., Capsule Vehicle)

In aspects the compositions disclosed herein comprise a delivery facilitating agent/vehicle, or which may also be referred to as a delivery facilitating agent/shell, such as a capsule. In another aspect the delivery facilitating agent contains both the first release tablets and the second release tablets in one single administration form (or contains most or generally all of the first and second release tablets/units). In an aspect, the delivery facilitating agent can be any suitable oral pharmaceutical form such as a hard capsule comprising two pieces that fit together, a soft capsule comprising a single piece, a cachet, and the like. In an aspect, a capsule can be made of any material and in any size suitable for pharmaceutical administration. In aspects, the capsule does not dissolve prior to reaching the stomach. In further aspects the capsule dissolves within about 3-6 minutes, such as within ˜3, ˜4 minutes, ˜5, or within about 6 minutes once it is exposed to the acidic environment of the stomach.

In aspects the capsule can be made of hard gelatin, HPMC, fish gelatin, starch, Pullulan, Polyvinyl acetate (PVA), soft gelatin. In further aspects, the capsule can be a size 000, 00, 0, 1, 2, 3, or 4. In aspects the size selected will be such that the quantity of first release tablets and the quantity of second release tablets can all fit within the capsule in any suitable position that permits for suitable capsule closure. In an aspect the capsule is an HPMC capsule. In another aspect, the capsule is a size 0 HPMC capsule. In yet another aspect, the capsule is a size 00 HPMC capsule. In aspects, a capsule can comprise a coloring such as red, orange, yellow, green, blue, purple, brown, grey, pink, white, beige, or variations or shades thereof.

In aspects, a delivery vehicle/capsule is mostly, generally only, or entirely composed of gelatin, pullulan, or HPMC. In aspects, capsules are composed of material having a moisture content of less than ˜6% (e.g., less than ˜5%, less than ˜4%, less than about ˜3%, less than ˜2%, or less than ˜1%. In aspects, the moisture content of the capsule material is stable at a moisture content (MC) of about 2% or about 3%. Relevant principles/materials adaptable to compositions in aspects of the invention are described in Yang et al., International Journal of Biological Macromolecules, Accepted May 15, 2020, DOI: https://doi.org/10.1016/j.ijbiomac.2020.05.110.

### E. Stability-Promoting Elements/Packaging

In aspects, the compositions comprise stability-promoting elements/packaging. In this respect the term “composition” means the combination of the formulation (which can comprise multiple dosage forms that collectively make up a first release component and a second release component) in combination with packaging element/components that detectably or significantly promote the stability of the formulation.

In aspects, a formulation comprises one or more ingredients that stabilize nicorandil from moisture-related destabilization. Examples of such ingredients include fumaric acid, dimethicone, simethicone, cyclodextrin derivatives, and combinations thereof. In aspects, compositions lack any or all of these or similar excipients.

In aspects, the composition is packaged with an effective amount of one or more anti-moisture agents, such as one or more wicking agents/desiccants (e.g., silica desiccant(s)). In aspects, packaging comprises unit does compositions comprising an approximately uniform amount of desiccant in association with each unit dose contained in the packaging. In aspects, the packaging comprises a blister pack packaging. In aspects, the blister pack packaging comprises at least one aluminum layer. In aspects, the blister packaging is an aluminum-aluminum blister pack. This type of packaging provides the most protection against moisture. In aspects, the blister pack packaging also or alternatively comprises a desiccant element, such as a desiccant-fitted aluminum foil component. Examples of such materials are sold by Amcor under the brand names Dessiflex Plus and Dessiflex Ultra. Alu Alu Blister Foil or CFF (Cold Forming Foil) is a film which is a multilayered structure consisting of Oriented Polyamide OPA (Nylon), Aluminum Foil and PVC film, which are stuck together using an adhesive, which can be a useful component of such packaging in aspects. An exemplary Alu Alu Foil specification is as follows: OPA (Nylon) film 25 Micron/Adhesive/Aluminum Foil 45 Micron/Adhesive/PVC 60 Micron OPA (Nylon) film 25 Micron/Adhesive/Aluminum Foil 50 Micron/Adhesive/PVC 60 Micron OPA (Nylon) film 25 Micron/Adhesive/Aluminum Foil 60 Micron/Adhesive/PVC 60 Micron. In an Alu Alu Blister pack, both the lidding and the base material consists of Aluminum Foil which makes it possible to protect the medicine from water vapor in the atmosphere, oxygen, and strong light. Such packaging components also are called Cold Form Blister because in the Alu Alu foil manufacturing process, the laminate is pressed in the mold without the help of any extra heat and is formed purely by Cold Form Technique. Forming pins are used to form the cavity in this film for the particular tablet or the capsule and then it is sealed with Aluminum Lidding material. Additional examples of similar/alternate materials include WisePac® blister desiccant foils produced by WiseSorbent Technology, Marlton, NJ 08053, USA. Examples of such materials are also described in, e.g., U.S. Pat. No. 9,834,356B2.

### F. Performance Characteristics of Compositions

In aspects, compositions and composition components (e.g., dosage forms) can be characterized on the basis of NCDC dissolution from the applicable composition/component.

i. Dissolution Profile of Capsules

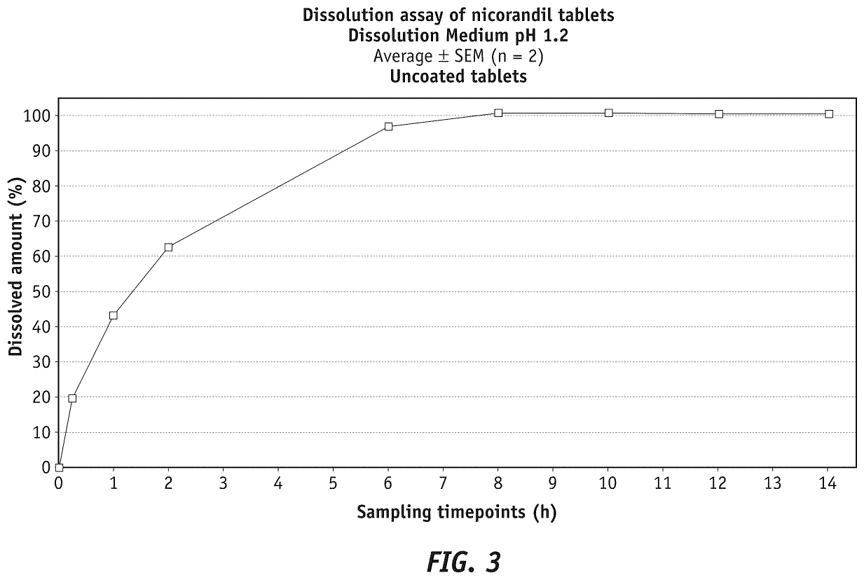

In aspects, contacting the uncoated, first release tablets with a dissolution medium having a pH of between about 1 and 1.5, such as 1.1, such as 1.2, such as 1.3, or such as 1.4, and contacting the coated, second release tablets with a dissolution medium having a pH of between about 6 and 7 (which mimics the later sections of the intestines), such as about 6.1, such as about 6.2, such as about 6.3, such as about 6.4, such as about 6.5, such as about 6.6, such as about 6.7, such as about 6.8, or such as about 6.9, both at a temperature of about room temperature, i.e. 37° C., and under moderate stirring for about 2 hours, the resulting profiles can then be combined to illustrate conceptually a combined release profile with two different pH levels over 24 hours, as shown in FIG. 3. In aspects, the dissolution profile is representative of the release profile of the capsule in vivo. In vivo, the profile includes two pulses, or peaks, the first being when half of the capsule (the uncoated, first release tablets) releases its contents in the first part of digestion (low pH between e.g. 1 and 1.5), while the other half (coated second release tablets) will release only at a pH higher than 5.5, in the later part of digestion. In aspects, this introduces a second pulse once the capsule reaches the intestine.

In aspects, the release profile of one or more compositions of the invention results in a first steady release period of about 5-6 hours after administration, and a second steady release of around 10-11 hours after administration as exemplified by the data in the Figures. As used herein, the term “pulse” can refer to the period wherein at least about 80%, 85%, or ≥˜90% of the composition, e.g., about 92.5% of the active ingredient, e.g., one or more nicorandil compound(s), has/have been released from the composition/dosage form.

In aspects, controlled release NCDC (e.g., nicorandil) capsules according to the invention (comprising first release dosage forms and second release dosage forms) release about 15-25%, e.g., ˜15-22%, ˜15-20%, ˜15-18%, ˜17-25%, ˜20-25%, ˜22-25%, e.g., ˜17-22%, of the therapeutically effective amount of nicorandil compound(s) contained therein at about one hour after administration. In aspects, controlled release NCDC (e.g., nicorandil) capsules can, in aspects, further release an additional detectable amount of the one or more nicorandil compounds such that about 20-40% of the therapeutically effective amount, e.g., ˜20-35%, ˜20-30%, ˜20-25%, e.g., ˜25-40%, ˜30-45%, ˜35-45%, or ˜40-45%, e.g., ˜25-25% of the therapeutically effective amount is released from the capsule at about two hours after administration. In aspects, controlled release NCDC (e.g., nicorandil) capsules can, in aspects, further release an additional detectable amount of the one or more nicorandil compounds such that about 35-50%, e.g., ˜35-45%, ˜35-40%, or ˜40-50%, e.g., ˜45-50%, as in, e.g., ˜40-45%, of the therapeutically effective amount is released from the capsule at about four hours after administration. In aspects, controlled release NCDC (e.g., nicorandil) capsules can, in aspects, further release an additional detectable amount of the one or more compounds such that about 45-60%, e.g., ˜45-55%, ˜50-55%, or ˜50-60%, ˜55-60%, or, e.g., ˜50-55%, of the therapeutically effective amount is released from the capsule at about six hours after administration. In aspects, controlled release NCDC (e.g., nicorandil) capsules can, in aspects, can further release an additional detectable amount of the one or more nicorandil compounds such that about 65-85%, e.g., ˜65-80%, ˜65-75%, ˜65-70%, or ˜70-85%, ˜75-85%, or ˜80-85%, such as ˜70-80%, of the therapeutically effective amount is released from the capsule at about eight hours after administration. In aspects, controlled release NCDC (e.g., nicorandil) capsules can further release an additional detectable amount of the one or more nicorandil compounds such that at least about 90%, e.g., ≥˜91%, ≥˜92%, ≥˜93%, ≥˜94%, ≥˜95%, ≥˜96%, ≥˜97%, ≥˜98%, or ≥˜99%, of the therapeutically effective amount is released about 16 hours after administration.

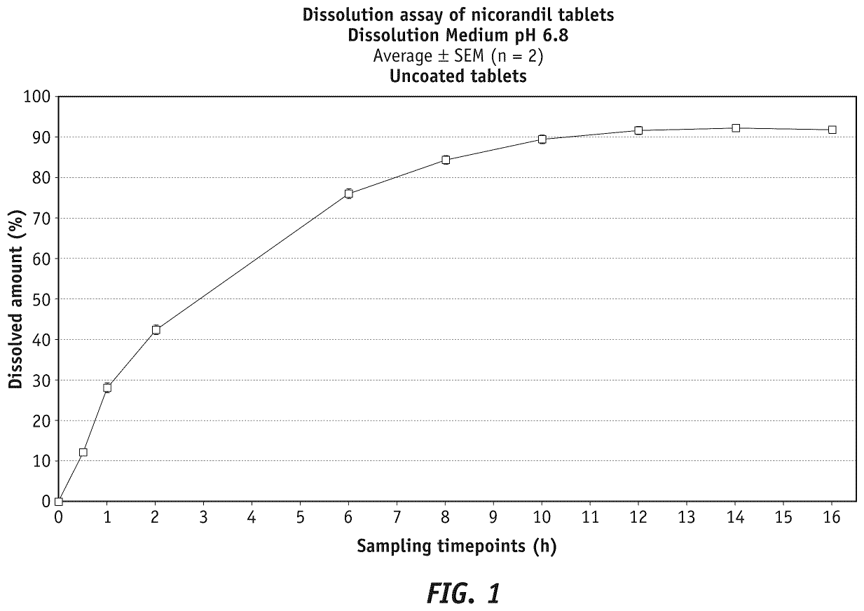

Release characteristics of exemplary compositions are provided in the Examples and reflected in the Figures submitted herewith. For example, in FIG. 3, the 50% point on the y axis is representative of 100% release of the of the first dose (20 mg) of the marketed Ikorel© formulation, which is administered twice daily, and only 50% (the first release tablets) of the total dose of the composition of the present invention (40 mg), which is taken once daily. FIG. 1 also demonstrates/exemplifies the difference in release profiles of the reference BiD Ikorel© and inventive compositions described herein. At each dose, the reference product releases nearly 90% of the active substance within ˜15 min in pH 1.2 and pH 6.8. The composition of the present invention releases nearly 90% of its first pulse (first 20 mg [one or more first release uncoated tablet component] of its total 40 mg) of the active substance in between about 5-6 hours in pH 1.2 (mimicking the acidic environment of the stomach) and its second pulse (second 20 mg [one or more second release coated tablets] of its total 40 mg) in about 10-11 hours in pH 6.8 (mimicking the later sections of the intestines).

ii. Purity Characteristics and Impurities

In aspects, compositions of the invention can be characterized on the basis of impurities in the composition, in the composition upon storage, released from the composition, or a combination of some or all thereof.

According to aspects, compositions of the invention are at least about 97% pure, at least about 98%, or at least about 99% pure in terms of nicorandil or a NCDC as compared to chemically similar impurities (e.g., imp D). In aspects, compositions maintain such levels of purity of a period of months of storage under the various stability test conditions described herein (e.g., after 3, 6, 9, 12, or 18 months at 5° C. or 25° C.).

According to aspects, compositions of the invention comprise an amount of impurities that is about the same as, significantly better than, or statistically similar to the exemplary impurity data provided in the Examples (in regard to most, generally all, or all measured impurities—e.g., imp D).

In aspects, a composition of the invention comprises total impurities in an amount less than about 5%, such as less than ˜4%, such as less than ˜3%, such as less than ˜2.5%, such as less than ˜2%, such as less than ˜1.5%, such as less than ˜1%, such as less than ˜0.5%, e.g., <˜0.4%, <˜0.3%, <˜0.2%, <˜0.1% or even less of total impurities. In aspects, the composition may comprise a largest unspecified impurity within the total impurities in an amount of less than ˜0.5%, such as less than ˜0.45%, such as less than ˜0.40%, such as less than ˜0.35%, such as less than ˜0.30%, such as less than ˜0.25%, such as less than ˜0.20%, such as less than ˜0.15%, such as less than ˜10%, or even less.

In aspects, a composition of the invention when initially contacted with a dissolution media of pH 1.2 for about 6 hours is associated with at least about 15%, such as at least about 20%, or at least about 25% less of one or more of imp A, imp B, imp C, imp D than Nikoran OD tablets. See, e.g., Example 18 herein.

iii. Bioavailability and Pharmacokinetic Properties

In aspects, compositions of the invention, administered once daily to a mammalian host, such as a human patient, are bioequivalent to twice-daily administration of a corresponding amount of NCDCs (e.g., nicorandil) in an immediate release (IR) formulation, such as the Ikorel formulation. In aspects, once daily administration of a composition of the invention is associated with a detectably or significantly longer period in which the plasma concentration of nicorandil is at least about 14.5 ng/mL as compared to twice-daily Ikorel administration, once daily Nikoran administration, or both.

### 2. Production of Compositions and Related Aspects

In aspects, the compositions disclosed herein may be produced using one or more manufacturing methods as known in the industry, such as wet granulation, dry granulation, and direct compression. In additional aspects, wet granulation may comprise one or more steps comprising weighing, mixing, adding a binder, wet screening, drying, screening/sizing granules, mixing with a disintegrant and/or lubricant, compressing into tablets. In other aspects, dry granulation may comprise weighing, mixing, compressing the mixture to form slugs, i.e., extra-large tablets, sieving and milling the slugs, mixing with a disintegrant and/or lubricant, compressing into tablets. In yet further aspects, direct compression may comprise weighing, milling, mixing, adding lubricants, mixing, and compression.

In an aspect, a number of formulations were prepared using a direct compression manufacturing process. During the process, first release and second release tablets were made by the following steps and placed in capsule as described below.
1. Weigh Nicorandil, MCC Prosolv HD90, STARTAB® starch, HPMC DC2, and Magnesium Stearate Ligamed MF-2-V
2. Mix components of step 1 using a mixer to achieve a dry blend
3. Compress the mixed blend into 5 mm tablet, target weight 100 mg, target hardness≥60N. Divide the yield into two equal parts, one part will be submitted for subsequent coating
4. Weigh out coating components and prepare the coating suspension
5. Mix the coating suspension to achieve a homogeneous mixture
6. Coat tablets in fluid bed dryer with the coating suspension
7. Dry the final batch in a fluid bed dryer
8. Fill HPMC capsule size 0 manually with 2 uncoated and 2 coated tablets and pack accordingly

In aspects, formulations prepared by this process can include the following types of compositions (with tablet formulations described in ranges of excipients that may be suitable and capsule formulations reflecting particular amounts of excipients within such ranges).

TABLE 1
Tablet Formulation 1

| | | | Exemplary Unit | |
|---|---|---|---|---|
| Ingredient | Unit weight (%) | | weight (mg) | |
| Nicorandil | 7.5-12.5 | (e.g., 10) | 7.5-12.5 | (e.g., 10) |
| MCC Prosolv HD90 | 30-40 | (e.g., 34.5) | 30-40 | (e.g., 34.5) |

| | | |
|---|---|---|
| STARTAB ® starch | 0-30, e.g., 20 | 0-30, e.g., 20 |
| HPMC DC2 | 20-50, e.g., 35 | 320-50, e.g., 35 |
| Magnesium stearate | 0.1-1, e.g., 0.5 | 0.1-1, e.g., 0.5 |
| Ligamed MF-2-V | | |
| Total Core Tablet | 100.0 | 100.0 |
| Coating: | | |
| Eudragit L30 D-55 | | 5-7.5, e.g., 6.66 |
| Talc | | 1.5-3.5, e.g., 2.66 |
| Triethylcitrate | | 0.1-1, e.g., 0.66 |
| Demiwater | | Qs |

TABLE 1a
Total Amount of Formulation 1 in Exemplary Capsule

| Ingredient | Unit weight (mg) |
|---|---|
| Nicorandil | 40.0 |
| MCC Prosolv HD90 | 138 |
| STARTAB ® starch | 80.0 |
| HPMC DC2 | 140 |
| Magnesium stearate Ligamed MF-2-V | 2 |
| Coating: | |
| Eudragit L30 D-55 | 13.32 |
| Talc | 5.32 |
| Triethylcitrate | 1.32 |
| Demiwater | Qs |
| Total Capsule | 420.0 |

TABLE 2
Tablet Formulation 2

| Ingredient | Unit weight (%) | Unit weight (mg) |
|---|---|---|
| Nicorandil | 7.5-12.5 (e.g., 10) | 7.5-12.5 (e.g., 10) |
| MCC Prosolv HD90 | 35-60, 40-50, e.g., 44.5 | 35-60, 40-50, e.g., 44.5 |
| STARTAB ® | 0-15, e.g., 3-15, | 0-15, e.g., 3-15, |
| starch | 5-15, or 10 | 5-15, or 10 |
| HPMC DC2 | 20-50, 30-40, or 35 | 20-50, 30-40, or 35 |
| Magnesium stearate | 0.1-1, 0.2-0.8, or 0.5 | 0.1-1, 0.2-0.8, or 0.5 |
| Ligamed MF-2-V | | |
| Total Core Tablet | 100.0 | 100.0 |
| Coating: | | |
| Eudragit L30 D-55 | | 5-7.5, e.g., 6.66 |
| Talc | | 1.5-3.5, e.g., 2.66 |
| Triethylcitrate | | 0.1-1, e.g., 0.66 |
| Demiwater | | Qs |

TABLE 2a
Total Amount of Formulation 2 in Exemplary Capsule

| Ingredient | Unit weight (mg) |
|---|---|
| Nicorandil | 40.0 |
| MCC Prosolv HD90 | 178 |
| STARTAB ® starch | 40.0 |
| HPMC DC2 | 140 |
| Magnesium stearate Ligamed MF-2-V | 2 |
| Coating: | |
| Eudragit L30 D-55 | 13.32 |
| Talc | 5.32 |
| Triethylcitrate | 1.32 |
| Demiwater | Qs |
| Total Capsule | 420.0 |

TABLE 3
Tablet Formulation 3

| Ingredient | Unit weight (%) | Unit weight (mg) |
|---|---|---|
| Nicorandil | 7.5-12.5 (e.g., 10) | 7.5-12.5 (e.g., 10) |
| MCC Prosolv HD90 | 35-65, e.g., 50-60, | 35-65, e.g., 50-60, |
| | e.g., 54.5 | e.g., 54.5 |
| STARTAB ® starch | 0 | 0 |
| HPMC DC2 | 20-50, 30-40, or 35 | 20-50, 30-40, or 35 |
| Magnesium stearate | 0.1-1, 0.2-0.8, or 0.5 | 0.1-1, 0.2-0.8, or 0.5 |
| Ligamed MF-2-V | | |
| Total Core Tablet | 100.0 | 100.0 |
| Coating: | | |
| Eudragit L30 D-55 | | 5-7.5, e.g., 6.66 |
| Talc | | 1.5-3.5, e.g., 2.66 |
| Triethylcitrate | | 0.1-1, e.g., 0.66 |
| Demiwater | | Qs |

TABLE 3a
Total Amount of Formulation 3 in Exemplary Capsule

| Ingredient | Unit weight (mg) |
|---|---|
| Nicorandil | 40.0 |
| MCC Prosolv HD90 | 218 |
| STARTAR ® starch | 0 |
| HPMC DC2 | 140 |
| Magnesium stearate Ligamed MF-2-V | 2 |
| Coating: | |
| Eudragit L30 D-55 | 13.32 |
| Talc | 5.32 |
| Triethylcitrate | 1.32 |
| Demiwater | Qs |
| Total Capsule | 420.0 |

TABLE 4
Tablet Formulation 4

| Ingredient | Unit weight (%) | Unit weight (mg) |
|---|---|---|
| Nicorandil | 7.5-12.5 (e.g., 10) | 10.0 |
| MCC Prosolv HD90 | 20-40, 25-35, e.g., 29.5 | 20-40, 25-35, e.g., 29.5 |
| STARTAB ® | 0-30, 5-30, 5-25, 10-25, | 0-30, 5-30, 5-25, 10-25, |
| starch | 15-25, 20 | 15-25, 20 |
| HPMC DC2 | 25-55, 30-50, 40 | 25-55, 30-50, 40 |
| Magnesium stearate | 0.1-1, 0.2-0.8, or 0.5 | 0.1-1, 0.2-0.8, or 0.5 |
| Ligamed MF-2-V | | |
| Total Core Tablet | 100.0 | 100.0 |
| Coating: | | |
| Eudragit L30 D-55 | | 5-7.5, e.g., 6.66 |
| Talc | | 1.5-3.5, e.g., 2.66 |
| Triethylcitrate | | 0.1-1, e.g., 0.66 |
| Demiwater | | Qs |

TABLE 4a
Total Amount of Formulation 4 in Exemplary Capsule

| Ingredient | Unit weight (mg) |
|---|---|
| Nicorandil | 40.0 |
| MCC Prosolv HD90 | 118 |
| STARTAB ® starch | 80.0 |
| HPMC DC2 | 160 |
| Magnesium stearate Ligamed MF-2-V | 2 |
| Coating: | |
| Eudragit L30 D-55 | 13.32 |
| Talc | 5.32 |
| Triethylcitrate | 1.32 |
| Demiwater | Qs |
| Total Capsule | 420.0 |

TABLE 5
Tablet Formulation 5

| Ingredient | Unit weight (%) | Unit weight (mg) |
|---|---|---|
| Nicorandil | 7.5-12.5 (e.g., 10) | 7.5-12.5 (e.g., 10) |
| MCC Prosolv HD90 | 25-55, 30-50, 39.5 | 25-55, 30-50, 39.5 |
| STARTAB ® starch | 0-20, 5-20, 5-15, 10 | −20, 5-20, 5-15, 10 |
| HPMC DC2 | 30-50, 35-45, 40 | 30-50, 35-45, 40 |
| Magnesium stearate | 0.1-1, 0.2-0.8, or 0.5 | 0.1-1, 0.2-0.8, or 0.5 |
| Ligamed MF-2-V | | |
| Total Core Tablet | 100.0 | 100.0 |
| Coating: | | |
| Eudragit L30 D-55 | | 5-7.5, e.g., 6.66 |
| Talc | | 1.5-3.5, e.g., 2.66 |
| Triethylcitrate | | 0.1-1, e.g., 0.66 |
| Demiwater | | Qs |

TABLE 5a
Total Amount of Formulation 5 in Exemplary Capsule

| Ingredient | Unit weight (mg) |
|---|---|
| Nicorandil | 40.0 |
| MCC Prosolv HD90 | 158 |
| STARTAR ® starch | 40.0 |
| HPMC DC2 | 160 |
| Magnesium stearate Ligamed MF-2-V | 2 |
| Coating: | |
| Eudragit L30 D-55 | 13.32 |
| Talc | 5.32 |
| Triethylcitrate | 1.32 |
| Demiwater | Qs |
| Total Capsule | 420.0 |

TABLE 6
Tablet Formulation 6

| Ingredient | Unit weight (%) | Unit weight (mg) |
|---|---|---|
| Nicorandil | 7.5-12.5 (e.g., 10) | 7.5-12.5 (e.g., 10) |
| MCC Prosolv HD90 | 25-55, 30-50, 39.5 | 25-55, 30-50, 39.5 |
| STARTAB ® starch | 0 | 0 |
| HPMC DC2 | 30-50, 35-45, 40 | 30-50, 35-45, 40 |
| Magnesium stearate | 0.1-1, 0.2-0.8, or 0.5 | 0.1-1, 0.2-0.8, or 0.5 |
| Ligamed MF-2-V | | |
| Total Core Tablet | 100.0 | 100.0 |
| Coating: | | |
| Eudragit L30 D-55 | | 5-7.5, e.g., 6.66 |
| Talc | | 1.5-3.5, e.g., 2.66 |
| Triethylcitrate | | 0.1-1, e.g., 0.66 |
| Demiwater | | Qs |

TABLE 6a
Total Amount of Formulation 6 in Exemplary Capsule

| Ingredient | Unit weight (mg) |
|---|---|
| Nicorandil | 40.0 |
| MCC Prosolv HD90 | 198 |
| STARTAB ® starch | 0 |
| HPMC DC2 | 160 |
| Magnesium stearate Ligamed MF-2-V | 2 |
| Coating: | |
| Eudragit L30 D-55 | 13.32 |
| Talc | 5.32 |
| Triethylcitrate | 1.32 |
| Demiwater | Qs |
| Total Capsule | 420.0 |

TABLE 7
Tablet formulation 7

| Ingredient | Unit weight (%) | Unit weight (mg) |
|---|---|---|
| Nicorandil | 7.5-12.5 (e.g., 10) | 7.5-12.5 (e.g., 10) |
| MCC Prosolv HD90 | 25-45, 30-40, 34.5 | 25-45, 30-40, 34.5 |
| STARTAB ® starch | 0-30, 5-30, 5-25, | 0-30, 5-30, 5-25, |
| | 10-25, 15-25, 20 | 10-25, 15-25, 20 |
| HPMC K100M CR | 30-50, 35-45, 40 | 30-50, 35-45, 40 |
| Magnesium stearate | 0.1-1, 0.2-0.8, or 0.5 | 0.1-1, 0.2-0.8, or 0.5 |
| Ligamed MF-2-V | | |
| Total Core Tablet | 100.0 | 100.0 |
| Coating: | | |
| Eudragit L 30 D-55 | | 5-7.5, e.g., 6.66 |
| Plasacryl HTP20 | | 0.5-1.5, 0.75-1.25, 1-1.1 |
| Colorant | | 0.005-0.015, e.g., 0.01 |
| Demiwater | | Qs |

TABLE 7a
Total Amount of Formulation 7 in Exemplary Capsule

| Ingredient | Unit weight (mg) |
|---|---|
| Nicorandil | 40.0 |
| MCC Prosolv HD90 | 138.0 |
| STARTAR ® starch | 80.0 |
| HPMC K100M CR | 140.0 |
| Magnesium stearate Ligamed MF-2-V | 2.0 |
| Coating: | |
| Eudragit L 30 D-55 | 13.32 |
| Plasacryl HTP20 | 4.4 |
| Colorant | 0.04 |
| Demiwater | Qs |
| Total Capsule | 420.00 |

### 3. Pharmaceutical/Therapeutic Uses

The compositions described herein can be used to induce/promote or cause any change in physiological condition that can be obtained through the delivery of effective amounts of nicorandil to a mammalian subject, such as a human patient, e.g., induction of nicorandil vasodilation or other physiological conditions described elsewhere in this disclosure. In aspects, the compositions of the present invention can be administered to a patient in need thereof for the treatment or reduction of symptoms of chronic stable angina pectoris. In an aspect the symptoms can comprise chest pain and/or discomfort in the local area of the chest. In aspects, administering an effective amount of a composition of the invention, once daily, can significantly or detectably shorten the QT segment. In aspects, administering an effective amount of a composition of the invention, once daily, can significantly or detectably treat congestive heart failure (CHF).

In another aspect, the compositions can be administered to a patient in need thereof for the treatment or reduction of symptoms of microvascular angina. In yet another aspect, the compositions of the present invention can be administered to a patient in need thereof for the reduction in risk of cardiovascular mortality. In a further aspect, the compositions of the present invention can be administered to a patient in need thereof for the reduction in risk of non-fatal myocardial infarction. In another aspect, the compositions of the present invention can be administered to a patient in need thereof for the reduction in risk of non-fatal stroke.

In another aspect, the compositions of the present invention can be administered to a patient in need thereof for the reduction risk of transient ischemic attacks requiring hospital admission. In a further aspect, the compositions of the present invention can be administered to a patient in need thereof for the reduction in risk of unplanned hospital admission for cardiac chest pain. In aspects, the compositions of the present invention are delivered as a therapeutically effective once-daily regimen for the treatment of any one or more of the above-described diseases/conditions.

In aspects, methods comprise delivery of an effective amount of compositions of the invention, to detectably or significantly improve arterial vasodilation, promote venous relaxation, promote cardioprotective effects, or a combination thereof. In aspects, administering an effective amount of a composition (e.g., once daily, as can be applied to any of the method/use aspects described herein) detectably or significantly improves angina symptoms both in intensity and frequency. In aspects, administering an effective amount of a composition results in a detectable or significant plaque-stabilizing effect in patients, such as CSAP patients. In aspects, In aspects, administering an effective amount of a composition detectably or significantly improves endothelial function, e.g., in patients with underlying CAD. In aspects, administering an effective amount of a composition is effective to treat unspecified chronic stable angina. In aspects, administering an effective amount of a composition is effective at treating angina symptoms in MVA patients. Additional methods and impacts thereof (therapeutic/physiological targets or endpoints) are described in the following sections. As noted, readers will understand that any statement of efficacy herein can be, in aspects, determined in a suitably powered population of subjects/patients, e.g., in a well-controlled adequate clinical study.

Various applications/uses and methods are also provided in the Exemplary Aspects that can be combined with the teachings in this and related sections of this disclosure (e.g., one or more of aspects 91-99).

### Administration Conditions and Physiological/Therapeutic Effects

In aspects, administration of a composition results in about 60% of the total dose contained in the first dosage form being dissolved after 2 hours from administration and over 80% of the total dose contained in the tablet is dissolved after about 4 hours. At this time, and given the average residence time of the stomach content of 4.5 hours, the amount of the active available for absorption and activity past the stomach is no more than 20% of the total dose contained in the tablet. In aspects, at 6 hours, the time at which most of the stomach content would have emptied, 95% of the total dose contained in the tablet is dissolved, making approximately 5% of the total dose contained in the first release component is available for absorption and activity 6 hours after administration. However, at such 4- and 6-hour periods less than 20% (e.g., less than 10%) of the nicorandil contained in the second dosage form is released. Accordingly, at least about 40%, such as about 45%, about 50%, or about 55% of the total amount of NCDCs in the composition (e.g., capsule comprising first release dosage forms and second release dosage forms) is available as the composition reaches the intestine. As reflected in the Examples and the Exemplary Aspects, compositions according to such aspects exhibit significantly longer/prolonged nicorandil release as compared to Nikoran (e.g., at 6 hours post contact with a pH 1.2 media there is less than 10% of the 10 mg Nikoran tablet available (approximately <1 mg)).

According to aspects, once daily administration of a composition of the invention results in an increase in average exercise duration by about 13% to 35% from baseline at 6, 8, 12, 14, or 18 hours after administration at 6, 8, 12, 14, or 18 hours after administration. Also or alternatively administration of an effective once-daily amount of compositions results in delaying the time to onset of angina by about 20% to 40% from baseline at 6, 8, 12, 14, or 18 hours after administration. Still further also or alternatively, once daily administration of a composition of the invention results in delaying the time to ≥1 mm ST-segment depression by about 20% to 50% from baseline at 6, 8, 12, 14, or 18 hours after administration.

In aspects, mean reduction in blood pressure (especially DBP) up to 20 mmHg is preserved after ˜2, ˜6, ˜12, or (and/or), e.g., ˜18 hours after single administration, e.g., as a result of releasing the second dose (coated tablets releasing the active past the stomach) in patients with angina.

In aspects, administration of a composition of the invention results in a plasma concentration of ≥12 ng/mL, such as ≥14 or ≥14.5 ng/mL at 6, 8, 10, or 12 hours post administration. In aspects, once-daily administration of compositions exhibits at least non-inferiority to twice-daily administration of Ikorel (IR) tablets or a corresponding NCDC composition comprising the Ikorel tablet formulation.

In aspects, administration of the composition retains a 10-15% (e.g., ˜10-14%, ˜10-13%, ˜10-12%, or ˜10-11%, such as, e.g., ˜11-15%, ˜12-15%, ˜13-15%, or ˜14-15%) increase in mean luminal diameter of the epicardial coronary arteries for a significantly longer period than BID Ikorel administration or QD Nikoran administration. In aspects, administration of the composition retains a 10-15% increase in mean luminal diameter of the epicardial coronary arteries at 8, 10, 12, 14, or 16 hours post administration.

In aspects, the persistence of nicorandil clinical effects upon once-daily administration of the composition lasts for at least ˜12 hours, e.g., at least about 13, 14, 15, 16, 17, 18, 19, 20, 21, 22, 23, or, e.g., at least about 24 hours in a significant number of patients.

In aspects, the compositions are administered to prevent ischemic reperfusion injury, improve myocardial ischemia, modulate antiplatelet effects, improve non-sustained ventricular tachycardia, improve microvascular perfusion, relieve microvascular spasm and reduce platelet aggregation, reduce/prevent microvascular dysfunction during percutaneous intervention, improve myocardial salvage, dilate one or both coronary microvessels and peripheral resistance arteries, inhibiting the apoptosis and inflammatory reaction of myocardial cells, reduce arrhythmic burden, inhibiting the apoptosis and inflammatory reaction of myocardial cells, improve stabilization of atherosclerotic plaques, suppression of endoplasmic reticulum stress-induced apoptotic cell death through the PI3K/Akt pathway. or result in a combination thereof, e.g., in myocardial infarction patients or in patients with angina, e.g., unstable angina. In aspects, administration of the compositions significantly reduces the rate of mortality in a population of patients treated with the compositions over a period of time (e.g., ≥6 months, ≥12 months, ≥18 months, ≥2 years, or ≥2.5 years). In aspects, compositions are administered to provide prophylaxis of effort-induced angina, angina due to coronary spasm, ‘mixed’ angina, microvascular dysfunction, or a combination thereof. In aspects, administration of the compositions results in a detectable or significant reduction in frequency of angina episodes, DOS improvement of exercise tolerance following treatment, DOS increase time to angina during exercise, or a combination thereof. In aspects, compliance of treatment is DOS improved in a population as a result of performing the method. E.g., in aspects, ≥80%, 85%, or >90% of patients in a monitored population continue using the composition for a period of more than 1 year. In aspects, methods are practice without co-administration of PDE-5 inhibitors. Corticosteroids, sulfonylureas, including glibenclamide and glimepiride, calcium channel blockers, or a combination thereof. In aspects, methods improve the grading of a significant number of angina patients during the course of treatment (e.g., using the Canadian Cardiovascular Society Grading of Angina Pectoris Scale).

### Indications/Diseases Treated and Related Conditions of Use

In aspects, compositions of the invention are administered, e.g., once daily, to treat adult patients with chronic stable anginal pectoris, e.g., patients who are inadequately controlled on or have a contraindication or intolerance to frequently used anti-anginal therapies (such as beta-blockers, long-acting nitrates, and/or calcium antagonists).

In aspects, methods are performed to treat patients having or diagnosed with microvascular angina (MVA) (e.g., in patients with normal coronary angiography examination results, where after excluding other organic heart diseases, the clinical manifestations are chest pain, myocardial ischemia, and microcirculation disorders).

In aspects, methods of the invention are performed to treat chronic-stable angina pectoris (CSAP), coronary microvascular disease (CMD), or both. In aspects, methods of the invention are performed to treat angina, exertional dyspnea, coronary artery disease (CAD), or risk of heart failure (or CHF).

In another aspect, the compositions of the present invention can be administered once daily to a patient in need thereof for the symptomatic treatment (reductions of anginal symptoms) of chronic stable angina pectoris. In aspects, methods result in reductions of anginal symptoms of chronic stable angina pectoris.

In another aspect, the compositions can be administered to a patient once daily in need thereof for the treatment or reduction of symptoms of symptoms of microvascular angina; i.e. microvascular aspects of chronic stable angina. In aspects, stable plasma levels of nicorandil QD over 24 hours pursuant to the present invention provide improvements to microvascular function inherently superior to the current immediate-release version of the compound.

In yet another aspect, the compositions of the present invention can be administered to a patient in need thereof for the reduction in risk of cardiovascular mortality, reduction in risk for acute coronary syndromes, or both.

In a further aspect, the compositions of the present invention can be administered to a patient in need thereof for the reduction in risk of non-fatal myocardial infarction.

In another aspect, the compositions of the present invention can be administered to a patient in need thereof for the reduction in risk of non-fatal stroke (i.e., ischemic stroke), e.g., in aspects with the understanding that continuous stable plasma levels of a QD formulation of nicorandil with stable continuous plasma drug levels over 24 hours pursuant to the present invention provide improvements to ischemic stroke prevention inherently superior to the current immediate-release version of the compound.

In another aspect, the compositions of the present invention can be administered to a patient in need thereof for the reduction risk of transient ischemic attacks requiring hospital admission.

In a further aspect, the compositions of the present invention can be administered to a patient in need thereof for the reduction in risk of unplanned hospital admission for angina pectoris symptoms (i.e., cardiac chest pain).

In a further aspect, the compositions of the present invention can be administered to a patient in need thereof for avoiding and thus the reduction in cost of unplanned hospital admission and procedures for cardiac chest pain.

In aspects, performance of methods of the invention is at effective for improving symptoms, outcomes, or conditions in patients undergoing elective PCI with coronary artery disease in terms of reducing the incidence of adverse cardiovascular events as well as improving heart function. In aspects, methods of the invention are performed as an adjunctive therapy accompanying PCI procedures.

In aspects, methods of the invention result in detectably or significantly improved safety, tolerability, or both, as compared to BID IR nicorandil formulations (e.g., in terms of lower nicorandil-associated adverse events (AEs), such as headache, GI side effects, or both).

In aspects, methods of the invention are proven to reduce the risk of underlying atherosclerotic cardiovascular disease in a multicenter, randomized double-blind, placebo-controlled outcomes study.

In aspects, methods of the invention result in a detectable or significant improvement in myocardial perfusion during rest, during exercise, or both.

In aspects, methods of the invention result in detectable or significant myocardial protective benefits (i.e., cardioprotection). reduce transient myocardial ischemia and tachyarrhythmias among, or both, in a manner that is at least non-inferior/therapeutically equivalent or superior to such effects associated with an IR nicorandil formulation, such as Ikorel.

In aspects, methods of the invention result in detectable or significant improvements in endothelial function in subjects.

In aspects, patients treated by methods are elderly individuals (e.g., aged≥65, ≥70, ≥75, or ≥80), are individuals with renal impairment, or both.

In aspects, patients have advanced coronary artery disease, refractory angina pectoris, or both, and the method is performed to improve one or more conditions of the patient (e.g., DOS reduce cardiovascular events and DOS improve functional angina class). In aspects, methods of the invention reduce the frequency and duration of refractory angina pectoris.

In aspects, patients have congestive heart failure (CHF), and methods of the invention DOS improve one or more conditions therein. E.g., in aspects methods of the invention results in a DOS mean decrease in arterial and pulmonary wedge pressure, a DOS increase in cardiac index, or both, in patients, such as CHF patients. In aspects, performing methods of the invention improves hemodynamic abnormalities of heart failure without concomitant tachycardia or increases in plasma catecholamines. In aspects, methods of the invention DOS reduce mortality in patients with ischemic heart failure.

In aspects, performing methods of the invention in patients leads to an abbreviation (shortening) of long QT syndrome, a reduction in torsades de pointes/arrythmia or both. In aspects, methods are associated with DOS long-term control and prevention of QT-prolongation in the form of steady reduction of the QT phase, e.g., as compared to BID IR nicorandil formulation administration methods.

According to aspects, methods of the invention are associated with a lower risk of developing CIN Contrast-induced nephropathy (CIN), e.g., in patients undergoing CAG/PCI with contrast-induced acute kidney injury (CI-AKI) percutaneous coronary intervention (PCI), or both. In aspects, methods further comprise administering an effective amount of one or more RAAS inhibitors—such as an ACE-inhibitor or angiotensin receptor blocker, with or in association with the administration of the once-daily two-pulse release NCDC formulation/composition.

In aspects, methods of the invention provide a steady activation of the mitochondrial KATP channels stimulating chronically the activity of MnSOD, providing DOS protection against oxidative stress and damage.

In aspects, methods of the invention are performed to treat or modulate one or more conditions in chemotherapy patients. In exemplary aspects, methods of the invention DOS protect the myocardium of chemotherapy patients using anthracycline-containing chemotherapy regimens from anthracycline-induced cardiotoxicity. In aspects, performing methods of the invention DOS improves heart function of chemotherapy patients, allows chemotherapy patients to DOS continue to maintain potentially life-saving chemotherapy use, or both. In aspects, methods of the invention are associated with DOS mitoKATP opening without the participation of the NO/cGMP dependent pathway. In aspects, methods of the invention DOS prevent the development of doxorubicin-induced heart failure without affecting the antitumor activity of the anthracycline medication. In aspects, methods of the invention result in maintaining the mitochondrial Katp channels open for a DOS longer time. In aspects, methods of the invention DOS condition cardiomyocytes to deal with periods of ischemia and protect the heart from oxidative damage. In aspects, methods of the invention result in DOS prolonged activity of the mitochondrial KATP channels in cardiomyocytes and provide sustained cardioprotection, reduced myocardial workload (induced pharmaceutically or through reduced physical activity), improved heart regeneration, or a combination thereof.

### Co-Administration, Combination Products, and Combined Therapies and Exclusions

In aspects, methods of the invention are performed in association with other therapies or administration of other APIs/drugs. In aspects, compositions can comprise other APIs. In aspects, methods are directed to treating CSAP and associated drugs administered to patients include beta-blockers, calcium channel blockers (CCBs), long-acting nitrates or ranolazine, or other inhibitor of the late sodium current in myocytes, or combination thereof. In aspects, one or more of such drugs are excluded from being administered as part of methods of the invention or are lacking from being present in compositions of the invention. In aspects, methods or compositions do not comprise inclusion of nitrate/nitrate ester vasodilators.

### 4. Lack of Particular Constituents or Characteristics

In certain aspects, composition(s) herein can be characterized by the lack of one or more constituent(s) or characteristic(s). Disclosure of any one or more constituent(s) or characteristic(s) of composition(s)/formulation(s) provided herein should be interpreted as further disclosing the express absence of such constituent(s) or characteristic(s) of composition(s)/formulation(s). Further, particular embodiments of composition(s)/formulation(s) lacking constituent(s), characteristic(s), or both are provided here.

According to aspects, composition(s) provided herein are not characterizable as comprising an inner immediate release compartment comprising one or more NCDC(s) surrounded by a release suppression compartment which does not comprise one or more NCDC(s) wherein the inner immediate release compartment is further surrounded by an outer immediate compartment comprising additional nicorandil NCDC(s).

According to certain aspects, composition(s) provided herein do not comprise one or more of (e.g., one or a combination of) fumaric acid, oxalic acid, stearyl fumaric acid sodium, an anhydrous ethanol, pyrrolidone, polyvinyl alcohol, polyvinyl acetate phthalate, methacrylic acid copolymer, hydroxypropyl methylcellulose, hydroxypropyl methylcellulose phthalate, hydroxypropyl methylcellulose succinate, hydroxymethylcellulose, hydroxyethylcellulose, methylcellulose, carboxymethylcellulose sodium, hydroxypropylmethylcellulose acetate succinate, shellac, cellulose acetate, cellulose acetate phthalate, cellulose propionate phthalate, a poly(methacrylate-methyl methacrylate) copolymer, a poly (methacrylate-ethyl acrylate) copolymer (such as one or more of a poly (ethyl acrylate-methyl methacrylate) copolymer or poly (ethyl acrylate-methyl methacrylate-trimethylaminoethyl methacrylate)), polyvinyl acetate, polymethacrylate copolymer, ethyl cellulose, mannitol, maltose, xylitol, maltitol, sorbitol, gelatinized starch, sodium starch glycolate, croscarmellose sodium, crosslinked polyvinylpyrrolidone, ion exchange resin, polyethylene oxide(s), polyethylene glycol(s), chitosan, gum(s), starch derivative(s), polyurethane(s), galactomannan(s), polysaccharide(s), polyalcohol(s), acrylic acid or acrylamide derivative(s), glycerol palmitostearate, beeswax, glycowax, carnaubawax, hydrogenated vegetable oil, glycerol monostearate, stearylalcholo, glyceryl behenate, polyanhydride(s), pectin, zein, modified zein, casein, gelatin, gluten, serum albumin, collagen, dextran(s), tamarind seed polysaccharide, gellan, carrageenan, xanthan gum, gum Arabic, hyaluronic acid, polyhyaluronic acid, alginic acid, sodium alginate, polyamide(s), polycarbonate(s), polyalkylene(s), polyalkylene glycol(s), polyalkylene oxide(s), polyalkylene terephthalate(s), polyvinyl alcohol(s), polyvinyl ether(s), polyvinyl ester(s), polyvinyl halide(s), polyvinylpyrrolidone, polyglycolide(s), polysiloxane(s), polyurethane(s), polystyrene, polymer(s) of acrylic and methacrylic ester(s), polylactide(s), poly(butyric acid), poly(valeric acid), poly(lactide-co-glycolide), polyanhydride(s), polyorthoster(s), poly(fumaric acid), poly(maleic acid), corn starch, lactose, croscarmellose sodium, crospovidone, glyceryl monostearate, colloidal silica, triethyl citrate, isopropyl alcohol, methanol, talc, or, e.g., castor oil.

In aspects, composition(s) comprise nicorandil compound(s) as the only API(s) present in the composition(s). That is, for example, in aspects, composition(s) do not comprise a beta 1-selective adrenoceptor blocking agent, e.g., metoprolol succinate.

In aspects, less than 90% of NCDC(s) present in composition(s) provided herein at the time of their administration have been released at about 4 hours, ˜5 hours, ˜6 hours, ˜7 hours, ˜8 hours, ˜9 hours, or, e.g., ˜10 hours post-administration.

In aspects, any element(s) of any of the references in the art described in the Background section of this disclosure can be excluded from any non-conflicting aspect of this disclosure.

### REPRESENTATIVE EXPERIMENTS/EMBODIMENTS (“EXAMPLES”)

The following detailed exemplary expository descriptions or experiments involving embodiments, applications, or related principles, of or otherwise related to the invention (“Examples”) are provided to assist readers in further understanding aspects of the invention or principles related to the invention or practice of aspects of the invention.

Any particular materials, methods, steps, and conditions employed/described in the following Examples, and any results thereof, are merely intended to further illustrate aspects of the invention. These Examples reflect exemplary embodiments of the invention, and the specific methods, findings, principles of such Examples, and the general implications thereof, can be combined with any other part of this disclosure. However, readers should understand that the invention is not limited by these Examples or any part thereof.

### Example 1

The following method was determined to be effective for production of exemplary compositions comprising first release profile tablets and second release profile tablets of the invention contained in a delivery facilitating capsule. This general method was employed in the production of such tablets/dosage forms and compositions as described in the remainder of these Examples.

Nicorandil, MCC Prosolv HD90, STARTAB starch, HPMC DC2, and Magnesium Stearate Ligamed MF-2-V were obtained in desired amounts and mixed using a standard mixing device (Turbula mixer at speed of 22 rpm) to arrive at a first dry blend of ingredients. Magnesium stearate was screened over a 500 μm sieve, weighed and add to the dry mix, which was then mixed for an additional 5 minutes at 22 rpm.

The dry mix was then compressed by direct compression into 5-6 mm round biconvex tablets, target weight 100 mg, and target hardness of at least 60N, optionally at least 80 N, optionally at least 100 N, and optionally at least 110 N or at least ˜120N.

The yield was divided into two equal parts, with one part (here one half of the 4 tablets of each capsule, i.e., 2 tablets) being subjected to coating, the others remaining in an uncoated form.

A coating suspension was mixed by first suspending talc thoroughly in 90% of demiwater for 15 minutes and preparing a second coating ingredient suspension formed by mixing Eudragit and triethylcitrate gently for 30 minutes. Suspension 1 was then added to suspension 2 and the mixture was subject to mixing for at least 15 minutes and screened over a 0.25 mm sieve.

Coated tablets were coated in a UniGlatt device with the coating suspension. The coating parameters were: Tproduct 30±3° C., Atom. air 1.5 bar, nozzle: 1.2 mm. Suggested ranges are: Tinlet=40-60° C., Vinlet=55-60 m3/hr, and spray rate=5-10 gram/min.

The final batch was dried in the UniGlatt until the LOD of the tablets was between 3 and 4% (Mettler HG53, switch-off criterion 4, T=105° C., 2 gram).

HPMC capsules with capsule size 0 (or OEL or 00) were filled manually with 2 uncoated and 2 coated tablets and packed accordingly.

### Example 2

An experiment was performed to analyze the dissolution profile of exemplary uncoated, first (general) release tablets according to an aspect of the invention at pH 6.8.

Individual tablets used in the experiment were manufactured according to the process described in Example 1 (a very similar process applied to similar, but different ingredients, also is described below in connection with FIG. 21).

The formulation of tablets and capsules used in this experiment and several other experiments described herein is shown in Table 8 (represented by the formulation without the coating ingredients).

TABLE 8
Nicorandil Tablet Composition

| Component | Unit weight (range %) | Unit weight (mg) |
|---|---|---|
| Nicorandil | 10 | 10 |
| MCC Prosolv HD90 | 34-54.5 | 34-54.5 |
| Starch Starlab | 0-20 | 0-20 |
| HPMC DC2 | 27.5-42 | 27.5-42 |
| Magnesium stearate | 0.5-1 | 0.5-1.0 |
| Ligamed MF-2-V | | |
| Total core tablet | | 100 |
| Coating: | | |
| Eudragit L30 D-55 | | 6.66-10 |
| Talc | | 0-2.66 |
| Triethylcitrate | | 0.66 |
| Demiwater | qs | |

Two first release (general/uncoated) tablets were collected for dissolution testing using a USP apparatus type II system (Agilent) at 50 rpm. Dissolution testing of the capsules was performed using 900 mL of a dissolution medium having a pH of 6.8 and maintained at 37° C.±0.5° C. The dissolution testing of the tablets was conducted over an 18-hour period. Tablet dissolution data were collected at baseline and at the 0.5-hour, 1-hour, 2-hour, 6-hour, 8-hour, 10-hour, 12-hour, 14-hour, and 16-hour time points. Data collected is shown in Table 9 below and is illustrated in FIG. 1, though FIG. 1 only shows data up to the 16-hour time point.

TABLE 9
Uncoated 10 mg Nicorandil Tablet Dissolution, pH 6.8 (n = 2)

| Time (h) | Average | SD |
|---|---|---|
| 0.00 | 0.00 | 0.00 |
| 0.25 | 12.13 | 1.15 |
| 1.00 | 28.03 | 1.63 |
| 2.00 | 42.55 | 1.66 |
| 6.00 | 76.02 | 1.63 |
| 8.00 | 84.48 | 1.34 |
| 10.00 | 89.47 | 1.42 |
| 12.00 | 91.71 | 1.34 |
| 14.00 | 92.23 | 0.89 |
| 16.00 | 91.84 | 0.74 |
| 18.00 | 90.88 | 0.75 |

As shown in Table 9 and illustrated in FIG. 1, the exemplary 10 mg uncoated, first release nicorandil tablets demonstrate a dissolution profile wherein, on average, between about 40%-45% of the formulation is dissolved at 2 hours post-dose, e.g., between about 40%-45% of the nicorandil has been released under test conditions of pH 6.8 at 2 hours post-dose. Further, by 6-hours post-dose, between about 75%-80% of the nicorandil has been released (about 75%-80% of the tablets have dissolved), and by 10 hours, about 90% of the nicorandil has been released (about 90% of the tablets have dissolved) under the provided test conditions.

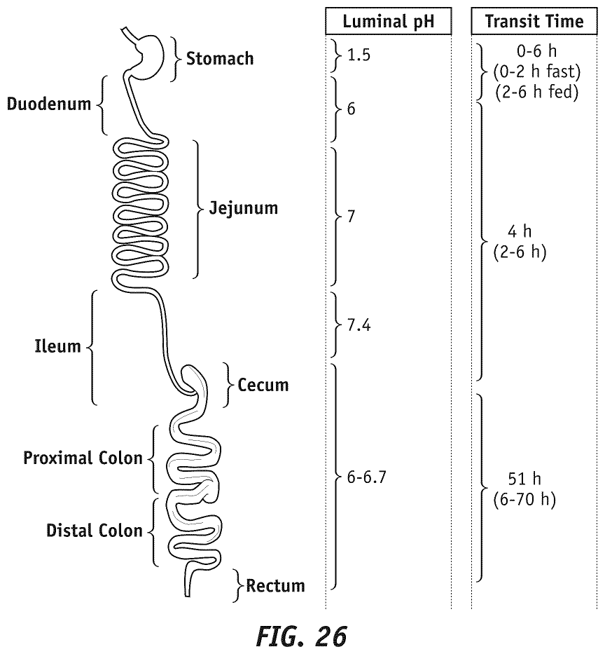

FIG. 1 graphically presents the average dissolution results of the first release nicorandil tablets under dissolution testing conditions of pH 6.8, mimicking the higher pH environment of the intestines (conditions which reflect a significantly higher (more alkaline) pH than that of earlier portions of the gastrointestinal tract such as the stomach (see also FIG. 26)). The Figure demonstrates that about 90% of the contents of the first release, uncoated tablet component(s) are released between about 10 hours and 11 hours after administration at a pH of 6.8, that of the later sections of the intestines. However, as was shown in Example 2, an uncoated, first release tablet would likely be at least substantially dissolved in the low pH environment of the stomach prior to reaching the higher pH of the later gastrointestinal tract.

### Example 3

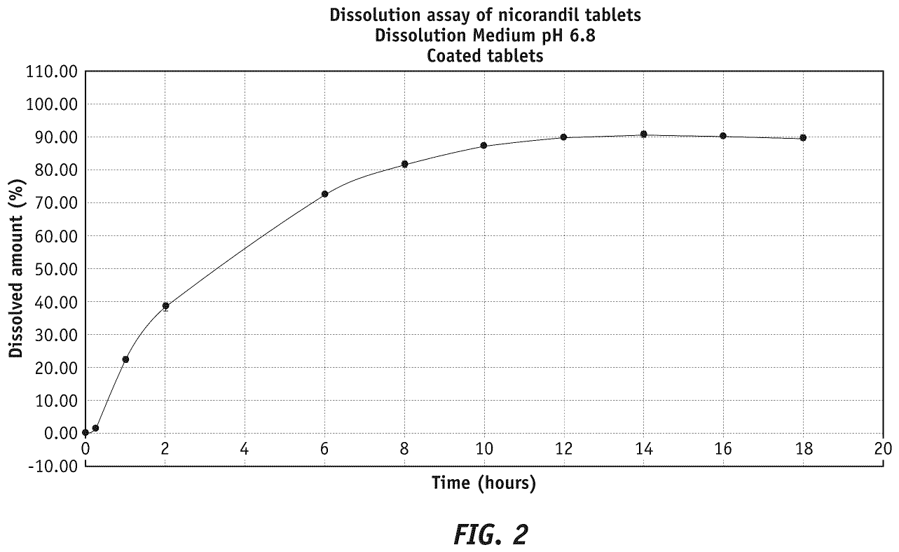

An experiment was performed to analyze the dissolution profile of coated, second release (pH responsive formulation) exemplary nicorandil tablets at pH 6.8. Individual tablets used in the experiment were manufactured according to the process described in Example 1. The formulation of tablets used in the experiment is shown in Table 8, above (represented by the formulation with the coating ingredients). A number of second release tablets were collected for dissolution testing using a USP apparatus type II system (Agilent) at 50 rpm. Dissolution testing of the capsules was performed using 900 mL of a dissolution medium having a pH of 6.8 and maintained at 37° C.±0.5° C. The dissolution testing of the tablets was conducted over an 18-hour period. Tablet dissolution data were collected at baseline and at the 0.25-hour, 1-hour, 2-hour, 6-hour, 8-hour, 10-hour, 12-hour, 14-hour, 16-hour, and 18-hour time points. Data collected is shown in Table 10 below and is illustrated in FIG. 2.

TABLE 10
Coated 10 mg Nicorandil Tablet Dissolution, pH 6.8

| Time (h) | Average | SD |
|---|---|---|
| 0.00 | 0.00 | 0.00 |
| 0.25 | 1.45 | 0.10 |
| 1.00 | 22.20 | 0.67 |
| 2.00 | 38.31 | 1.14 |
| 6.00 | 72.42 | 0.16 |
| 8.00 | 81.42 | 0.60 |
| 10.00 | 87.06 | 0.30 |
| 12.00 | 89.67 | 0.25 |
| 14.00 | 90.48 | 0.71 |
| 16.00 | 90.12 | 0.37 |
| 18.00 | 89.40 | 0.54 |

As shown above in Table 10 and illustrated in FIG. 2, 10 mg coated, first release nicorandil tablets demonstrate a dissolution profile wherein, on average, between about 35%-40% of the formulation is dissolved at 2 hours post-dose, e.g., between about 35%-40% of the nicorandil has been released under test conditions of pH 6.8 at 2 hours post-dose. Further, by 6-hours post-dose, between about 70%-75% of the nicorandil has been released (about 70%-75% of the tablets have dissolved), and by 10 hours, between about 85%-about 90% of the nicorandil has been released (between about 85%-about 90% of the tablets have dissolved). By 12 hours post-dose, on average about 90% of the uncoated tablet(s) have dissolved (e.g., about 90% of the nicorandil has been released) under the provided test conditions.

FIG. 2 graphically presents the average dissolution results of the second release nicorandil tablets under dissolution testing conditions of pH 6.8, mimicking the higher pH environment (e.g., higher than that of earlier portions of the gastrointestinal tract such as the stomach) of the later gastrointestinal tract, such as, e.g., within the intestines. The Figure demonstrates that at a pH of 6.8, the pH of the later sections of the gastrointestinal tract, e.g., past the stomach and into the intestines, the coated, second release tablets exhibit a dissolution profile such that it takes up to 12 hours for 90% of the tablets to dissolve. This demonstrates that incorporation of such tablets into an extended-release formulation can be expected to provide release of therapeutically effective amounts of nicorandil over an expanded time frame, e.g., up to about 16 hours or more post-dose of a composition comprising both first release and extended-release forms of nicorandil. This can be expected because on average it takes between about 2-5 hours for an ingested dosage form to pass through the higher pH (e.g., a pH of about 1.2) of the stomach (depending on the additional food(s) consumed by the recipient of the dosage form which may be present in the gastrointestinal tract) and to reach locations of the gastrointestinal tract having a higher pH, such as, e.g., pH 5.5 or higher. If dissolution of an extended-release tablet begins approximately 5 hours post-dose, and 90% dissolution of such a tablet takes about 12 hours, an extended-release dosage form can be expected to be releasing nicorandil at a, e.g., 17-hour time point post-dose.

### Example 4

An experiment was performed to analyze the dissolution profile of exemplary uncoated (first/general) release nicorandil tablets maintained at pH 1.2.

Individual tablets used in the experiment were manufactured according to the process described in Example 1. The formulation of tablets and capsules used in the experiment is shown in Table 8 (represented by the formulation without the coating ingredients).

Two first release tablets were collected for dissolution testing using a USP apparatus type II system (Agilent) at 50 rpm. Dissolution testing of the capsules was performed using 900 mL of a dissolution medium having a pH of 1.2 and maintained at 37° C.±0.5° C.

The dissolution testing of the tablets was conducted over an 18-hour period. Tablet dissolution data were collected at baseline and at the 0.25-hour, 1-hour, 2-hour, 6-hour, 8-hour, 10-hour, 12-hour, and 14-hour time points. Data collected is shown in Table 11 below and is illustrated in FIG. 3, though FIG. 3 only provides data up to the 14-hour time point.

TABLE 11
Uncoated 10 mg Nicorandil Tablet Dissolution, pH 1.2 (n = 2)

| Time (h) | Average | SD |
|---|---|---|
| 0.00 | 0.00 | 0.00 |
| 0.25* | 19.62 | 0.84 |
| 1.00 | 43.22 | 0.26 |
| 2.00 | 62.58 | 0.73 |
| 6.00 | 96.84 | 1.59 |
| 8.00 | 100.74 | 1.39 |
| 10.00 | 100.79 | 0.21 |
| 12.00 | 100.49 | 0.63 |
| 14.00 | 100.37 | 0.44 |
| 16.00 | 100.43 | 0.57 |
| 18.00 | 100.35 | 0.40 |

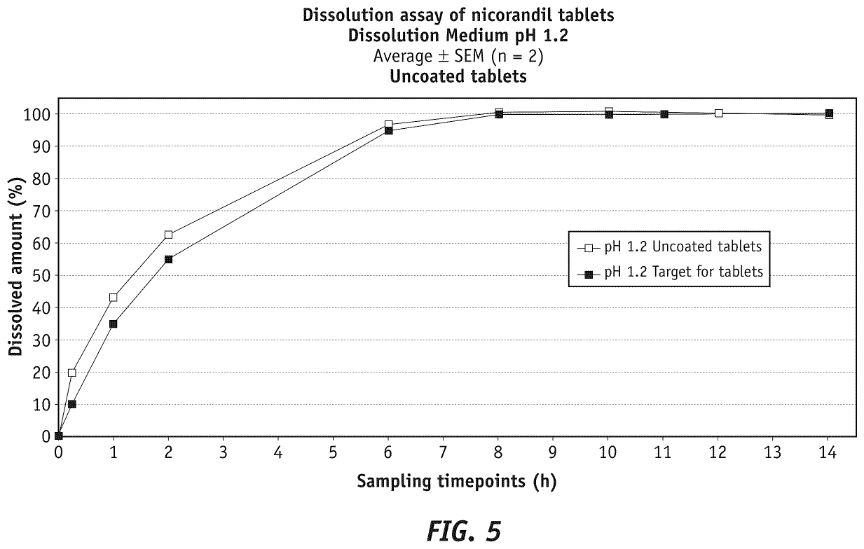

*Note that the 0.25-time point is misplaced on the graph of FIG. 3, appearing as if it is a 0.5-hour data point. This is more appropriately shown in FIG. 5.

As shown in Table 11 and illustrated in FIG. 3, 10 mg uncoated, first release nicorandil tablets demonstrate a dissolution profile wherein, on average, between about 60%-70% of the formulation is dissolved at 2 hours post-dose, e.g., between about 60%-70% of the nicorandil has been released under test conditions of pH 1.2. Further, between 5-6 hours post-dose (i.e., post contact with the applicable acidic dissolution media), about 90% of nicorandil has been released (about 90% of the tablets have dissolved, and by 6 hours, more than about 90% of the nicorandil has been released (more than about 90% of the tablets have dissolved). By ˜8 hours post-initiation/contact, about 100% of the composition has dissolved (about 100% of the nicorandil therein has been released) under these test conditions. These data indicate that it can be expected that in a combined composition of first and second release nicorandil tablets, most, such as about 85%, 90%, 95%, or about 100% of the nicorandil initially provided/contained in such a delivery form will dissolve by between about 6-8 hours post-dose. Accordingly, to achieve a longer-acting nicorandil product, e.g., product with a release profile suitable for a once-per-day administration regimen, a second release form of nicorandil should be present to provide a therapeutic effect starting at a time point of between about 4-8 or 5-8 hours post-dose.

### Example 5

An experiment was performed to analyze the dissolution profile of coated (pH responsive, second release) nicorandil tablets at pH 1.2.

Individual experimental nicorandil tablets used in the experiment were manufactured according to the process described in Example 1. The formulation of tablets and capsules used in the experiment is shown in Table 8 (represented by the exemplary formulation including coating ingredients).

Two of the coated (pH-responsive release) nicorandil tablets were collected for dissolution testing using a USP apparatus type II system (Agilent) at 50 rpm. Dissolution testing of the capsules was performed using 900 mL of a dissolution medium having a pH of 1.2 and maintained at 37° C.±0.5° C.

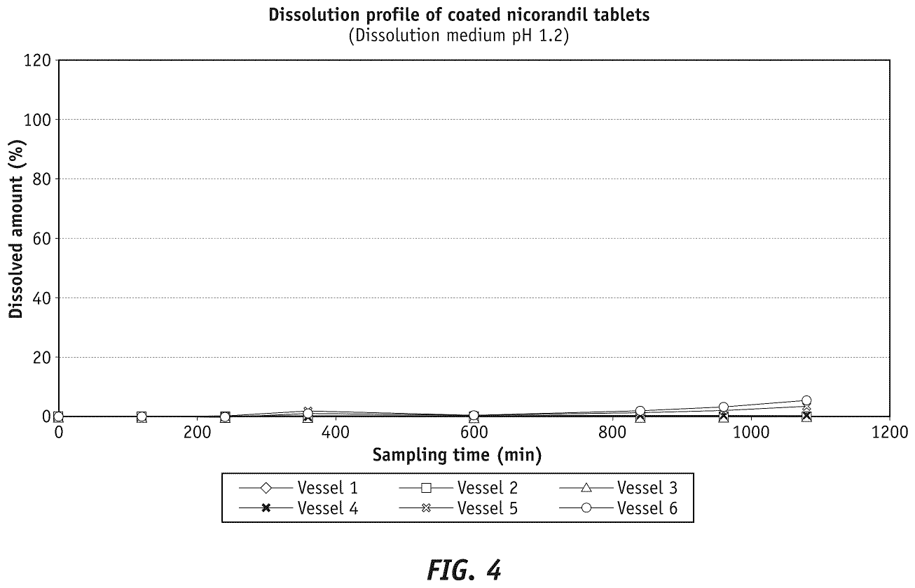

The dissolution testing of the tablets was conducted over an approximately 18-hour period. Tablet dissolution data were collected at baseline and indicated time points. Data collected is shown in Table 12 below and is illustrated in FIG. 4.

TABLE 12
Coated 10 mg Nicorandil Tablet Dissolution, pH 1.2 (n = 2)

| Time (h) | Average | SD |
|---|---|---|
| 0.00 | 0.00 | 0.00 |
| 0.25 | 0.08 | 0 |
| 1.00 | 0.105 | 0.0424 |
| 2.00 | 0 | 0 |
| 6.00 | 0.08 | 0.0424 |
| 8.00 | 0.445 | 0.219 |
| 10.00 | 0.81 | 0.02828 |
| 12.00 | 2.04 | 0.212 |
| 14.00 | 4.455 | 0.332 |
| 16.00 | 7.57 | 1.1738 |
| 18.00 | 12.2 | 1.8950 |

As shown in Table 12 and illustrated in FIG. 4, 10 mg coated, second release (pH-responsive release) nicorandil tablets demonstrate a dissolution profile essentially no nicorandil is dissolved from the formulation 1, 2, and even 6 hours post administration (e.g., in the high acidity conditions of the stomach).

FIG. 4 graphically presents the average dissolution results of the second release (pH responsive release) nicorandil tablets under dissolution testing conditions of pH 1.2, mimicking the low pH environment of the stomach. These data indicate that it can be expected that in a combined composition of first and second release nicorandil tablets, essentially 0% of the nicorandil provided in such a delivery form will dissolve by ˜4-6 hours post-administration.

### Example 6

A target release profile was generated for the dissolution of exemplary uncoated (first/general) controlled release nicorandil tablets at pH 1.2, reflecting a release profile predicted to be suitable for one part of a two-pulse release capsule formulation according to the invention (expected to be suitable for QD nicorandil delivery (e.g., for treatment of angina or other conditions described in this disclosure).

An experiment was performed to analyze the dissolution profile of uncoated nicorandil tablets produced according to the provisions of Example 1, as described above, at a pH of 1.2 according to the dissolution conditions described in the preceding Examples and the results were plotted along with the target release profile in FIG. 5.

This data presented in FIG. 5 illustrates that uncoated (general release) nicorandil tablets of the invention release nicorandil throughout the anticipated period of the tablet's residence in the stomach (continuing release of nicorandil for about 6 hours from contact with a pH 1.2 dissolution media) and that by employing the principles provided in this disclosure, those of ordinary skill in the art can prepare controlled release formulations according to various target performance specifications set forth herein.

### Example 7

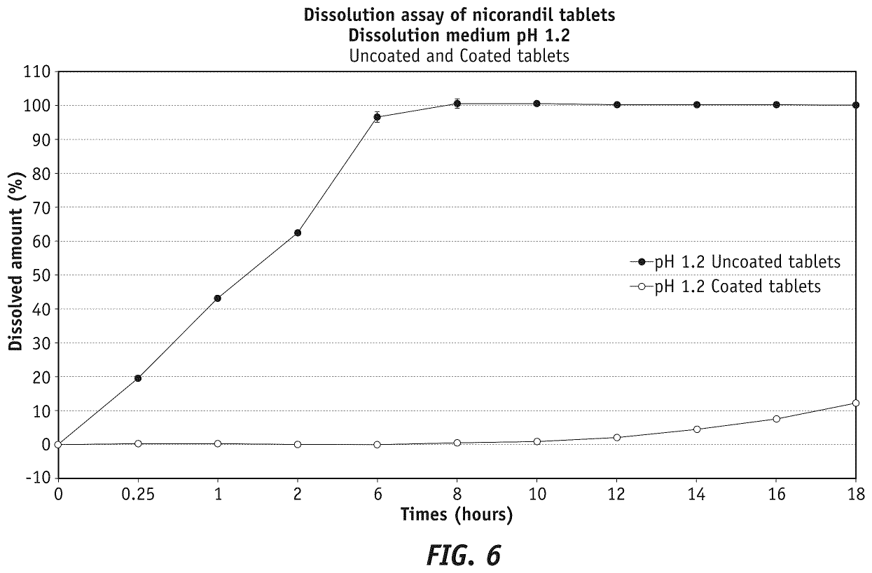

An exercise was performed to better present the characteristics of exemplary nicorandil formulations. Data from above-described experiments performed using both the exemplary uncoated nicorandil tablets and exemplary coated nicorandil tablets described above, in dissolution studies performed at pH 1.2, are plotted together in FIG. 6.

FIG. 6 clearly illustrates the different characteristics of these two different release forms (general and pH-responsive) included in compositions of the invention, which ultimately impart compositions of the invention with a two-pulse release of nicorandil from the overall formulation/dosage form. As pointed out above and in connection with similar data below, throughout the initial contact with highly acidic conditions, such as found in the stomach, the general controlled release formulation releases nicorandil in a steady manner, whereas the pH-responsive/coated dosage forms of the composition release essentially no nicorandil during such period, preserving such nicorandil until the second/pH-responsive dosage form reaches the intestine.

### Example 8

An experiment was performed to analyze the dissolution profile of an exemplary 40 mg two-pulse release nicorandil formulation in final delivery form. Specifically, the tested 40 mg nicorandil formulation included a HPMC capsule (delivery facilitating component) comprising 20 mg of nicorandil in first release form (uncoated tablets) and 20 mg of nicorandil in second release form (in pH responsive coated tablets). The 20 mg of nicorandil in first release form was present within the capsule as two 10 mg first release tablets and the 20 mg of nicorandil in second release form was present in the capsule as two 10 mg coated tablets.

Capsules used in the experiment were manufactured according to the process described in Example 1. The formulation of tablets and capsules used in the experiments are shown in Table 8 above (contained in Example 1).

Two capsules were collected for dissolution testing using a USP apparatus type II system (Agilent) at 50 rpm. Dissolution testing of the capsules was performed using 900 mL of a dissolution medium having a pH of 6.8 and maintained at 37° C.±0.5° C.

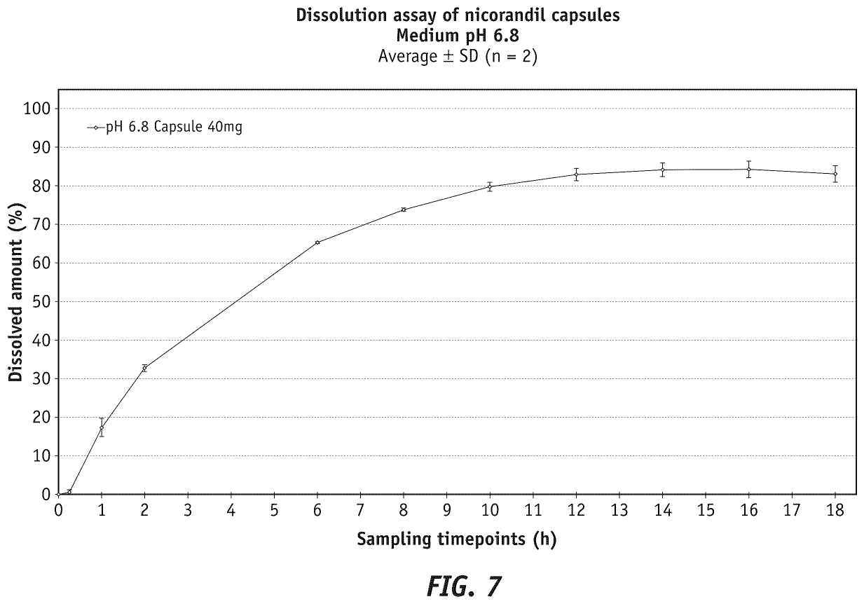

The dissolution testing was conducted over an 18-hour period. Capsule dissolution data were collected at baseline and at the 0.25-hour, 1-hour, 2-hour, 6-hour, 8-hour, 10-hour, 12-hour, 14-hour, 16-hour, and 18-hour time points. Data collected is shown in Table 13 below and is illustrated in FIG. 7.

TABLE 13
40 mg Nicorandil Capsule Dissolution, pH 6.8

| Time | Time | Capsule | Capsule | Average | |
|---|---|---|---|---|---|
| (hrs) | (mins) | 1 (%) | 2 (%) | (%) | SD |
| 0 | 0 | 0 | 0 | 0.0 | 0 |
| 0.25 | 15 | 0.03831 | 0.956921 | 0.5 | 0.649556 |
| 1 | 60 | 15.62527 | 18.90174 | 17.3 | 2.316813 |
| 2 | 120 | 32.10234 | 33.36061 | 32.7 | 0.889733 |
| 6 | 360 | 65.1152 | 65.45991 | 65.3 | 0.243743 |
| 8 | 480 | 73.62005 | 74.17363 | 73.9 | 0.391444 |
| 10 | 600 | 78.97217 | 80.63427 | 79.8 | 1.17528 |
| 12 | 720 | 81.83126 | 84.01909 | 82.9 | 1.547029 |
| 14 | 840 | 82.83854 | 85.43282 | 84.1 | 1.834432 |
| 16 | 960 | 82.76423 | 85.71445 | 84.2 | 2.08612 |
| 18 | 1080 | 81.5261 | 84.60859 | 83.1 | 2.179647 |

As shown in Table 13 and provided graphically in FIG. 7, 40 mg nicorandil capsules demonstrate a dissolution profile wherein on average about 0.5% of total nicorandil in the capsules dissolved after 0.25 hours; on average about 17.3% of the total nicorandil in the capsules dissolved after about 1 hour; on average about 32.7% of the total nicorandil in the capsules dissolved after about 2 hours; on average about 65.3% of the total nicorandil in the capsules dissolved after about 6 hours; on average about 73.9% of the total nicorandil in the capsules dissolved after about 8 hours; on average about 79.8% of the total nicorandil in the capsules dissolved after about 10 hours; on average about 82.9% of the total nicorandil in the capsules dissolved after about 12 hours; on average about 84.1% of the total nicorandil in the capsules dissolved after about 14 hours; on average about 84.2% of the total nicorandil in the capsules dissolved after about 16 hours; and on average about 83.1% of the total nicorandil in the capsules dissolved after about 18 hours at pH 6.8 under the tested conditions.

On average it takes between about 2-6 hours for an ingested dosage form to pass through the lower pH (e.g., a pH of about 1.2) of the stomach (depending on the additional food(s) consumed by the recipient of the dosage form which may be present in the gastrointestinal tract) and to reach locations of the gastrointestinal tract having a higher pH, such as, e.g., pH 5.5 or higher. A successful extended-release product should be able to demonstrate successful sustained release after reaching such a higher pH environment in order to maintain therapeutic levels of the active(s) contained therein. If dissolution of a second release tablet begins approximately 5 hours post-dose, and as demonstrated in FIG. 7 it can take up to about 16 hours to reach about 84% dissolution of the second release tablet in a high pH environment such as a pH of about 6.8 (dissolution testing conditions), incorporation of such a tablet into an extended-release product can be expected to provide for a product capable of delivering therapeutically effective amounts of nicorandil for as long as, e.g., 21 hours or longer, supporting a once-per-day administration.

### Example 9

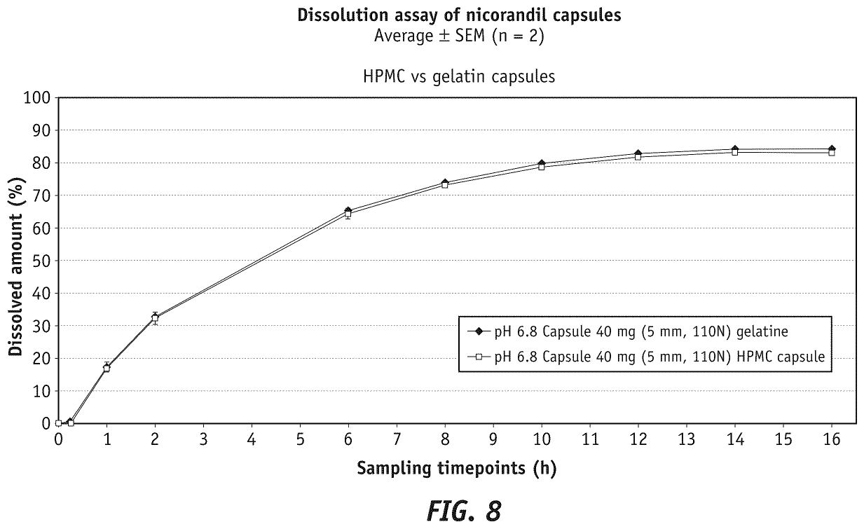

An experiment was performed to analyze the suitability of different capsule types on compositions of the invention. Both HPMC capsules and gelatin capsules containing two coated and two uncoated nicorandil tablets as described above were produced and were subjected to a dissolution assay at pH 6.8, under conditions similar to those described in earlier Experiments. The results of these studies are presented in FIG. 8.

As can be seen from FIG. 8, the difference between HPMC and gelatin capsules on the dissolution profile of the exemplary nicorandil capsules was not significant. This data exemplifies different types of nicorandil capsules can be readily used in compositions according to the invention without resulting in a significant impact on the release profile from the general release and pH-responsive release dosage forms contained within the capsule.

### Example 10

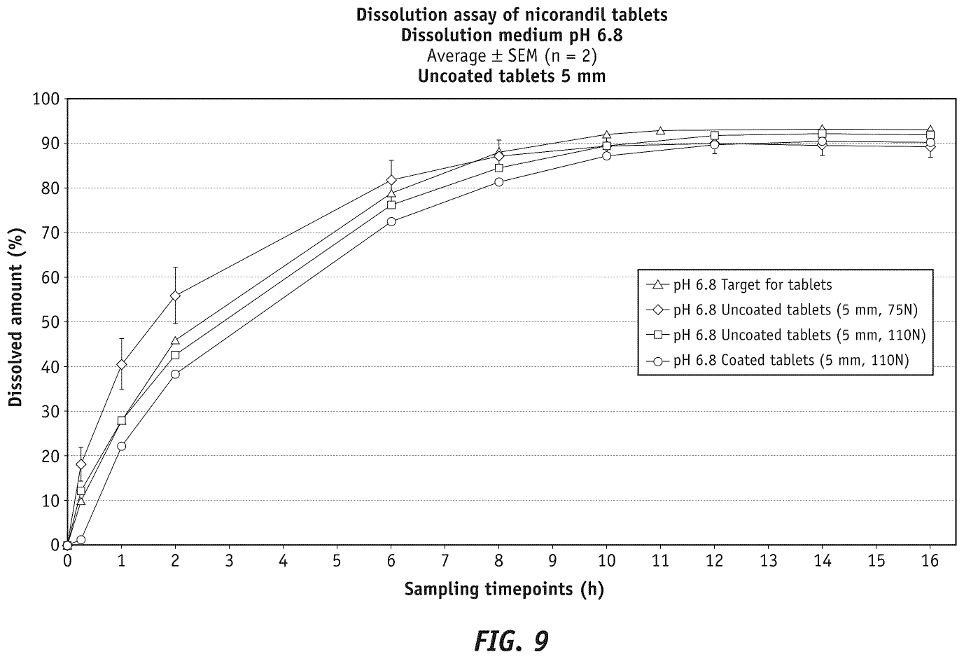

An experiment was performed to analyze the suitability of tablet hardness on the release profiles of uncoated and coated tablets. 5 mm coated nicorandil tablets with a hardness of 110, 5 mm uncoated nicorandil tablets with a hardness of 75N, and 5 mm uncoated nicorandil tablets with a hardness of 110N were each subjected to a dissolution study at pH 6.8 using conditions similar to those described above. The results are presented in FIG. 9.

As shown in FIG. 9, the uncoated 5 mm 75N nicorandil tablets showed a release profile up to 10 h at pH 6.8. A slightly slower release was observed with increased hardness/compression (of 110N) in the uncoated tablets, namely a release up to 12 h. The coated 5 mm tablets (110N) also showed a release profile up to 12 h at pH 6.8. Overall, both the coated and uncoated with hardness ˜110N showed a release that was in line with the target profile (orange). The tablets with hardness ˜75N showed a slightly faster release. This Example demonstrates that a range of tablets with different physiochemical properties can exhibit a satisfactory nicorandil release profile according to aspects of the invention. On the other hand, this Example also demonstrates even further improved characteristics can be obtained by improving the hardness of the exemplary nicorandil tablets of the invention (e.g., by using a tablet with a hardness of at least ˜100 N).

### Example 11

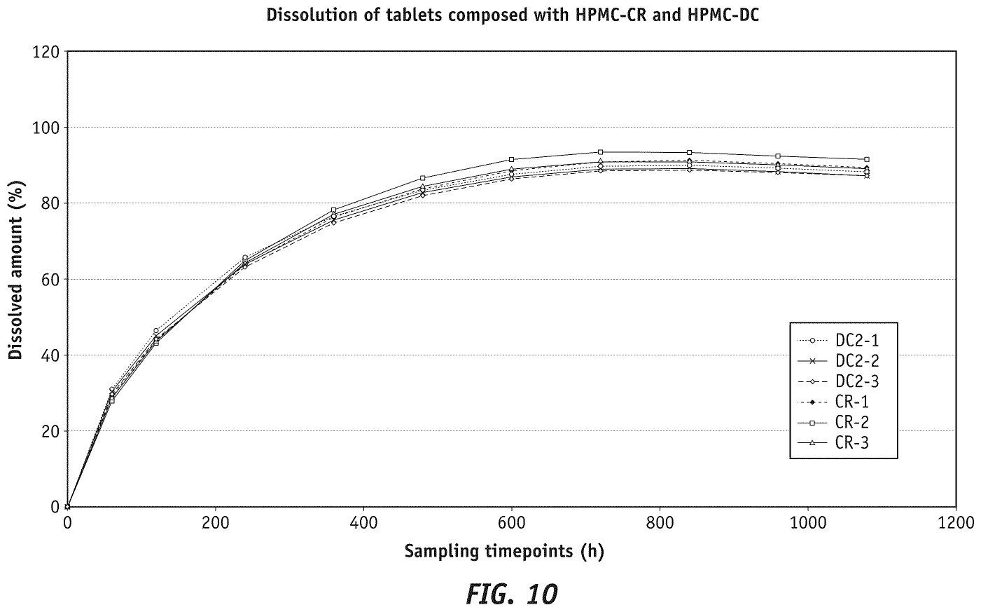

An experiment was performed to analyze the impact of inclusion of different types/grades of HPMC in the formulation of dosage forms of compositions of the invention. Nicorandil tablets were produced with either HPMC DC2 or HPMC CR K100M. Three of each type of tablet were then subjected to dissolution at a pH of 6.8. The results of the dissolution study are presented in FIG. 10 and Table 14, below.

TABLE 14
Dissolution of HPMC CR and HPMC DC2 Tablets at pH 6.8

| Sampling Time (Min) | | | | | | | | | | |
|---|---|---|---|---|---|---|---|---|---|---|
| 0 | 60 | 120 | 240 | 360 | 480 | 600 | 720 | 840 | 960 | 1080 |

HPMC CR Tablets

| | | | | | | | | | | | |
|---|---|---|---|---|---|---|---|---|---|---|---|
| Vessel 1 | 0 | 29.14 | 43.93 | 63.77 | 76.35 | 83.87 | 88.64 | 90.89 | 91.2 | 90.43 | 89.29 |
| Vessel 2 | 0 | 27.92 | 43.12 | 64.78 | 78.13 | 86.51 | 91.46 | 93.44 | 93.34 | 92.33 | 91.52 |
| Vessel 3 | 0 | 28.93 | 43.87 | 64.35 | 77.14 | 84.52 | 89.05 | 91 | 90.85 | 90.07 | 89.21 |

HPMC DC2 Tablets

| | | | | | | | | | | | |
|---|---|---|---|---|---|---|---|---|---|---|---|
| Vessel 4 | 0 | 31.07 | 46.45 | 65.77 | 76.63 | 83.49 | 87.59 | 89.64 | 90.05 | 89.26 | 88.3 |
| Vessel 5 | 0 | 30.33 | 45.14 | 64.06 | 75.61 | 82.83 | 86.97 | 88.94 | 89.11 | 88.33 | 87.23 |
| Vessel 6 | 0 | 29.69 | 44.09 | 63.32 | 74.87 | 82.02 | 86.34 | 88.44 | 88.76 | 88.05 | 87.18 |

As can be seen from FIG. 10, and Table 14, above, the inclusion of different types of HPMC in the tablet formulation did not lead to significant differences in the release of nicorandil from such tablets, reflecting flexibility of formulations of the invention in respect of such ingredients.

### Example 12

An exercise was performed to better/alternatively illustrate the performance/physiochemical characteristics of some of the exemplary nicorandil formulation components described in the preceding experiments.

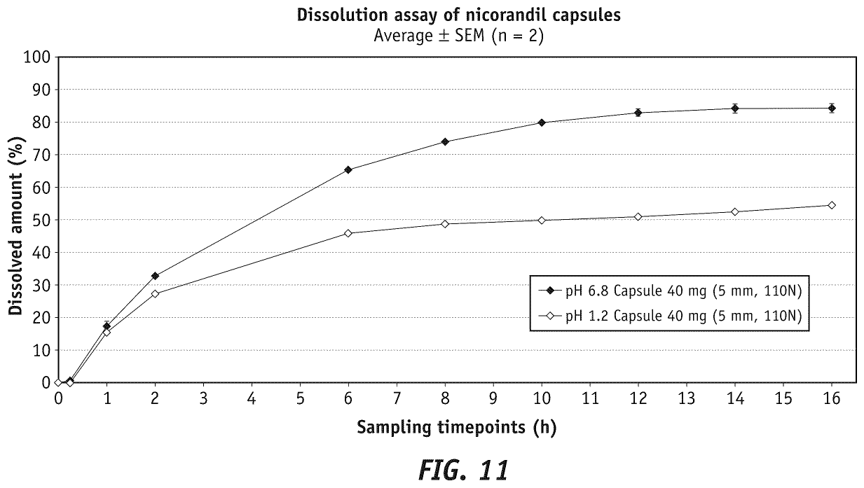

Data from above-described experiment involving dissolution an exemplary 40 mg nicorandil capsule formulation (containing 2 uncoated/general release tablets and 2 coated/pH responsive tablets) at pH 6.8 (see FIG. 7 and related discussion above) was plotted along with similar dissolution data obtained from performing a similar study on identical exemplary 40 mg nicorandil capsules at pH 1.2. The data from these different experiments is plotted together in FIG. 11 to better illustrate the differences in performance characteristics of the exemplary uncoated/general controlled release and coated/pH responsive release tablets in such a formulation.

Specifically, as can be seen from the data presented in FIG. 11, nicorandil capsules according to the invention show a complete release profile of up to 12 h-14 h at pH 6.8. In contrast, at pH 1.2 such capsules only release up to about 50% of their content within 10 h, plus ˜5% over the following 6 hours at such acidic conditions, clearly reflecting the ability of the coated tablet portion of such formulations to resist nicorandil release under acidic conditions such as are found in the stomach.

### Example 13

An exercise was performed to better/alternatively illustrate the performance/physiochemical characteristics of some of the exemplary nicorandil formulation components described in the preceding experiments.

As described above, a dissolution experiment was performed on an exemplary 40 mg nicorandil HPMC capsule formulation comprising 20 mg of nicorandil in first release form and 20 mg of nicorandil in second release form. The 20 mg of nicorandil in first release form was present within the capsule as two 10 mg first release tablets. The 20 mg of nicorandil in second release form was present in the capsule as two 10 mg second release tablets (coated tablets). Similarly, as described above, a dissolution experiment was performed to analyze the dissolution profile of 10 mg nicorandil coated (second release) tablets at pH 1.2. Individual tablets and the finished capsule used in the experiment were manufactured according to the process and formulation described in Example 1.

In the capsule dissolution study, six capsules were collected for dissolution testing using a USP apparatus type II system (Agilent) at 50 rpm. Dissolution testing was performed using 900 mL of a dissolution medium having a pH of 6.8 maintained at 37° C.±0.5° C. In parallel, 6 coated tablets, each containing 10 mg of nicorandil (notably, the same second release form present within the capsule(s) tested as a part of this Example), were collected for dissolution testing also using a USP apparatus type II system (Agilent) at 50 rpm.

Dissolution testing of the tablets was performed using 900 mL of a dissolution medium having a pH of 1.2 at 37° C.±0.5° C.

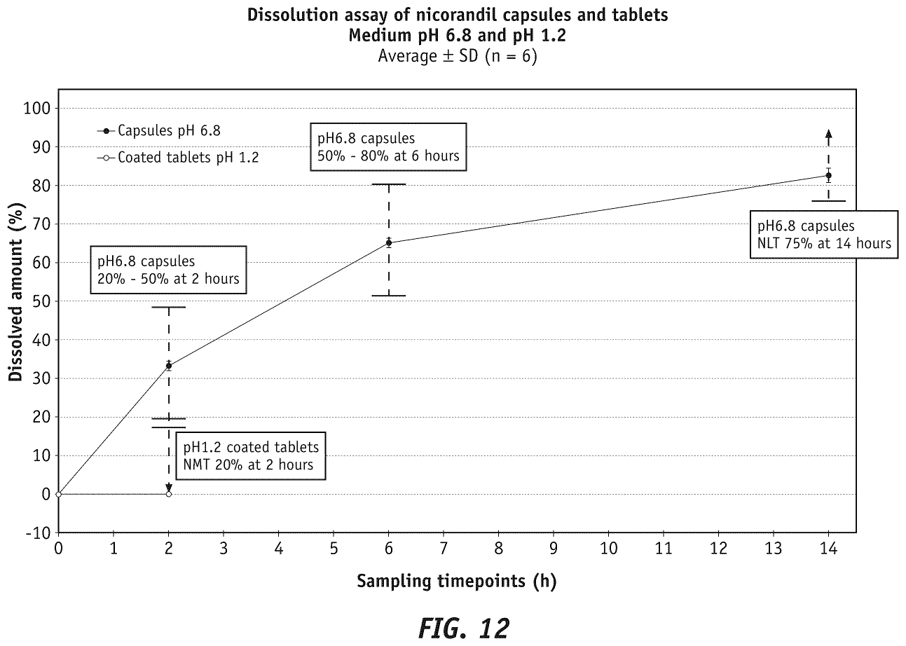

The dissolution testing of the capsules was conducted over a 14-hour period. Capsule dissolution data were collected at baseline and at the 2-hour, 6-hour, and 14-hour time points. Only data at baseline and the 2-hour time point were collected on the coated tablets. Data collected is shown in Tables 15 and 16 below and is illustrated in FIG. 12.

TABLE 15
40 mg Nicorandil Capsule Dissolution, pH 6.8

| | Time (hrs): | | | |
|---|---|---|---|---|
| | 0 | 2 | 6 | 14 |
| Capsule 1 (%) | 0 | 32.10234 | 65.1152 | 82.83854 |
| Capsule 2 (%) | 0 | 33.36061 | 65.45991 | 85.43282 |
| Capsule 3 (%) | 0 | 35.56929 | 66.53853 | 81.14865 |
| Capsule 4 (%) | 0 | 33.38955 | 63.63822 | 80.53061 |
| Capsule 5 (%) | 0 | 32.53488 | 66.05687 | 84.27062 |
| Capsule 6 (%) | 0 | 32.72003 | 63.17269 | 81.74665 |
| Average (%) | 0.0 | 33.3 | 65.0 | 82.7 |
| SD | 0 | 1.225969 | 1.334338 | 1.895635 |

TABLE 16
10 mg Nicorandil Coated Tablet Dissolution, pH 1.2

| | Time (hrs): | |
|---|---|---|
| | 0 | 2 |
| Tablet 1 (%) | 0 | 0 |
| Tablet 2 (%) | 0 | 0 |
| Tablet 3 (%) | 0 | 0 |
| Tablet 4 (%) | 0 | 0 |
| Tablet 5 (%) | 0 | 0.00345 |
| Tablet 6 (% ) | 0.004114 | 0.004114 |
| Average (%) | 0.0 | 0.0 |
| SD | 0.00168 | 0.001964 |

As shown in Table 15, 40 mg nicorandil capsules demonstrate a dissolution profile wherein, on average, about 33% of total nicorandil in the capsules dissolved after 2 hours; on average, about 65% of the total nicorandil in the capsules dissolved after about 6 hours; and on average, just over 80% of the total nicorandil in the composition dissolved after 14-hours at pH 6.8 under the tested conditions.

As shown in Table 16, only two of the six coated tablets demonstrated detectable dissolution over the two-hour time period of dissolution testing. On average, no significant dissolution of the coated tablets was observed at pH 1.2 under the tested conditions.

FIG. 12 graphically presents the average dissolution results under both test conditions. The single 2-hour time point average dissolution for the coated tablet at pH 1.2 is shown on the x-axis as 0. Tentative specifications of an acceptable product have been superimposed on the data to show what an acceptable dissolution profile might look like for capsules at pH 6.8 and coated tablets at pH 1.2 in order to provide an extended-release formulation of nicorandil that can be administered once per day. As shown, an acceptable exemplary dissolution profile of 40 mg nicorandil capsules at pH 6.8 could demonstrate a dissolution of between about 20%-about 50% at 2 hours; between about 50%-about 80% at 6 hours; and not less than (NLT) about 75% at 14 hours. Further, an exemplary acceptable dissolution profile of a 10 mg nicorandil coated tablet at pH 1.2 could demonstrate a dissolution of not more than (NMT) 20% at 2 hours.

The data provided in this Example demonstrate that at the low pH levels present early in the gastrointestinal tract (e.g., a pH of about 1.2 in the stomach), coated tablets can be expected to maintain their coating and hence not significantly contribute to the amount of nicorandil released during at least the first 2 hours post-dose. The Example demonstrates that the coating of the second release tablet is capable of preventing dissolution of a tablet comprising such coating at pH 1.2.

### Example 14

Two separate experiments were performed to mimic the behavior of the inventive 40 mg nicorandil second release formulation in the natural environment of the human gastrointestinal (digestive) tract (see, e.g., FIG. 26). The results of the two experiments were combined to conceptually demonstrate an expected dissolution profile of a combined first and second release nicorandil product. Thus this Example is a hybrid experimentally founded Example and a theoretical Example, using experimentally collected data to mimic the behavior of the tested formulations in the natural environment of the digestive tract.

A first experiment was performed to analyze the dissolution profile of a 20 mg nicorandil formulation comprising two 10 mg first release tablets. First release tablets, each comprising 10 mg of nicorandil, were manufactured according to the manufacturing method described in Example 1. The resulting tablets had a formulation according to Table 7 (meeting such formulation criteria). A number of first release tablets were collected for dissolution testing using a USP apparatus type II system (Agilent) at 50 rpm. Dissolution testing of the first release tablets was performed using 900 mL of a dissolution medium having a pH of 1.2 and maintained at 37° C.±0.5° C.

A second experiment was performed to analyze the dissolution profile of a 20 mg nicorandil formulation comprising two 10 mg delayed-release tablets (coated tablets). Delayed-release tablets, each comprising 10 mg of nicorandil, were manufactured according to the manufacturing method described in Example 1 and having a composition according to Table 7.

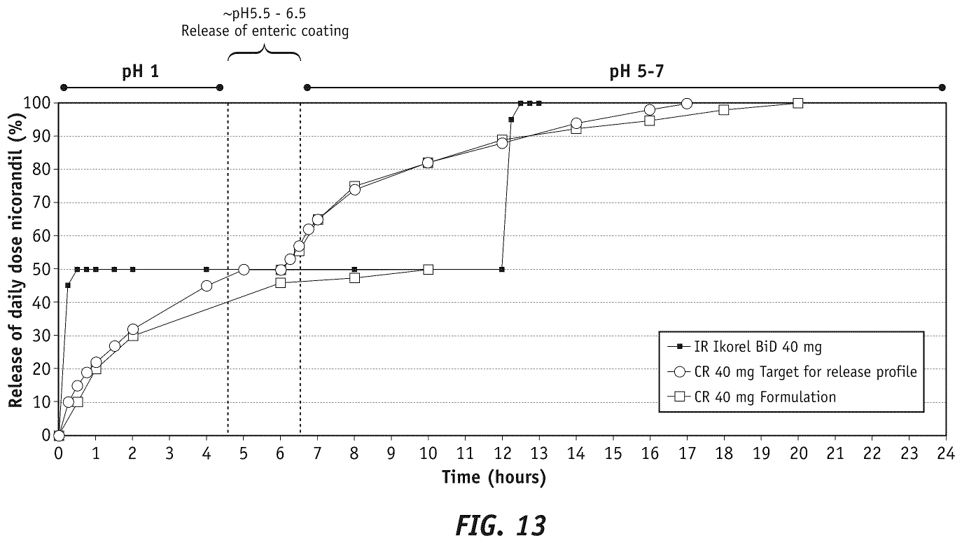

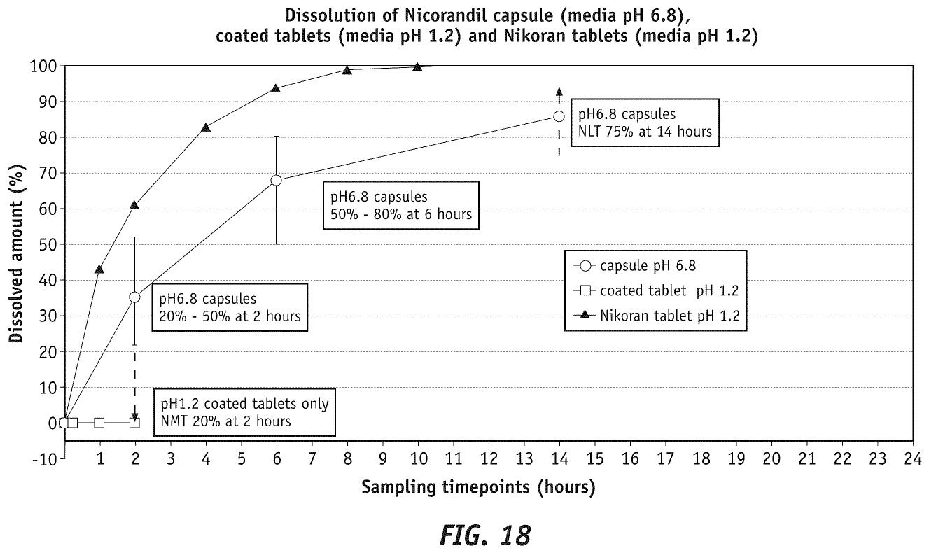

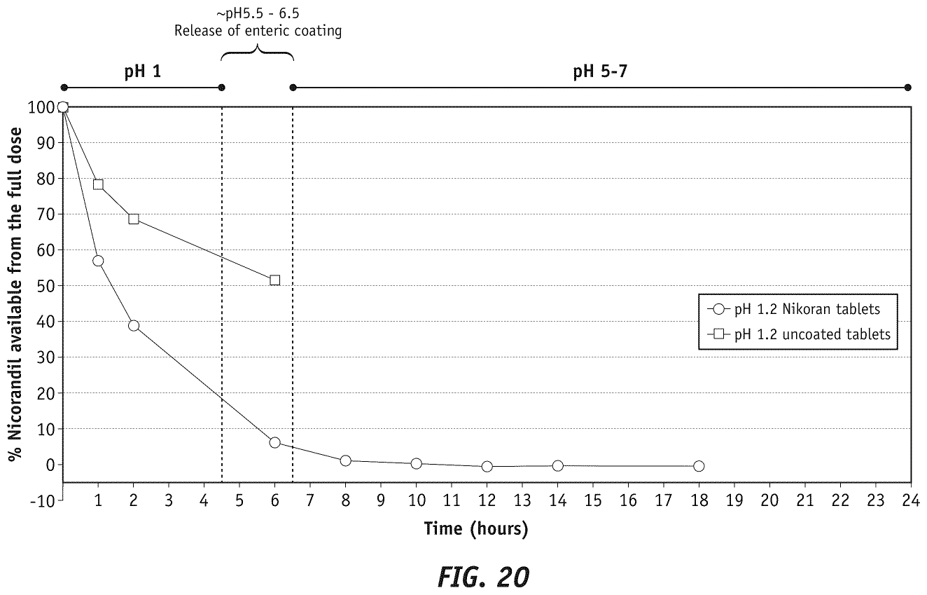

The resulting dissolution data is graphically presented in FIG. 13. Dissolution testing of the tablets in the two experiments at the different pH levels was conducted within a 14 hour-hour period; however, due to the partial theoretical nature of this experiment, FIG. 13 illustrates results on a scale spanning 24 hours. It is important in the interpretation of this Figure to understand that the delayed-release tablet dissolution testing data, collected at pH 6.8, is shown shifted such that the dissolution profile starts at about 6 hours when tablets of an orally ingested formulation would be expected to be exposed to an environment of the gastrointestinal tract having a sufficiently high pH (e.g., at least 5.5 or higher) to begin the dissolution of the coating of the delayed-release tablets. FIG. 13 shows the combined data from the 2 experiments above arranged in a combined hypothetical manner to reflect the expected in vivo performance characteristics of such a product. In other words, combined, this data represents a theoretical single formulation comprising both types of tablets, forming a single delayed-release formulation capable of providing a therapeutically effective amount of nicorandil over a delayed period of time, such as e.g., ˜20 hours or longer. Thus in this experiment, using real data, a profile was created of what would be expected to happen in the body of a consumer of this formulation. In the Y-axis of FIG. 13, it is important to understand that the 50% release mark, in reality, represents 100% of the first dose (20 mg) of the reference product Ikorel®, which is administered twice daily, yet represents only 50% of the total dose of a formulation comprising 40 mg of nicorandil distributed in both first (uncoated) dosage forms and pH responsive delayed-release tablets, which is anticipated to be administered only once daily according to aspects. Similar theoretical data presentations are made below in connection with similar comparative dissolution study results with Nikoran OD (see FIGS. 18 and 20).

Thus, the graph of FIG. 13 plots the theoretical controlled release (CR) 40 mg Formulation (represented by real data collected in the two experiments) with (1) the dissolution profile of Ikorel® twice-per-day 40 mg nicorandil on-market product (represented by the small, dark, solid squares) and (2) a target release profile for a 40 mg nicorandil formulation provided by the invention (represented by the larger, open circles). In this Example, it is the hypothesized/theoretical target profile that the CR40 mg formulation was aiming to produce.

Note that the graph in FIG. 13 has been labeled via indicators at the top of the graph to show three zones of release: a low pH release zone, labeled as “pH 1” on the graph, a zone representative of the expected environment of the stomach; a middle pH release zone, labeled as “pH 5.5-6.5” on the graph, a zone representative of the expected environment of the upper intestines within which the coating of the delayed-release tablets begins to dissolve/dissolves; and a high pH release zone, labeled as “pH 5-7” on the graph, a zone representative of the expected environment of later portions of the intestine. Importantly, these zones are indicated at the time points at which an orally delivered composition is expected to pass through each zone. Thus, in combining the data from the two experiments to demonstrate a theoretical profile of a complete 40 mg nicorandil composition comprising 20 mg of first release nicorandil (2 10 mg first release nicorandil tablets) and 20 mg of delayed-release nicorandil (2 10 mg delayed-release nicorandil tablets), the experimental dissolution data collected on the delayed-release formulation at pH 6.8 is shifted to the right, to a point in time on the graph when the composition would be expected to encounter such higher pH conditions, e.g., pH of 5.5 or higher—about 5-6 hours.

As shown in FIG. 13, after 2 hours at a low pH (pH 1.2 as utilized in the first release tablet dissolution test), about 60% of the uncoated, first release tablet nicorandil was released. As indicated by the Y-axis of FIG. 13, this represents about 30% of a complete formulation comprising 40 mg nicorandil in two portions: 20 mg first release and 20 mg delayed release. At about 6 hours, about 90% of the uncoated, first release tablet nicorandil was released. This represents about 45% of a complete formulation. By 10 hours, as shown in FIG. 13, about 100% or all of the nicorandil in the first release tablets was released, representing 50% of the nicorandil in a complete formulation.

At about the 6-hour time point post-dose, an orally administered complete dosage form would expect to encounter the higher pH of the upper intestines. At this pH (an expected pH of between about 5.5-6.5), the delayed-release coating would be expected to begin to dissolve. Thus the dissolution testing data of the delayed-release tablets at pH 6.8 is presented starting at the 6-hour time point, the point in time at which theoretically the delayed-release tablets would be exposed to an environment in the gastrointestinal tract having a pH high enough to begin dissolving the enteric coating of the tablets. As shown in FIG. 13, at about 6.5 hours, the coated, delayed-release tablets (tested at pH 6.8) are shown as being expected to have released about 10% of their nicorandil content, thus contributing to a total nicorandil release of a complete formulation of about 60% at that time point. At 8 hours, while the dissolution testing at pH 1.2 of the uncoated tablets demonstrated near-complete dissolution of the first release nicorandil, dissolution testing of the coated tablets demonstrates that about 50% of the nicorandil in the delayed-release tablets will have been released, representing about 75% of the total nicorandil in a complete formulation. By 12 hours, based on the dissolution profile of the delayed-release tablets at pH 6.8, it would be expected, and is shown in FIG. 13, that close to about 80% of the nicorandil in the delayed-release tablets will have been released, representing close to about 90% of the total nicorandil in a complete formulation (again, for the sake of clarity and to aid in understanding, the 12-hour time point of data indicating the dissolution of the delayed-release tablet represents dissolution data taken at the 6 hour time point of that dissolution experiment). At about 20 hours, based upon the dissolution profile of the delayed-release tablets at pH 6.8, it would be expected that close to all or all of the nicorandil in the delayed-release tablets will have been released, as is shown by FIG. 13, representing close to all or all of the nicorandil in a complete formulation.

As shown by the graph in FIG. 13, the combination of the two experiments resulting in a conceptual presentation of the expected dissolution profile of a combined product administered to a human recipient fits well with the proposed CR40 mg target release profile. Further, as shown in FIG. 13, the resulting profile from such a combined formulation is expected to be quite different from that of a twice-per-day administration of the first release Ikorel® formulation provided for comparison. The dissolution profile of the inventive formulations tested and demonstrated in this Example provides a profile much closer to a linear profile over a single administration period than that of the twice per day Ikorel® formulation, wherein a long plateau in dissolution is observed between doses. At each dose, the reference product Ikorel® releases nearly 90% of the active ingredient within about 15 min in pH 1.2 and pH 6.8. The proposed composition(s), however, releases nearly 90% of its first 20 mg of nicorandil (of a total 40 mg in the complete composition), present in uncoated, first release tablets, in about 9 hours at pH 1.2 (mimicking the acidic environment of the stomach), and about 90% of its second 20 mg of nicorandil (of the total 40 mg in the complete composition), present in coated, delayed-release tablets, in about 12 hours at pH 6.8 (mimicking the later sections of the intestines).

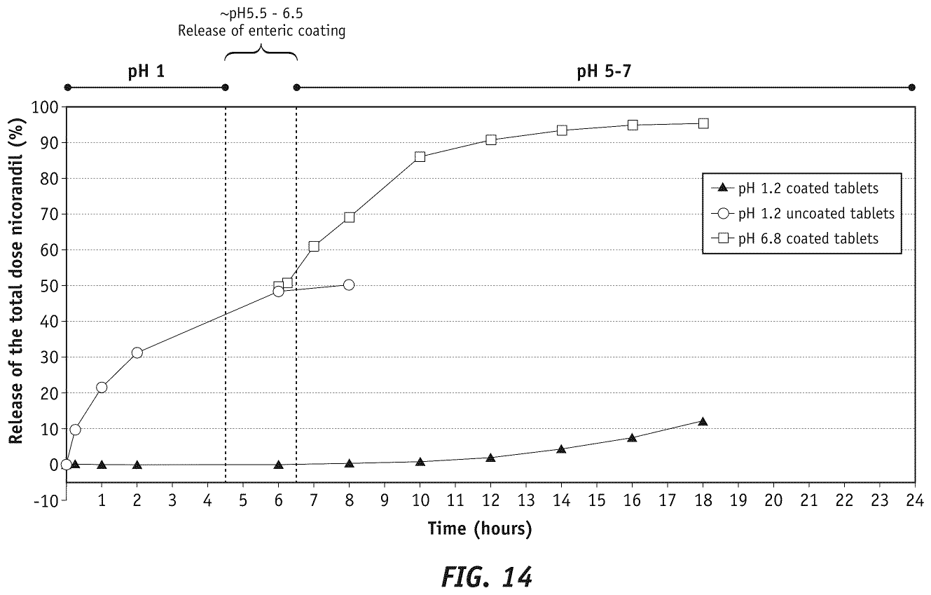

For ease of understanding, a similar illustration of data was generated without the inclusion of the Ikorel® data shown in FIG. 13 (as FIG. 14).

### Example 15

An additional exemplary nicorandil controlled release formulation was generated following the steps set forth in FIG. 21. As reflected in FIG. 21, initial ingredients (nicorandil, starch, HPMC, MCC) were provided, weighed, sieved, and mixed. Magnesium stearate (sieved) was mixed in. The combined ingredients were then subjected to tableting. For coated tablets, a coating of Eudragit in combination with PlasACRYL®, water, and colorant, was applied. Tablets were then subjected to drying, encapsulation, and packaging, using methods similar to those described above or otherwise known in the art. The composition of the additional exemplary formulation is as set forth in Table 17 (tablet formulation) and Table 18 (coating formulation).

TABLE 17
Composition of Additional Exemplary Formulation
Tablet Manufacturing

| Component | % Blend | Unit Weight (mg/tab) | Lot weight (g) |
|---|---|---|---|
| Nicorandil | 10.00 | 10.00 | 55.00 |
| MCC | 34.50 | 34.50 | 189.75 |
| Starch Starlab | 20.00 | 20.00 | 110.00 |
| HPMC K100M CR | 35.00 | 35.00 | 192.50 |
| Magnesium stearate | 0.50 | 0.50 | 2.75 |
| Total | 100.00 | 100.00 | 550 |

TABLE 18
Composition with Coating of Additional Exemplary Formulation
Coating of Tablets

| Component | % Unit Weight | Unit weight (mg/tab) |
|---|---|---|
| Core tablets (Table | 92.91 | 100.00 |
| Eudragit L 30 D-55 | 6.05 | 6.66 |
| Plasacryl HTP20 | 1.03 | 1.13 |
| Colorant | 0.01 | 0.01 |
| Water (removed | N/A | N/A |
| during processing) | | |

Such formulations were subjected to dissolution studies as described below, demonstrating that additional formulations of the invention can be generated that exhibit two-pulse nicorandil release and other characteristics according to the principles of the invention set forth in this disclosure.

### Example 16

A series of additional dissolution studies were performed to analyze the dissolution profile of nicorandil tablets and capsules according to the invention (having the composition described in Example 15) as compared to NIKORAN® OD. Nikoran® is the trade name of nicorandil tablets sold in India by Torrent Pharmaceuticals Ltd in India. Although labeled “OD,” presumably for “once daily,” the tablets actually are prescribed for twice daily administration.

Nikoran 10 mg (OD 10) tablets (Nicorandil Prolonged-Release Tablets I.P.—Batch number C820G017) were tested for their release profile in pH6.8 and pH1.2, under the conditions described above, and compared against dissolution studies of nicorandil tablets and capsules, as specified below.

The pH 1.2 dissolution media was generated by the following steps: Dilute 8.18 mL of HCl 37% in per 1 L. Prepare in a volumetric flask made up to volume with distilled water. If needed, set the pH to 1.2.

The pH 6.8 dissolution media was generated by the following steps: USP24: Dissolve 68.05 g KH2PO4 and 8.96 g NaOH in distilled water in a 10 L volumetric flask. If needed, adjust pH with HCl or NaOH. Make up to volume with distilled water.

Nicorandil standard for 40 mg capsule formulations was generated by transferring 44 mg of nicorandil API (batch NRL3002022) into a 100 mL volumetric flask. The API was dissolved and made up to volume with dissolution medium. This was diluted by transferring 2 mL to a 20 mL volumetric flask made up to volume with dissolution medium.

Nicorandil standards for 1 tablet (10 mg) for Apparatus II (0.011 mg/mL) was generated as follows: 11 mg of nicorandil API (batch NRL3002022) was transferred into a 100 mL volumetric flask. The API was dissolved and made up to volume with dissolution medium. This was diluted by transferring 2 mL to a 20 mL volumetric flask made up to volume with dissolution medium.

The conditions for mobile phase HPLC short assay: 10 mM ammonium formate was mixed in 90:10 acetonitrile and 10 mM of ammonium formate (NH4COOH) was mixed in MilliQ (pH3.0). These compositions were mixed in 90:10 ration with acetonitrile.

Apparatuses used were: Alliance Waters HPLC e2695 system, Dissolution Apparatus II (Agilent) with paddle and automatic sampler and temperature-controlled water bath, pH meter (Mettler Toledo SevenCompact)+pH electrode (Mettler Toledo Inlab Routine Pro), and Stirrer, IKA labortechnik RCT basis.

The HPLC methodology employed was as follows. The incubated samples were analyzed by HPLC using the Waters HPLC e2695 Alliance system, which consists of a vacuum degasser, a quaternary pump system, a variable volume injector, a temperature-controlled auto sampler, a temperature-controlled column manager and a photodiode array detector. The system was controlled by Empower III software (Waters). The nicorandil peak area was plotted versus time. In these tests, a Guard column was placed between the injector and the analytical column. This was done to protect the column, the HPLC system and to guarantee clean, filtered injection samples.

Assay settings were employed as follows: (a) Luna C18(2) 30 mm, diameter 4.6 mm, particle size 5 μm from Phenomenex. With filter guard. Avivia code #1279; (b) Mobile phase 10 mM ammonium formate (pH3.0) in acetonitrile in 90:10; (c) Run time: 3.5 min, and (d) Inject volume: 20 μL.

Analyses were performed duplicate, and the mean values were plotted versus time. Operating conditions included: Rotating speed paddles: 50 rpm Dissolution media: pH1.2 or pH6.8 medium, Dissolution volume: 900 mL, Temperature: 37° C.±0.5° C., Sampling volume: 1 mL, Filter sampler: 35 μm, and Timepoints: 1 h, 2 h, 4 h, 6 h, 8 h, 10 h, 12 h, 14 h, 18 h, 24 h. Reference standard per tablet 10 mg: 0.011 mg/mL and per capsules 40 mg: 0.044 mg/mL.

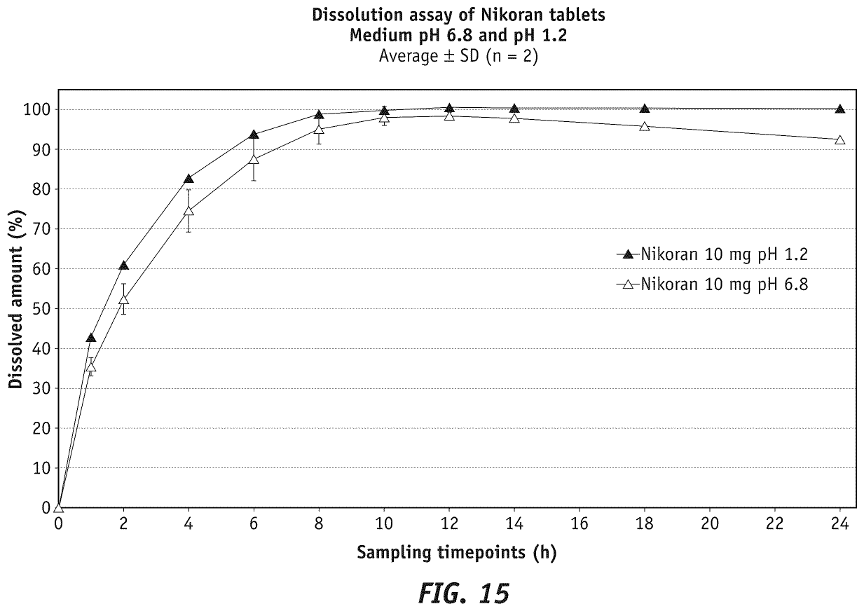

The dissolution of the tested Nikoran tablets at pH 1.2 and pH 6.8 is presented in FIG. 15 and set forth specifically in Table 19 below.

TABLE 19
DISSOLUTION PROFILE OF NIKORAN PROLONGED RELEASE
AT pH 1.2 AND pH 6.8 OVER 24 HOURS

| | | | | | | | | | | |
|---|---|---|---|---|---|---|---|---|---|---|
| 0 h | 1 h | 2 h | 4 h | 6 h | 8 h | 10 h | 12 h | 14 h | 18 h | 24 h |

NCD released during dissolution pH 6.8 (% of label claim)

| | | | | | | | | | | | |
|---|---|---|---|---|---|---|---|---|---|---|---|
| Vessel 1 | 0.0 | 37.0 | 55.1 | 78.3 | 91.2 | 97.9 | 99.4 | 99.0 | 98.1 | 95.6 | 92.4 |
| Vessel 2 | 0.0 | 33.8 | 49.6 | 70.8 | 83.7 | 92.4 | 96.6 | 97.9 | 97.4 | 96.0 | 92.7 |
| Average | 0.0 | 35.4 | 52.3 | 74.6 | 87.5 | 95.1 | 98.0 | 98.4 | 97.8 | 95.8 | 92.6 |
| SD | 0.0 | 2.3 | 3.9 | 5.3 | 5.3 | 3.9 | 2.0 | 0.8 | 0.5 | 0.3 | 0.2 |

NCD released during dissolution pH 1.2 (% of label claim)

| | | | | | | | | | | | |
|---|---|---|---|---|---|---|---|---|---|---|---|
| Vessel 1 | 0.0 | 42.8 | 61.3 | 82.9 | 93.8 | 98.8 | 99.1 | 100.0 | 100.1 | 100.2 | 100.2 |
| Vessel 2 | 0.0 | 43.1 | 61.0 | 82.9 | 93.8 | 98.9 | 100.4 | 101.0 | 100.5 | 100.6 | 100.3 |
| Average | 0.0 | 43.0 | 61.1 | 82.9 | 93.8 | 98.9 | 99.7 | 100.5 | 100.3 | 100.4 | 100.3 |
| SD | 0.0 | 0.2 | 0.2 | 0.0 | 0.0 | 0.1 | 0.9 | 0.6 | 0.2 | 0.3 | 0.1 |

As can be seen from FIG. 15, dissolution of nicorandil from Nikoran OD tablets is similar at both pH 6.8 and pH 1.2, reflecting that Nikoran tablets lack pH-responsiveness.

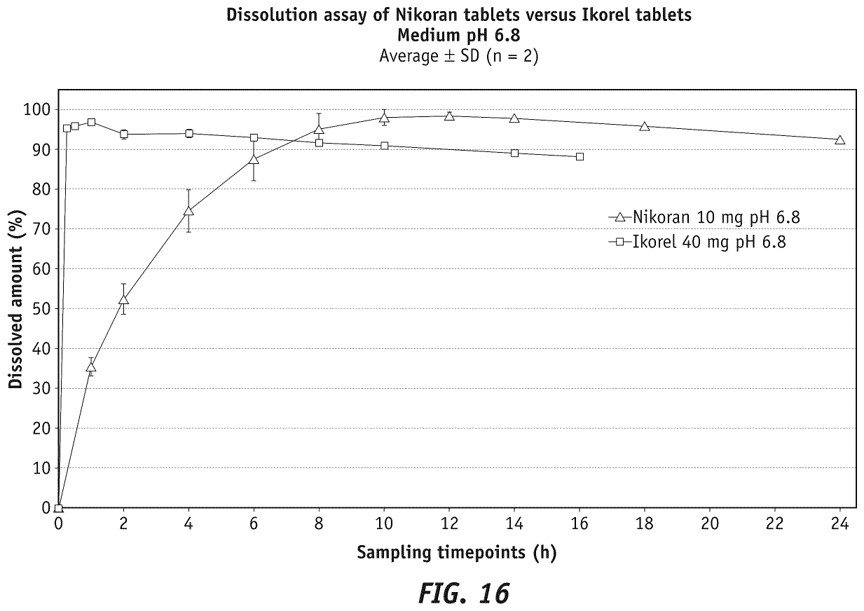

In earlier studies, reflected above, Ikorel (immediate release nicorandil tablets) were similarly tested at pH6.8. When the dissolution data for Ikorel under such conditions is plotted in the same graph as the Nikoran pH 6.8 dissolution data, the declining trend at pH 6.8 is seen in both types of commercially available formulation (FIG. 16). While the data presented in FIG. 16 clearly shows that Nikoran exhibits a sustained release, as compared to Ikorel, essentially all of the nicorandil contained in a Nikoran tablet is released at about 8 hours from contact with the pH 6.8 dissolution media.

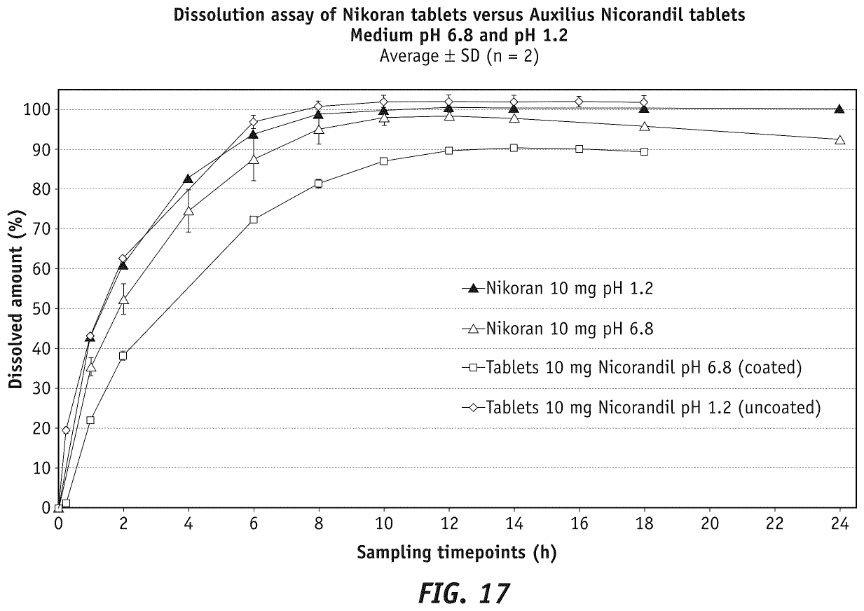

Other dissolution study runs were performed with the additional exemplary formulation tablets and capsules and the resulting dissolution data plotted in various forms. FIG. 17, for example, plots an exemplary nicorandil capsule at 6.8, coated tablets as described above at pH 1.2, as compared to the dissolution data obtained for Nikoran tablets in pH 1.2 media (as shown in FIG. 15). This data reflects the lack of dissolution of the pH responsive table/dosage form in formulations of the invention (with little or no dissolution at up to 2 hours in pH 1.2), and a more prolonged release from the exemplary nicorandil capsules as compared to Nikoran tablets. Given the similar dissolution of nicorandil from Nikoran at pH 1.2 as pH 6.8, the likely consequence is that very little to no nicorandil contained in Nikoran tablets remains in the tablet by the time the product should reach the intestine.

This data also reflects that even at pH 6.8, the general/first controlled release formulation of the invention in the tested uncoated tablets outperformed Nikoran tablets. At pH 6.8, Nikoran tablets took about 10 hours to dissolve. The test nicorandil tablets took up to 12-14 hours. Therefore, the profile of Nikoran prolonged release is clearly detectably quicker than the newly developed Nicorandil capsules.

The pH-independence of the Nikoran tablets also is clearly noticeable. The release profile of Nikoran OD looks similar in pH 1.2 as pH 6.8 (see also FIG. 15). In contrast, it is clear that the test nicorandil tablets have a clearly distinguishable pH-dependent release (pulse).

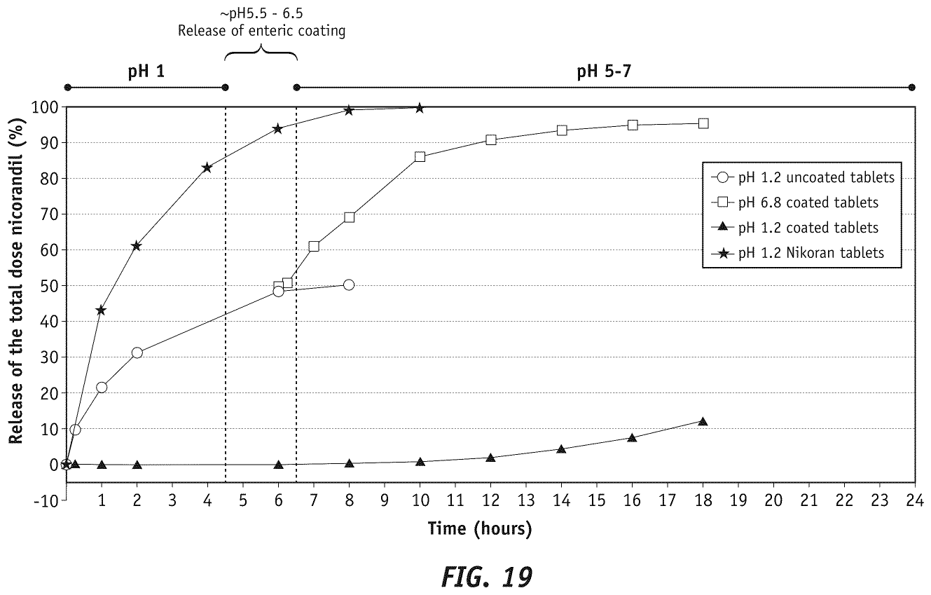

FIG. 19 provides another presentation of these data, but in a manner that models the expected in vivo release (by aligning the dissolution data for the general (first) controlled release formulation dosage form (uncoated tablets) and the coated tablets. For clarity, the dissolution data for the coated tablets at pH 1.2 is also presented. This presentation of the data clearly illustrates that the exemplary composition of the invention exhibits a substantially prolonged release of nicorandil as compared to Nikoran. E.g., based on these results at about 6-7 hours post administration it is expected that essentially all of the nicorandil would be released from Nikoran based on Nikoran's pH 1.2 dissolution data. By contrast, the exemplary composition of the invention could maintain more than 20% nicorandil 8 and 10 hours after administration, and at least 10% of the initial nicorandil of the formulation after 12 or 14 hours after administration.

To further illustrate the significance of these differences, FIG. 20 presents the expected remaining nicorandil available for absorption when released from the tablet matrix (from a full/initial dose) for both Nikoran and coated tablets of the invention at pH 1.2, reflecting the substantial difference in the pH responsive formulation of coated tablets of the inventive compositions as compared to Nikoran.

Finally, FIG. 21 combines the data from coated and uncoated exemplary nicorandil tablets at pH 1.2 and pH 6.8 conditions, in a theoretical manner (similar to as described in detail above with respect to FIGS. 13 and 14), reflecting expected in vivo performance of such compositions, and comparing such data to Nikoran. Although the presentation of such data is somewhat theoretical, this figure perhaps best illustrates that substantial difference in performance obtained by employing the inventive principles of this disclosure as compared to the only on market product described as a sustained release/once-daily nicorandil formulation. One would expect based on such data that very little or no nicorandil remains in Nikoran tablets after 7-8 hours post administration, meaning that very little (e.g., less than 10%, less than 5%, or about 1-2%) of the initial nicorandil contained in the Nikoran tablet is remaining by the time the product reaches the duodenum. In contrast, at 6 hours post administration, compositions according to the invention, such as those exemplified in these Examples, are expected to still contain at least about 40% or at least about 50% (e.g., more than 50%) of the initial nicorandil of the composition, particularly given the pH responsiveness of the second release (coated) tablets contained in the product. This contributes to the fact that formulations of the invention are expected to retain at least about 10% of the initial nicorandil (or more) at, e.g., 12 and 14 hours post administration, which, given the other characteristics of nicorandil suggest that such formulations will be effective for reduced dosing frequency regimens, such as once daily (QD) dosing regimens. In contrast, Nikoran does not appear to be suitable for such uses, which may reflect why Nikoran's label instructs twice-daily administration despite the product's “OD” branding. The changing pH conditions of the gastrointestinal tract and typical residence time of ingested products are presented in FIG. 26, to aid readers in understanding the basis for such theoretical data constructs and their basis for some of the expected in vivo and therapeutic performance characteristics the inventors envision and have set forth in this disclosure. A detailed discussion of a more complete version of this figure is provided in Hua S. Front Pharmacol. 2020 Apr. 28; 11:524.

Once again referring to the data in these figures, Nikoran exhibits a near entire dissolution in pH 1.2 media after about 6 hours. In contrast, due to the pH responsive formulation of the coated tablets of the exemplary compositions of the invention, at least 50% of the overall formulation of the invention is available and only begins to release after exposure to higher pH conditions (e.g., after about 6 hours, by which passage through the stomach and into the intestine is expected to be complete). This and other differences illustrated by FIG. 21 reflect the substantial differences between the exemplary compositions of the invention and Nikoran.

### Example 17

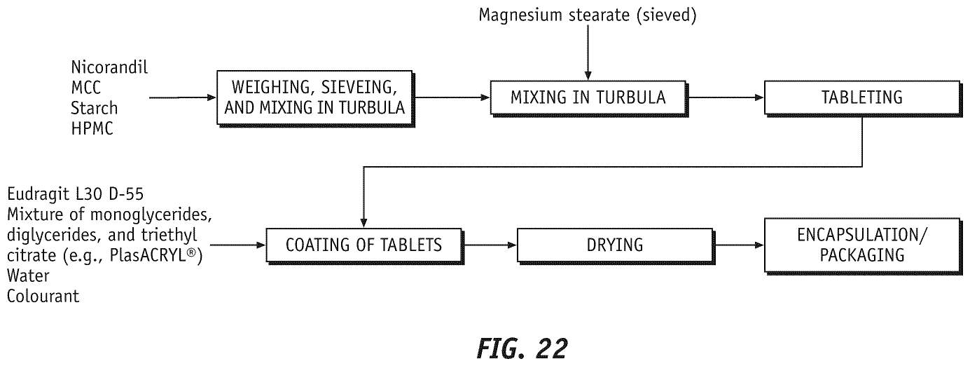

Additional studies were performed to analyze the stability of exemplary formulations provided by this disclosure. FIG. 22 presents three common impurities associated with nicorandil formulations. Impurities were observed in chromatograms of Nikoran tablets as and in exemplary Nicorandil tablets/capsules. Anther impurity considered in studies reported herein (see Example 18) is “impurity A” (imp A), which has the following chemical structure:

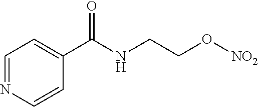 [SMILES: O=C(NCCO[N+](=O)[O-])c1ccncc1]

Impurities were consistently seen at relative retention times 0.45 and 0.6 in experimental assays. These impurities were further studied to understand if experimental nicorandil formulations were associated with a higher than typical level of impurities given the unique formulation and performance characteristics in such experimental products.

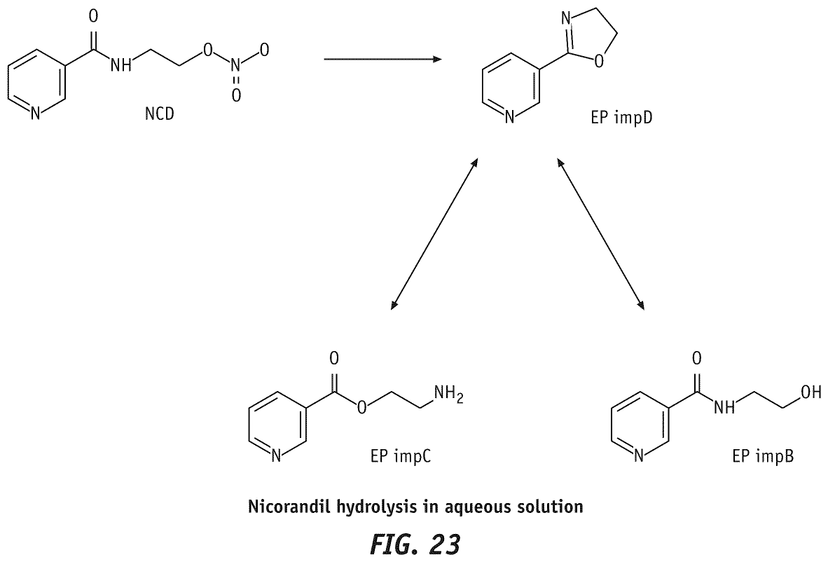

FIG. 23 presents chromatograms of Nikoran and exemplary nicorandil dosage forms overlaid in one image. The chromatograms show nicorandil (NCD) degradation after 18 h in dissolution vessel at 37° C. This data reflects that overall a similar amount of the evaluated impurities between Nikoran and the exemplary dosage forms.

Table 20 presents the level of detected impurity D (Imp D) for both the exemplary nicorandil capsules (NCD capsules) and Nikoran.

TABLE 20
(Impurity D in Experimental NCD Capsules and Nikoran Tablets)

| Imp D as % of | | | | | | | | | | | |
|---|---|---|---|---|---|---|---|---|---|---|---|
| total peak area | 0 h | 1 h | 2 h | 4 h | 6 h | 8 h | 10 h | 14 h | 16 h | 18 h | 24 h |
| Nikoran tablets | 0.0 | 1.8 | 6.4 | 5.6 | 8.2 | 7.0 | 8.1 | 9.3 | 10.4 | 12.6 | 15.7 |
| average | 0.0 | 0.0 | 3.9 | 1.9 | 3.5 | 0.0 | 0.0 | 0.0 | 0.0 | 0.1 | 0.1 |
| SD | | | | | | | | | | | |
| NCD capsules | 0.0 | 0.0 | 2.8 | 3.9 | 5.3 | | 8.2 | 9.3 | 10.3 | 12.5 | |
| average | 0.0 | 0.0 | 1.0 | 0.5 | 0.4 | | 0.2 | 0.1 | 0.0 | 0.0 | |
| SD | | | | | | | | | | | |

The relatively lower initial amounts of impurities detected from the NCD capsules as compared to Nikoran may reflect the lesser dissolution of the NCD capsules due to inclusion of the pH sensitive formulation. Thus, this data reflects that such exemplary compositions of the invention exhibit a substantially lower initial impurity level than Nikoran tablets.

### Example 18

To better understand the stability of nicorandil formulations according to the invention at an ingredient level, the following powder blend combinations were generated and analyzed:
NCD+HPMC
NCD+Starch
NCD+MCC
NCD+Magnesium stearate
NCD+Mannitol (not used in current formulation)
NCD only

In this screening, the different combinations of excipients and API (1:1 ratio) were tested at accelerated conditions (40° C./75% RH) in sealed, glass bottles to test whether the degradation is coming from moisture from the excipients. The powder blends (2.5 grams of mixture) were analyzed in duplicate and stored in sealed glass bottles at 25° C./60% RH (relative humidity) and 40° C./75% RH.

Loss of drying (LOD) values (weight percent difference) for these test compositions were determined and are presented in Table 21.

TABLE 21
(LOD Values for Nicorandil (NCD)/Excipient Mixtures)

| Mixture | LOD (%) |
|---|---|
| HPMC/NCD | 2.29 |
| Starch/NCD | 6.01 |
| MCC/NCD | 2.06 |
| Mannitol/NCD | 0.84 |
| Magnesium Stearate (Mgst)/NCD | 0.82 |
| Nicorandil (alone) | 0.28 |

These compositions were further analyzed (N=2) for impurities (Imp A, Imp B, Imp C, and Imp D) and NCD content both initially and after 2 or 4 weeks of storage at accelerated stability testing conditions (40° C.) (per US FDA typical standards/procedures) or room temperature conditions (25° C.). Unknown impurities (“Unk. Imps.”) were also measured and the sum of all measured impurities (“Sum Imps.”) calculated. The average measures from these studies are presented in Table 22, below.

TABLE 22
Weight Percent Impurities and Nicorandil Content in Excipient/NCD
Mixtures(Initially and after Storage)

| | | Imp | Imp | Imp | Imp | Unk. | | Sum | Mass |
|---|---|---|---|---|---|---|---|---|---|
| | | B | C | D | A | Imps. | NCD | Imps. | Balance |
| T = 0 | NCD | 0.01 | 0.01 | 0.11 | 0.00 | 0.06 | 98.40 | 0.20 | 98.60 |
| | Starch/NCD | 0.02 | 0.00 | 0.11 | 0.00 | 0.05 | 97.90 | 0.19 | 98.08 |
| | Mannitol/NCD | 0.02 | 0.00 | 0.12 | 0.00 | 0.06 | 103.90 | 0.19 | 104.09 |
| | HPMC/NCD | 0.02 | 0.00 | 0.15 | 0.00 | 0.06 | 98.52 | 0.23 | 98.74 |
| | MCC/NCD | 0.03 | 0.02 | 0.18 | 0.00 | 0.16 | 100.20 | 0.38 | 100.58 |
| T = 2 | NCD | 0.04 | 0.05 | 0.22 | 0.00 | 0.18 | 99.52 | 0.49 | 100.01 |
| weeks, | Starch/NCD | 0.26 | 0.34 | 0.37 | 0.00 | 0.66 | 91.01 | 1.63 | 92.64 |
| 40° C. | Mannitol/NCD | 0.07 | 0.09 | 0.30 | 0.00 | 0.52 | 114.15 | 0.98 | 115.13 |
| | HPMC/NCD | 1.66 | 2.75 | 0.00 | 0.00 | 8.57 | 81.00 | 12.99 | 93.99 |
| | MCC/NCD | 1.35 | 2.27 | 1.12 | 0.00 | 4.78 | 73.08 | 9.52 | 82.61 |
| T = 4 | NCD | 0.00 | 0.00 | 0.12 | 0.00 | 0.17 | 108.13 | 0.29 | 108.41 |
| weeks, | Starch/NCD | 0.00 | 0.00 | 0.15 | 0.00 | 0.17 | 106.73 | 0.32 | 107.05 |
| 25° C. | Mannitol/NCD | 0.00 | 0.00 | 0.13 | 0.00 | 0.19 | 105.34 | 0.32 | 105.66 |
| | HPMC/NCD | 0.03 | 0.20 | 0.44 | 0.00 | 0.33 | 103.71 | 1.01 | 104.72 |
| | MCC/NCD | 0.05 | 0.61 | 0.60 | 0.00 | 0.58 | 100.63 | 1.84 | 102.47 |

From the data presented above it can be seen that all combinations of excipients/API seemed to be stable for at least 4 weeks at 25° C./60% RH. After 2 weeks at 40° C./75% RH, the starch, HPMC, and MCC samples showed a decreased assay value. At 25° C./60% RH, impurity D was formed, while in the more severe condition of 40° C./75% RH also impurities B and C were formed. This follows the hydrolysis of nicorandil when in solution (FIG. 23). Impurity D is the primarily formed degradation product. In more severe conditions, Impurity D is hydrolyzed into degradation products B and C in equilibrium (see FIG. 23).

The nicorandil degradation observed in the formulation is assumed to come from exposure to moisture. The individual starch, individual HPMC, and individual MCC seem to have an impact on the stability of nicorandil. The intrinsic moisture content of these three excipients could explain the nicorandil degradation. At future stability studies at 25° C., especially the production of Impurity D will need to be monitored closely.

Based on these results, the inventors conceive that most, generally all, substantially all, or all excipients of formulations of the invention, the excipient content of the formulation or parts of the formulation (e.g., the body of a tablet), or both, will have a moisture content of about 3% or less, such as 2.8% or less, 2.5% or less, or 2.3% or less, such as most or generally all having a moisture content of about 2% or less, and all having a moisture content of less than 3% or less than about 2.5%.

In an exemplary formulation, the contribution to the formulation (on a weight basis) is 20% for Starch Starlab, 34.5% for MCC Prosolv HD90, and 35% for HPMC DC2. As exemplified by other results presented here, such a formulation provides dissolution profile characteristics at or near target. Accordingly, retention of these excipients is suitable. Skilled persons should note that replacement of excipients is possible if similar or better dissolution is obtained, but that, in aspects, similar or better moisture content of excipients also should be factored in (by having about the same level of impurities or less or a significantly similar or significantly improved impurity profile). The inventors further conceive that improved packaging as described elsewhere in this disclosure can detectably or significantly improve on the stability performance of excipients and overall formulations (e.g., improving on the results reported in Example 19, immediately below).

### Example 19

To further investigate stability characteristics of nicorandil formulations, additional stability studies were performed using exemplary controlled release nicorandil capsule formulations (40 mg) having the characteristics described in Example 15.

The exemplary nicorandil CR capsules were initially stored in sealed black plastic bags at approximately room temperature for approximately 3-4 weeks before the initiation of the stability study. For the purpose of the stability study, capsules were transferred to high-density polyethylene (HDPE) containers with 2-6 grams of a desiccant in the lid and were maintained in such containers during the study. HDPE is a leading pharmaceutical container material used widely in view of its advantageous stability and lack of reaction with drug products.

One sample (each sample containing two exemplary 40 mg nicorandil capsules) was subjected to storage at 5° C., another sample was maintained at 25° C./60% RH, and still another sample was maintained under FDA accelerated stability test conditions (40° C./75% RH) for a month (samples were stored in 30 capsule containers). Nicorandil assays were then performed on the capsules maintained under such conditions. The result of these assays is presented in Table 23.

TABLE 23
Percent Nicorandil Assayed from
NCD Capsules in Stability Studies

| Exposure/Conditions | Assay % (NCD) |
|---|---|
| 5° C. | 91 |
| 25° C./60% RH | 80 |
| 40° C./75% RH | 1.5 |

After the month period, nicorandil capsules were stored at 5° C. for an additional 4 weeks in sealed aluminum bags with a silica desiccant and the capsules tested for nicorandil content. The results of these experiments are presented in Table 24.

TABLE 24
Percent Nicorandil Assayed from NCD Capsules Stored
in Aluminum Packaging with Silica Desiccant at 5° C.

| Exposure/Conditions | Assay % (NCD) |
|---|---|
| 25° C./60% RH | 91.5 +/ 3 |
| 40° C./75% RH | 73 +/− 5 |

The results from these experiments reflect the fact that generating a stable nicorandil formulation is not without difficulty. None of the initial results of the stability studies performed in HDPE containers with desiccant were deemed satisfactory and only the samples stored under refrigerated condition appeared potentially capable of achieving suitable stability. Nikoran OD is also labeled to be stored under refrigerated conditions and, even under such conditions, is indicated to be usable for only a period of 5 days from initial use.

The improved results obtained by use of aluminum packaging combined with a silica desiccant indicate that either or both approaches can be used to improve the stability performance of exemplary nicorandil formulations of the invention.

The inventors have conceived of the use of more effective moisture-wicking/desiccant packaging/stability technology being combined with the exemplary nicorandil controlled release formulations described in this disclosure. An aluminum-aluminum blister pack specifically fitted with at least one layer of desiccant-fitted/coated aluminum foil (e.g., Dessiflex Plus and Dessiflex Ultra packaging available from Amcor (Zurich)) likely in combination with a regular aluminum foil layer in the blister packing. The inventors conceive that applying this type of packaging or a similarly effective wicking/anti-moisture packaging will result in nicorandil stability that is significantly better than that achieved in the studies reported above, such as at least 95%, at least 98%, at least 98.5%, or at least 99% stability under refrigerated conditions, room temperature conditions, accelerated stability conditions, or a combination of some or all thereof, over a period of, e.g., 1, 2, 3, 6, 9, 12, 18, 24, 30, or 36 months under suitable relative humidity conditions (per prevailing regulatory authority/industry stability testing standards).

### Example 20

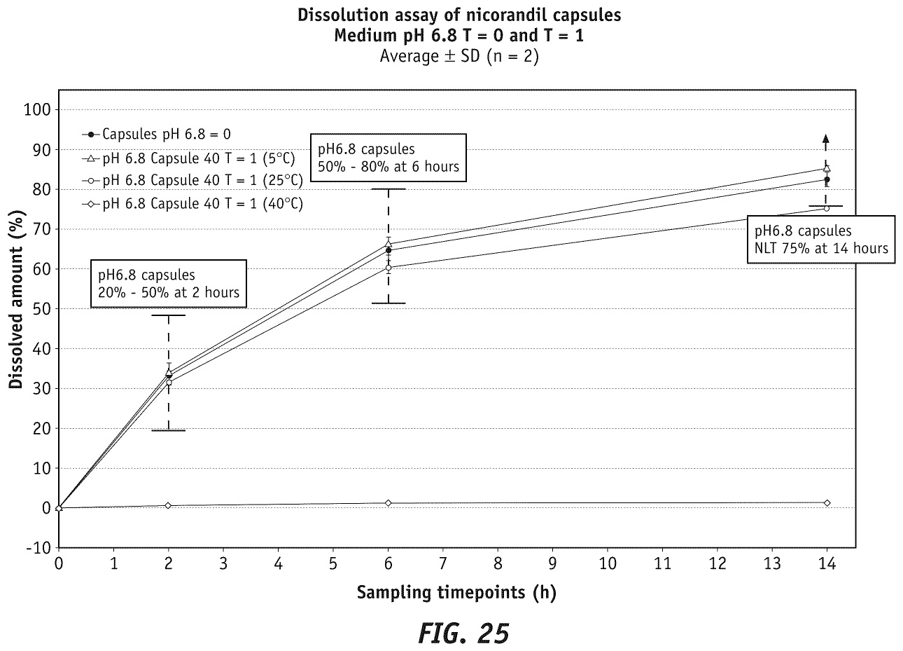

To assess the impact of storage conditions of the dissolution characteristics of exemplary nicorandil formulations, the following experiment was performed. Exemplary nicorandil capsules were subjected to both initial dissolution studies and dissolution studies after one month of storage at indicated storage conditions described above in Example 19 (at 5° C., at 25° C., and at 40° C.), in both cases at pH 6.8. The results of these dissolution studies are presented in FIG. 25.

As can be seen from the results presented in FIG. 25, in general differences in stability conditions did not significantly impact the dissolution profile of the exemplary nicorandil capsules described herein. Initially tested capsules, capsules stored at 5° C. and capsules stored at 25° C. all showed a similar pattern of dissolution at pH 6.8, although the amount of nicorandil was lower after storage, particularly at 25° C., consistent with the results described in Example 19. Also consistent with the results presented in Example 19, the capsules stored at 40° C. contained almost no nicorandil and, accordingly, a dissolution profile was not observed for such products. These results demonstrate that the dissolution pattern obtained from two-pulse nicorandil controlled release formulations according to the invention exhibit a dissolution profile suggestive of a sustained release suitable for once-daily formulations even over a range of different stability conditions.

### Example 21 (Prophetic Clinical Trial)

To demonstrate the pharmaceutical suitability of exemplary two-pulse controlled release nicorandil capsules as exemplified in Examples 1-20, the following one or more clinical study or studies including most or generally all of the following elements (or substantially similar steps/measures) can be performed.

A phase 1 type study of such products can be performed to establish tolerability/safety and other characteristics of two-pulse controlled release nicorandil products of the invention. Possible key design characteristics of such a study include sampling and PK assessment of developed dose strength(s) after both single-dose and multiple day use vis-à-vis the immediate-release reference product.

Further, pharmaceutical studies also or alternatively can include, e.g., a single-center, non-randomized, open-label, steady-state, single fixed-sequence, 3-period, 3-treatment, sequential comparative study in healthy adult volunteers that can be performed using, e.g., 3 different treatments as exemplified here:
1. Treatment A: ER Nicorandil Oral Capsule, 20 mg (fasted) QD
2. Treatment B: ER Nicorandil Oral Capsule, 40 mg (fasted) QD
3. Treatment C: IR nicorandil oral tablets, 20 mg (fasted) BID

Key Phase 1 characteristics can be described in the following way: For a multiple-dose (5 days) one treatment with Nicorandil QD 20 mg in a fasted state starting on day 1 and ending on day 5 with PK sampling on day 5 to evaluate PK parameters at steady-state of the nicorandil QD 20 mg capsule. A two-day washout follows the sampling. The same treatment group (the same cohort) continues to day 8 when participants receive a single dose of nicorandil QD 40 mg capsule with PK sampling after administration of the drug to evaluate single-dose PK parameters of the nicorandil QD 40 mg capsule. Participants continue to receive nicorandil QD 40 mg once a day for 5 days to evaluate the steady-state PK parameters of the nicorandil QD 40 mg capsule. PK sampling is performed on day 14 to evaluate capsule-to-capsule variability and day 15 to evaluate PK parameters at steady-state. A three-day washout follows which the same cohort receives nicorandil 20 mg twice a day (Ikorel). PK sampling is performed on day 19 to evaluate single-dose PK parameters and on day 24 to evaluate steady-state PK parameters of the Nicorandil BiD 20 mg Ikorel.

PK parameters to capture may include and array of variables including but not limited to:
Plasma concentrations and time points.
Subject, period, sequence, treatment.
Intersubject, intrasubject, and/or total variability, if available.
For single-dose assessment: AUC0-t, AUC0-inf, Cmax, Tmax, λz, and t½.
For steady-state assessment: AUC0-tau, Cmaxss, Tmax, Cminss (lowest concentration in a dosing interval), Ctrough (concentration at the end of the dosing interval), Cavss (average concentration during a dosing interval), degree of fluctuation [(Cmax−Cmin)/Cavss], swing [(Cmaxss−Cminss)/Cminss]
Ctrough may be measured for several dosing intervals to assess whether steady state was achieved.

Key PK profile characteristic and comparative analysis components of the study may include but are not limited to:
Steady state pharmacokinetics for 20 mg nicorandil QD, 40 mg QD and 20 mg BiD IR nicorandil
Single dose PK profile for the 20 mg and 40 mg QD modified release nicorandil
Comparison of the singe-dose PK profile for 40 mg QD vs. two BiD doses of 20 mg BiD IR
Dose proportionality for the 20 mg and 40 mg QD single dose, especially Cₘₐₓ/D single dose, AUC_{infinity}/D and AUCₜₐᵤ/D for the single dose
Dose proportionality for the 20 mg and 40 mg QD ER steady state at multiple doses, especially Cₘₐₓ/D at steady state and AUCₜₐᵤ/D at steady state
Comparison of the steady-state exposure of 40 mg ER and 20 mg BiD IR, especially AUC₂₄ and Cₘₐₓ.

The number of subjects enrolled will be determined based on the intra-subject variability observed in an initial PK study. The study may or may not include a washout period between the administration of the 3 treatments. Each treatment period can consist of a suitable period of time (e.g., 5 days of dosing). A number of trough concentrations will be measured during each treatment period (e.g., 4 trough concentration measurements) to demonstrate attainment of steady-state conditions for each formulation. The PK profile of the major nicorandil metabolite SG-86 also may be assessed.

Steady state attainment can be evaluated by, e.g., repeated measures ANOVA for each treatment period individually using all 4 measured trough concentrations. Key PK parameters to be measured in the study can include Cmax and AUC24, e.g., both under steady state conditions. AUC24 daily exposure for BID dosing of reference formulation can be calculated as 2×AUCtau. Administration of the exemplary two-pulse controlled release nicorandil product according to the invention is anticipated to produce a similar or lower relative bioavailability than nicorandil oral tablets, defined as the upper bound of the 90% confidence interval (CI) of the geometric mean ratio (GMR; test/reference) falling at or below the upper limit of 125%.

The dose proportionality of the 20 mg two-pulse release nicorandil capsule and the 40 mg two-pulse release nicorandil capsule can be confirmed using an ANOVA statistical model for dose normalized Cmax and AUCtau at steady state with the 90% CI for GMR compared to the range of 80.00% to 125.00%, i.e., if the 2-fold increase in dose results in a similar magnitude increase in peak and overall exposure to nicorandil.

Studies also or alternatively can include a single randomized, multicenter, double-dummy, double blind, active-controlled, parallel-group, safety and efficacy (Phase 3) study of two-pulse release nicorandil once-daily capsules in patients with chronic stable angina pectoris (CSAP). Such a study can be designed to evaluate the safety and tolerability of the inventive nicorandil capsules in patients with CSAP, to demonstrate the superiority of the CR two-pulse nicorandil capsules administered QD as compared to placebo.

Study arms can be characterized by patients being randomly assigned to one of the two following study arms, with sufficient patients enrolled in each arm to demonstrate superiority of two-pulse release nicorandil QD over placebo:
Treatment A: QD Nicorandil oral capsule, 20 mg dose escalated to 40 mg QD after a defined trial period.
Treatment B: Matching QD placebo capsules equal and indistinguishable in appearance to the 20 mg and 40 mg QD nicorandil capsules in order to maintain blinding
   Patients who complete the entire study will spend a minimum of 12 weeks in the active treatment phase of the study after being enrolled, screened and undergoing washout for any anti-anginal medications they previously used for symptomatic treatment of CSAP.
a. Screening phase: This phase may include baseline ETT assessments upon enrollment, followed by a washout period to discontinue all prior antianginal medication patient may be taking. Patients may have an additional ETT-based assessment following washout to determine the baseline total exercise duration prior to randomization.
b. First treatment interval phase: Patients may spend up to 6 weeks on 20 mg modified release nicorandil QD or matching placebo.
c. Second treatment interval phase: Patient may spend an additional 6 weeks in the randomized treatment phase of the study on 40 mg modified release nicorandil QD or matching placebo.

The primary efficacy endpoint of such a study may be total exercise duration (TED) assessed during an exercise-tolerance test (ETT) using the modified Bruce protocol [Sheffield L T. Exercise stress testing. In: Braunwald E, ed. Heart Disease: A Textbook of Cardiovascular Medicine. 3rd edition. Philadelphia, PA: WB Saunders Company, 1988; 1:223-41.] approximately 6 hours post-dose. Exact timing of the assessment shall be determined following completion of a pivotal bioavailability study. ETT shall be assessed upon enrollment and—to establish TED baseline for any statistical comparison—prior to randomization and initiating of study drug (or placebo). ETT may also be conducted after adequate intervals during the study but at minimum at weeks 6 (prior to dose-escalation/up-titration), and week 12 at the end of the active treatment phase of the study. An additional ETT may be performed 48 hours after treatment conclusion in order to assess/evaluate any potential rebound effect of treatment discontinuation.

Key secondary measures of efficacy and safety (i.e., secondary endpoints) may include—but shall not be limited to—clinical variables such as time-to-onset of angina, time-to-1 mm ST-segment depression at trough, frequency of daily and/or weekly nitroglycerine (NTG) consumption, change in angina category from baseline according to the Canadian Cardiovascular Society (CCS) grading scale, frequency of weekly angina episodes, and may also include angina-related and validated patient-reported outcomes (PRO) measures such as the Seattle Angina Questionnaire (SAQ).

Further, pharmaceutical studies also or alternatively may include a single-dose, 2-sequence crossover study in healthy adult volunteers to characterize the pharmacokinetics of QD two-pulse extended-release nicorandil formulation and to provide crucial data that may be needed to inform the design of the pivotal PK study described above. A secondary goal of this exploratory PK study is to evaluate the food effect of the QD two-pulse extended-release nicorandil formulation.

The inventors conceive the studies according to these or substantially similar studies will demonstrate that two-pulse release nicorandil capsules according to the invention provide an effective QD treatment for conditions such as CSAP and exhibits pharmaceutical properties that are superior to placebo.

### Construction Principles and Description of Select Terms

This section offers guidelines for reading this disclosure.

### General Terms and Principles

The intended audience for this disclosure (“readers”) are persons having at least ordinary skill(s) in the practice of technologies discussed or used herein. Readers may also be called “skilled persons,” and such technologies and related publicly available prior knowledge are collectively referred to as “the art.” Terms such as “understood,” “known,” and “ordinary meaning” refer to the general knowledge of skilled persons.

The term “uncontradicted” means not contradicted by this disclosure, logic, or plausibility, the latter two elements being based on the knowledge of skilled persons.

Disclosed here are several different but related exemplary aspects (variations) of the invention(s) described here (also referred to as, e.g., “cases,” “facets,” “respects,” or “embodiments”). The invention encompasses all aspects as described individually and as can be arrived at by any combination of such individual aspects. Thus, any reference to “aspects” (e.g., “according to aspects” or “in an aspect”) will be understood as referring to according to any and all of the other suitable aspects described herein. In this respect, the breadth and scope of the invention should not be limited by any exemplary aspect(s)/embodiment(s). No language in this disclosure should be construed as indicating any element/step is essential to the practice of the invention unless such a requirement is explicitly stated. Uncontradicted, any aspect(s) described in any part of this disclosure can be combined with any other aspect(s) in any other part.

Uncontradicted, all technical/scientific terms used here should be read, at least in one aspect, to have the same meanings as commonly understood by skilled persons, regardless of any narrower examples or descriptions provided here (including any term introduced initially in quotations). However, readers will also recognize that some aspects can characterized by the inclusion of elements, steps, features, characteristics, etc., associated with specific descriptions provided here and that such specific disclosures represent distinct embodiments of the invention apart from the corresponding aspect that is provided by interpreting the invention using any broader commonly used terminology or concept. Uncontradicted, disclosure of any aspect using known terms, which terms are narrowed by example or otherwise, implicitly discloses one or more related aspects in which the applicable terms are alternatively interpreted using the broadest reasonable interpretation of skilled persons.

Uncontradicted, the term “or” means “and/or” here, regardless of any occasional inclusion of the actual phrase “and/or” (e.g., phrases such as “A, B, or C” and “A, B, and/or C” each simultaneously discloses aspects including (1) all of A, B, and C; (2) A and C; (3) A and B; (4) B and C; (5) only A; (6) only B; and (7) only C (and also support sub-groupings, such as “A or B,” “A or C,” etc.)).

For conciseness, symbols are used where appropriate. E.g., “&” is used for “and,” & “˜” for “about.” Symbols such as < and > are given their ordinary meaning (e.g., “≤” means “less than or equal to” & “≥” means “greater than or equal to”). A slash “/” between terms here can represent “or” (“A/B” means “A or B”) or identify synonyms of an element, depending on context. The inclusion of “(s)” after an element or a step indicates that ≥1 of such an element is present, step performed, and the like. E.g., “element(s)” refers to both 1 element and ≥2 elements, with the understanding that each thereof is an independent aspect of the invention.

Uncontradicted, the term “also” means “also or alternatively.” Uncontradicted, the terms “here” & “herein” mean “in this disclosure.” The term “i.a.” (“ia” or “ia”) means “inter alia” or “(possibly) among other things.” “Also known as” is abbreviated “aka” or “AKA.” (and can mean is otherwise referred to, even if the relationship between the terms is not well known). The term “elsewhere” means “elsewhere herein.”

Use of the abbreviation “etc.” (or “et cetera”) in association with a list of elements/steps means any or all suitable combinations of the recited elements/steps or any known equivalents of such recited elements/steps for achieving the function(s) of such elements/steps known in the art. Readers should interpret phrases like “and the like” similarly.

Uncontradicted, terms such as “and combinations,” “or combinations,” and “combinations thereof,” etc., regarding listed elements/steps, means any or all possible/suitable combinations of the associated elements/steps. Thus, e.g., uncontradicted, a phrase like “combination of any thereof” refers to any or all combinations.

Aspects may be described as suitable for use(s) disclosed herein. Uncontradicted, terms such as “suitable” or “suitability” mean acceptable, appropriate, or, in aspects practical for performing a particular function/achieving particular state(s)/outcome(s), and typically means effective, practical, and non-deleterious/harmful in the context the term is used. E.g., uncontradicted, the term “suitable” means appropriate, acceptable, or in contexts sufficient, or providing at least generally or substantially all an intended function (of the element or overall whole of the aspect), without causing or imparting significant negative/detrimental impact. Uncontradicted, each element of the invention is suitable for its intended use. In some aspects, suitability can be demonstrable through scientific studies and to a degree of significance through suitable tests/measures such as scientific tests, well-controlled and adequate studies (e.g., clinical studies), adequately powered consumer testing, and the like.

Steps, elements, devices, components, and the like also or alternatively can be described as “effective.” Uncontradicted, any disclosed element is to be construed as being effective for its intended purpose and present in an effective amount, and any step performed is to be understood as being performed/applied effectively, such as in an effective amount or an effective number of times, etc. Uncontradicted efficacy can be judged by evaluating the element(s) ability to perform or contribute to the described function(s) or characteristic(s) associated with the component, device, step, etc., the overall aspect, or both. Efficacy in this respect when applied to effects in organisms, such as people, can mean, and, uncontradicted, should be interpreted to implicitly disclose efficacy that can be measured (1) in a treated subject, (2) in a majority of subjects in a population, (3) in a statistically significant number of subjects in a population, (4) generally all subjects in a population, (5) substantially all subjects in a population, or (6) in a statistically significant number of or more of a typical or average subject of the class of subjects treated. Object elements are, uncontradicted, present in “effective amount,” and, uncontradicted, any described class of excipient or specific excipient is understood to be present in the associated composition/formulation in an effective amount, which generally means, an amount that is effective for the described function(s) associated with the element.

Uncontradicted, heading(s) (e.g., “Construction and Terms”) and subheadings used here are included for convenience and do not limit the scope of any aspect(s). Uncontradicted, aspect(s), step(s), or element(s) described under one heading can apply to other aspect(s) or step(s)/element(s) here.

Ranges of values here represent each value falling within a range within an order of magnitude of the smallest endpoint of the range without having to write each value of the range explicitly. E.g., a recited range of 1-2 implicitly discloses each of 1.0, 1.1, 1.2, . . . 1.9, and 2.0, and 10-100 implicitly discloses each of 10, 11, 12, . . . 98, 99, and 100). Uncontradicted, all ranges include the range's endpoints, regardless of how a range is described. E.g., “between 1-5” includes 1 and 5 in addition to 2, 3, and 4 (and all numbers between such numbers within an order of magnitude of such endpoints, e.g., 1.0, 1.1, . . . 4.9, and 5.0). For the avoidance of doubt, any number within a range, regardless of the order of magnitude of the number, is covered by the range (e.g., a range of 2-20 covers 18.593). Uncontradicted, readers will understand that any two values in a range provided as a list herein can be combined as endpoints to form a range defining a more particular aspect of the invention (e.g., if a list of values 1, 2, 3, 4, and 5 of element X is provided, readers will understand that the disclosure implicitly discloses an aspect comprising 2-4×, 3-5×, and 1-3×, etc.

Terms of approximation (e.g., “about,” “˜,” or “approximately”) can be used here (1) to refer to a set of related values or (2) where a precise value is difficult to define (e.g., due to limits of measurement). Uncontradicted, all exact values provided here simultaneously/implicitly disclose corresponding approximate values and vice versa (e.g., disclosure of “about 10” provides explicit support for the use of 10 exactly in such aspect/description). Ranges described with approximate value(s) include all values encompassed by each approximate endpoint, regardless of presentation (e.g., “about 10-20,” (or, e.g., “˜10-20”) has the same meaning as “about 10-about 20” (or, e.g., “˜10-˜20”). The scope of value(s) encompassed by an approximate term typically depends on the context of the disclosure, criticality or operability, statistical significance, understanding of the art, etc. In the absence of guidance here or in the art for an element, terms such as “about” when used in connection with an element should be interpreted as ±10% of the indicated value(s) and implicitly disclosing ±5%, ±2%, ±1%, and ±0.5%.

This disclosure includes aspects associated with particular characteristics, such as amounts of components (or ranges thereof), In cases, several such characteristics of varying scope may be provided. Readers will understand that each such characteristic can be associated with particular properties that distinguish such aspects from other aspects, and, accordingly, each such range can be viewed as critical to a particular aspect of the invention, even if the associated results, properties, functions, etc., associated with such aspects are not directly communicated in association with such characteristics.

Lists of aspects, elements, steps, and features are sometimes employed for conciseness. Unless indicated, each member of each list should be viewed as an independent aspect. Each aspect defined by any individual member of a list can have and often will have, nonobvious properties vis-a-vis aspects characterized by other members of the list.

Uncontradicted, the terms “a” and “an” and “the” and similar referents encompass both the singular and the plural form of the referenced element, step, or aspect. Uncontradicted, terms in the singular implicitly convey the plural and vice versa herein (in other words, disclosure of an element/step implicitly discloses the corresponding use of such/similar elements/steps and vice versa). Hence, e.g., a passage regarding an aspect including X step supports a corresponding aspect including several X steps. Uncontradicted, any mixed use of a referent such as “a” in respect of one element/step or characteristic and “one or more of” with respect to another element/step or characteristic in a paragraph, sentence, aspect, or claim, does not change the meaning of such referents. Thus, for example, if a paragraph describes a composition comprising “an X” and “one or more Ys,” the paragraph should be understood as providing disclosure of “one or more Xs” and “one or more Ys.”

“Significant” and “significantly” mean results/characteristics that are statistically significant using ≥1 appropriate test(s)/trial(s) in the given context (e.g., p≤0.05/0.01). “Detectable” means measurably present/different using known detection tools/techniques. The acronym “DOS” (or “DoS”) means “detectable(ly) or significant(ly).” The term “measurably” means at a measurable level. The term detectable provides implicit disclosure for aspects that are “measurable,” and the term “measurable” implicitly supports aspects where the measured or measurable element is “detectable.”

Uncontradicted, any value provided here that is not accompanied by a unit of measurement (e.g., a weight of 50 or a length of 20), either any previously provided unit for the same element/step or the same type of element/step will apply, or, in cases where no such disclosure exists, the unit most used in association with such an element/step in the art applies.

Uncontradicted, the terms “including,” “containing,” “comprising,” and “having” mean “including, but not limited to,” or “including, without limitation.” Uncontradicted, use of terms such as comprising and including regarding elements/steps means including any detectable number or amount of an element or including any detectable performance of a step/number of steps (with or without other elements/steps). Uncontradicted, “a” means one or more, even when terms such as “one or more” or “at least one” are used in association with the referent “a.”

For conciseness, description of an aspect “comprising” or “including” an element, with respect to a collection/whole (e.g., a system, device, or composition), implicitly provides support for any detectable amount/number or ≥˜1%, ≥˜5%, ≥˜10%, ≥˜20%, ≥˜25%, ≥˜33%, ≥˜50%, ≥˜51%, ≥˜66%, ≥˜75%, ≥˜90%, ≥˜95%, ≥˜99%, or ˜100% of the whole/collection being made up of the element, or essentially all of the whole/collection being made up of the element (i.e., that the collection consists essentially of the referenced element). Similarly, a method described as including a step with respect to an effect/outcome implicitly provides support for the referenced step, providing ≥˜1%, ≥˜5%, ≥˜10%, ≥˜20%, ≥˜25%, ≥˜33%, ≥˜50%, ≥˜51%, ≥˜66%, ≥˜75%, ≥˜90%, ≥˜95%, ≥˜99%, or ˜100% of the effect/outcome, representing ≥˜1%, ≥˜5%, ≥˜10%, ≥˜20%, ≥˜25%, ≥˜33%, ≥˜50%, ≥˜51%, ≥˜66%, ≥˜75%, ≥˜90%, ≥˜95%, ≥˜99%, or ˜100% of the steps/effort performed, or both. Explicit listing of percentages of elements in connection with particular aspects does not limit or contradict such implicit disclosure. Uncontradicted, readers should interpret terms such as “essentially all” or “essentially” consistent with the concept of “consisting essentially of.”

Uncontradicted, terms such as “comprising” when used in connection with a step of a method provide implicit support for performing the step once, ≥2 times, or until an associated function/effect is achieved.

Uncontradicted, any disclosure of an object or method (e.g., composition, device, or system) “comprising” or “including” element(s) provides implicit support for an alternative corresponding aspect that is characterized by the object consisting of that element or “consisting essentially of” that element (excluding anything that would “materially affect” the “basic and novel characteristic(s)” of the invention). Uncontradicted, any specific use of phrases such as “consists of” and “consists essentially of” herein does not modify this construction principle.

Readers will understand the “basic and novel characteristic(s)” of the invention and the scope of what constitutes a “material effect” (or “material effect”) of such “basic and novel characteristics” will vary with the specific applicable aspect of the invention at issue. Uncontradicted, the basic and novel characteristics of any aspect of the invention include the specific recited and associated elements of an aspect and exclude any other element that significantly detracts from the intended function(s) of the recited elements, that introduce significant new functions that are unrelated to the intended function(s), that significantly reduce the performance of the function(s), or that significantly negatively change other characteristics of performing such function(s) (e.g., by increasing the cost of performing the functions in energy, money, or both). Uncontradicted, the basic and novel characteristics also include at least significantly retaining the suitability, effectiveness, or both, of recited elements or the overall aspect. Accordingly, a material effect can be an effect that reduces, diminishes, eliminates, counteracts, cancels, or prevents one or more of such functions in one or more respects (e.g., delaying onset, reducing scope, reducing duration, reducing output, reducing the level of applicability, reducing effect, or combinations thereof). In an aspect, a material effect is one that changes such functions by making such functions impractical, difficult to obtain, or materially more expensive or otherwise costly in terms of inputs. From this and the other guidance provided herein, readers can understand the scope of an aspect that is defined by consisting essentially of a collection of elements.

Uncontradicted, the term “one” means a single type, single iteration/copy/thing, of a recited element or step, or both, which will be clear from the context of the relevant disclosure. For example, the referent “one” used with respect to a component of a composition/article or system can refer to one type of element (which may be present in numerous copies, as in the case of an ingredient in a composition) one unit of the element, or both. Similarly, “one” component, a “single” component, or the “only component” of a system typically means 1 type of element (which may be present in numerous copies), 1 instance/unit of the element, or both. Further, “one” step of a method typically means performing one type of action (step), one iteration of a step, or both. Uncontradicted, a disclosure of “one” element provides support for both, but uncontradicted, any claim to any “one” element means one type of such an element (e.g., a type of component of a composition/system/article).

Uncontradicted, the term “some” means ≥2 copies/instances or ≥5% (e.g., ≥7.5%, ≥12.5%, ≥17.5%, ≥27.5%, or ≥37.5%) of a listed collection/whole is or is made up of an element. Regarding methods, some means ≥5% of an effect, effort, or both is made up of or is attributable to a step (e.g., as in “some of the method is performed by step Y”) or indicates a step is performed ≥2 times (e.g., as in “step X is repeated some number of times”). Terms such as “predominately,” “most,” or “mostly” (and “primarily” when not used to refer to an order of events or “mainly”) means detectably >50% (e.g., mostly comprises, predominately includes, etc., mean >50%) (e.g., a system that mostly includes element X is composed of >50% of element X). The term “generally” means ≥75% (e.g., generally consists of, generally associated with, generally comprises, etc., means ≥75%) (e.g., a method that generally consists of step X means that 75% of the effort or effect of the method is attributable to step X). “Substantially” or “nearly” means ≥95% (e.g., nearly all, substantially consists of, etc., mean ≥95%) (e.g., a collection that nearly entirely is made up of element X means that at least 95% of the elements in the collection are element X). Terms such as “generally free” of an element or “generally lacking” an element mean comprising ≤25-% of an element, and terms such as “substantially free” of an element mean comprising ≤˜5% of an element. Uncontradicted, any aspect described as “generally comprising” or “generally consisting” of an element implicitly discloses an element that “substantially comprises” the element. The same principle applies to any disclosure where an aspect is described as being “generally free” of an element.

Uncontradicted, phrases such as “substantially identical” or “substantially similar” may be used to refer to element(s)/component(s)/ingredient(s)/thing(s) (e.g., composition, system, device, etc.) or step(s)/method(s) that have the same or about the same characteristic(s) or achieve the same or about the same result(s), typically in a similar way, as a referenced element/thing or step/method or otherwise do not meaningfully differ in intended result and manner of achieving such a result or are otherwise recognized in the art as not differing or not differing substantially in the relevant context (e.g., by being considered equivalents). Uncontradicted, readers will understand that a “substantially identical” or “substantially similar” element/thing or step/method when compared to a comparator thing/element or method/step means that the referenced element/thing or step/method exhibits such a similar function as a comparator at identical, approximately identical, or statistically similar amounts as the comparator thing or method when applied under similar conditions of use. Again, where statistical, approximate, or other measured comparisons are not possible, readers will understand the phrase as encompassing those things known as being identical or substantially identical to the referenced element/step or are described as such herein.

Uncontradicted, any aspect described with respect to an optionally present element(s)/step(s) also provides implicit support for corresponding aspect(s) in which one, some, most, generally all, nearly all, essentially all, or all such element(s) are lacking/step(s) not performed, in respect of the relevant aspect. E.g., disclosure of a system comprising element X implicitly also supports a system lacking element X. That is, readers will understand that any element, feature, step, or characteristic of any aspect of the invention recited herein as being present in an aspect also implicitly provides support for the element, feature, step, or characteristic as being excluded from a corresponding/similar aspect of the invention implicitly disclosed by the explicit positive disclosure. Uncontradicted, changes to tense or presentation of terms (e.g., using “comprises predominately” in place of “predominately comprises”) do not change the meaning of the corresponding term/phrase.

Uncontradicted, all methods provided here can be performed in any suitable order regardless of presentation (e.g., a method comprising steps A, B, and C can be performed in the order C, B, and A; B and A and C simultaneously, etc.). Uncontradicted, elements of a composition can be assembled in any suitable manner by any suitable method. In general, any methods and materials similar or equivalent to those described here can be used in the practice of embodiments in at least the broadest version of the relevant aspect. Uncontradicted, the use of ordinal numbers such as “first,” “second,” “third,” and so on is primarily, though not exclusively, intended to distinguish respective elements rather than to limit the disclosure to a particular order of those elements, importance, or configuration.

Any elements associated with a function can be alternatively described as “means for” performing a function in a composition/device/system or a “step for” performing a part of a method, and parts of this disclosure refer to “equivalents,” which means known equivalents known in the art for achieving a referenced function associated with disclosed mean(s)/step(s). However, no element of this disclosure or claim should be interpreted as limited to a “means-plus-function” or “step-plus-function” construction unless such intent is clearly indicated by the use of the terms “means for” or “step for.” Terms such as “configured to” or “adapted to” do not indicate “means-plus-function” interpretation but, rather, describe element(s)/step(s) configured to, designed to, selected to, or adapted to achieve a certain performance, characteristic, property, or the like using teachings provided here or in the art.

As used herein, the word “exemplary” means “serving as an example, instance, or illustration.” Any embodiment described herein as “exemplary,” “representative,” or “illustrative,” etc., should not necessarily be construed as preferred or advantageous over other embodiments. Any embodiment described herein as “exemplary” is not necessarily to be construed as preferred or advantageous over other embodiments.

In general, except where explicitly indicated or clearly indicated by context, terms such as “improved” or “better” mean DOS “increased.” In some respects, terms such as “improved” or “better” mean DOS “reduced,” such as with respect to reducing negative elements of a method or composition. Uncontradicted, terms such as “enhanced,” “improved,” “better,” and the like are used synonymously. Additional description of such terms is provided elsewhere herein.

All references (e.g., publications, patent applications, and patents) cited herein are hereby incorporated by reference as if each reference were individually and specifically indicated to be incorporated by reference and set forth in its entirety herein. Uncontradicted, any suitable principles, methods, or elements of such references (collectively “teachings”) can be combined with or adapted to aspects. However, citation/incorporation of patent documents is limited to the technical disclosure thereof and does not reflect any view regarding the validity, patentability, etc., thereof. Uncontradicted, in the event of any conflict between this disclosure and the teachings of such documents, the content of this disclosure takes precedence regarding interpreting aspects of the invention. Numerous references are cited here to concisely incorporate known information and aid skilled persons in putting aspects into practice. While efforts have been made to include the most relevant references for such purposes, readers will understand that not every aspect of every cited reference will apply to every aspect of the invention.

While elements disclosed in such incorporated references can be combined with aspects of the invention disclosed herein, readers will understand that the invention is intended to stand apart from such disclosures and, accordingly, uncontradicted, in aspects, any element(s) of the objects or methods of any such references are excluded from the scope of the invention (e.g., if reference A discloses object or element B, any aspect that is not directed to object or element B can be characterized by, as one aspect, the lack of object or element B).

All original claims contained in this disclosure, when filed, are incorporated into this specification as if they were a part of the description.

### Terms and Principles Specific to this Disclosure

The following description of certain terms and acronyms is provided to assist readers in understanding the invention. Additional acronyms may be only provided in other parts of this disclosure and acronyms that are well known in the art may not be provided here.

Uncontradicted, any description of weight of an element or component (e.g., of a composition or of an ingredient) is in reference to weight percent (sometimes alternatively presented as “wt. %” or “wt. %”).

The term “NCD” is sometimes used herein to refer to “nicorandil.” The term “NCDC” is sometimes used herein to refer to a nicorandil compound (described elsewhere).

The term “CSAP” is sometimes used herein to refer to “chronic stable angina pectoris.”

Except where explicitly indicated or clearly indicated by context, “improved” herein means “increased” in terms of favorable characteristics (e.g., amount of nicorandil present at 12, 14, 16, or 18 hours post administration, the duration of significant increases in cardiovascular blood vessel dilation/enlargement following administration, etc.) and “reduced” in terms of negative characteristics (e.g., the amount of nicorandil released within 1, 2, or 3 hours post administration, the frequency/severity of adverse events, toxicity, etc.). Uncontradicted, terms such as “enhanced,” “improved,” and the like are used synonymously.

“Pharmaceutical suitability”, “pharmaceutically suitable,” “pharmaceutically acceptable,” and similar phrases, typically used to refer to compositions that are safe and effective for pharmaceutical administration and application, having sufficient potency, purity, strength, quality, and safety for pharmaceutical application, in cases specifically to the eye, as may be judged by regulatory authority review, and as established by, e.g., one or more well controlled and adequate clinical studies performed in compliance with generally prevailing regulatory authority standards. Uncontradicted, a description of “suitability” implicitly means that the referenced element, step, etc., is pharmaceutically suitable or otherwise medically suitable (e.g., safe and effective as determined by proper nonclinical/clinical testing).

Excipients and active ingredients described herein will be understood to typically be present in “effective amounts,” and, uncontradicted, any described class of active ingredient, excipient class, collection of excipients, or specific excipient is understood to be present in the associated composition/formulation in effective amount(s), which generally means, in this context, an amount that is effective for the described function(s) associated with the referenced active ingredient or excipient (it being understood that some excipient compound(s)/ingredient(s) exhibit more than one effect). E.g., a tonicity agent will be understood to be present in a composition/formulation in an amount that is effective to impart an indicated tonicity effect, a tonicity effect that is required for suitability of the composition, or an effect that imparts a significant tonicity effect on a composition, or, e.g., herein a binding agent or a controlled-release agent being present in a composition/formulation in an amount that is effective to impart a binding or release-control effect, respectively, (with respect to comparator composition(s) lacking the compound(s)/ingredient(s)).

A portion of a composition, such as a portion mostly or generally comprising or consisting of a class/type of excipient or a can be referred to “component” herein—e.g., a “disintegrant/binder component” may include or more types of binders, disintegrants, or both). Excipients also can be described in terms of ingredients, which can comprise a combination of two or more types of compounds (e.g., an SMCC, such as a PROSOLV excipient, comprises both microcrystalline cellulose and colloidal silicone). Readers will understand that some excipient compound(s)/ingredient(s) can exhibit more than one effect). E.g., a binder may act as a disintegrant and vice versa.

Pharmacokinetic terms used in this disclosure are generally understood in the art and any descriptions of such terms provided herein are either to illustrate such terms or describe specific exemplary understandings of such terms without limiting the scope thereof. The term “Cₘₐₓ” (sometimes presented as Cmax) is generally understood as maximum observed blood plasma concentration or the maximum blood plasma concentration calculated or estimated from a concentration to time curve, and is typically expressed in units of ng/ml. The term “Tₘₐₓ” is understood to mean the time after administration at which Cₘₐₓ occurs and is usually expressed in units of hours (h). The term “steady state” is understood to refer to a state where the blood plasma concentration curve for a given drug (here NCDC(s)) does not substantially fluctuate after repeated doses to dose of the formulation. Relative bioavailability typically is measured as the concentration in the blood (serum or plasma) versus time area under the curve (AUC) determined for the test composition divided by the AUC in the blood provided by the reference composition. Bioequivalence is understood to typically means that in respect of two treatments the 90% confidence interval of the ratio of a log-transformed exposure measure (AUC and/or Cₘₐₓ) falls completely within the range 80-125%.

Uncontradicted, terms such as “carrier” and “excipients” mean any physiologically inert, pharmacologically inactive material known to one skilled in the art, which is compatible with the physical and chemical characteristics of NCDC(s), generally, or the particular NDCD(s) described in connection therewith as an active ingredient. Pharmaceutically acceptable excipients include polymers, resins, plasticizers, fillers, binders, lubricants, glidants, disintegrants, suspending agents, deflocculating agents, solvents, co-solvents, buffer systems, surfactants, preservatives, sweetening agents, flavoring agents, pharmaceutical grade dyes or pigments, and viscosity agents, and others exemplified/described herein, any or all of which can be present, in suitable, in compositions of the invention, in effective amounts.

In aspects the term “carrier” or “vehicle” is alternatively used to describe a component of a composition that is directed primarily or entirely to the delivery of other components (e.g., dosage forms). E.g., a carrier might be a capsule that carries one, two, three, four, or more tablet dosage forms comprising 1, 2, or more formulations. The term “delivery facilitating component” is sometimes used to avoid confusion with such terms. Such a component is also frequently exemplified herein as a capsule, though other alternative forms for carrying NCDC-containing material alternatively can be used where suitable.

Exemplary components suitable for pharmaceutical compositions described herein may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N. Y., 1980; and Pharmaceutical Dosage Forms and Drug Delivery Systems, Seventh Ed. (Lippincott Williams & Wilkins 1999), herein incorporated by reference in their entirety.

The term “composition” unless contradicted means a composition of the invention, comprising one or more NCDCs and a controlled/modified release formulation that exhibits one or more of the various physiochemical properties, physiological/therapeutic effects, compositional characteristics, and the like described throughout this disclosure.

Uncontradicted, the term “standard” as used herein with respect to techniques or components/elements not further described or exemplified herein (e.g., standard stability testing conditions) means either the standard applied by leading regulatory authorities (e.g., US FDA, the EU EMA, the Chinese NMPA, Japan's PMDA, Health Canada, India's CDSCO, and the like) or that is most commonly used in the art.

Uncontradicted, a “therapeutically effective amount” of an active pharmaceutical ingredient (API), here typically NCDC(s), or composition, typically means an amount of a compound or pharmaceutical composition that will elicit an intended (typically significant) biological or medical response of a tissue, system, animal, or human that is being sought by the researcher, medical doctor, or other clinician. In aspects, a therapeutically effective amount is demonstrated by at least one or at least two well controlled and adequate clinical studies in human subjects/patients (e.g., as would be considered sufficient for pharmaceutical approval). Herein, “clinically relevant efficacy” typically means demonstrating a detectable or significant intended effect or biomedical or medical response (typically significant) in or on a targeted tissue, system, animal, or human sought by a researcher, medical doctor, or other clinician. Herein, a “clinically relevant level” of a drug is a level sufficient to yield a detectable or significant intended effect or biomedical or medical response (typically significant) in or on a targeted tissue, system, animal, or human. Any composition described herein in such terms will be understood as disclosing aspects having the features provided in this paragraph.

Terms such as “pharmaceutical composition,” “formulation,” and the like, or components thereof (e.g., sub-composition dosage forms, components, or portions), when used to describe compositions generally described or referenced herein, includes any suitable dosage form, typically solid dosage forms such as granules, multiunit particulate systems (MUPS), pellets, spheres, tablets, dispersible tablets, soft capsules, hard capsules, mini-tablets, beads, particles and the like; and liquid dosage forms such as solutions, suspensions, emulsions, colloids and the like, which can, in aspects, be adapted for oral administration.

Terms such as “pH responsive” and “pH sensitive” herein when used to refer to a component, excipient/ingredient, or composition mean that the referenced element exhibits a detectable or significant change in composition, function, characteristics, etc., based on different pH conditions (e.g., the presence of highly acidic conditions (at a pH of about 0.5-3, such as 0.75-2.5, or 0.5-2, 0.75-2.25, 0.5-1.5, 0.75-1.5, 0.8-1.4, or 1-1.5, e.g., around 1.2 or 1.2), neutral/near neutral conditions (e.g., a pH of about 6.5-7.5 such as about 7), or alkaline conditions (e.g., a pH of about 8, 8.5, 9, or greater), or the presence of conditions associated with one or more parts of the intestine (e.g., as illustrated in FIG. 26). Elements that are not pH responsive can be described as pH insensitive, general, or pH independent, etc.

The terms “Ikorel” and “Nikoran” and various forms thereof (sometimes capitalized, sometimes not), refer to either the products as commercially sold by their respective producers (Sanofi or Torrent Pharmaceuticals, respectively) under these brand names as the filing date of this application (and where applicable as described in associated regulatory filings/market authorization documents). Readers will understand that each such term can be substituted with the corresponding formulation, where comparison is made to the performance of such formulations in respect of NCDC(s) that may or may not be limited to nicorandil.

Compositions provided herein can, in aspects, be characterized as “modified” release compositions. The term “modified” in this context refers to the different characteristics of such compositions as compared to leading nicorandil formulations (e.g., Ikorel and Nikoran).

Compositions herein can comprise components that can be described as “controlled” release components/formulations or “sustained release” component/formulations. These terms are likewise used to describe the relatively longer release of NCDC(s) from such formulations as compared to current leading on-market products, as exemplified in the Examples provided below. Uncontradicted, the term “controlled release” means the time course of NCDC appearance/release in medium surrounding the composition containing the NCDC is DOS modified as compared to another composition, typically an immediate release composition (e.g., an Ikoreal formulation composition). Readers will understand that terms such as “controlled release” encompass or at least provide implicit support for “delayed release” and “extended release” formulations/elements. Uncontradicted, the term “extended release” refers to compositions which are characterized by having a detectable or significant release over a period of at least ˜12, ˜14, or ˜18 hours after administration to a subject, such as a human patient. Compositions of the invention, as exemplified herein, can be characterized by such properties. A controlled release typically means a release that is predictable for at least a significant period of time, such as about 20-80%, 25-75%, 20-60%, 25-50%, 15-60%, 15-75%, 20-33%, 15-33%, or 12.5%-50% of the time that the NCDC(s) are detectably or significantly (DOS) released from the relevant material (component, formulation, matrix, etc.). In aspects, a controlled release formulation can be characterized as an extended/sustained release component (e.g., having a release profile reflecting a significantly longer release than an immediate release, a faster release component, or both) a delayed release (e.g., a release profile reflecting a significant delay in the amount of release, relate of release, etc., as compared to an immediate release form, faster release form, or both), or both, or can comprise a component describable as such. In aspects, controlled release compositions/formulations can comprise a component that can be characterized as an immediate release, early release, or rapid release component. In any case, such a controlled release can be provided/obtained through, inter alia, the use of controlled release formulations as described herein and using equivalent means known in the art or ingredients having the same functional properties as those described in the various embodiments and aspects provided here.

In aspects, “controlled release” formulations/components exhibit, i.a., a release rate that is predetermined in order to maintain an approximate/significantly constant drug concentration for a pre-determined period of time, while minimizing side effects (e.g., meeting a target profile/release, as exemplified in the Examples herein).

In aspects, a modified (e.g., sustained release, delay release, or delayed and sustained release) component/composition is provided by a composition or used in a composition of the invention, which, in either case, can be distinguished from, e.g., an immediate release profile, which typically demonstrates dissolution without significant delay or pause, and wherein the drug is effectively available immediately upon administration for absorption and pharmacologic action.

Uncontradicted, an “immediate release composition” herein is a composition that exhibit significant release within minutes of administration and typically achieve ≥85% of release within ˜30 minutes of administration.

Still alternatively formulations may be described as “intestinal release targeting” or “stomach release targeting” (“upper gastrointestinal (GI) tract release targeting”). Such terms are not necessarily intended to refer to any particular specific affinity elements/components, but, rather, that given dissolution characteristics of such compositions most, generally all, or substantially all of the NCDCs in such formulations/components will be released in such parts of the GI tract of humans.

In aspects, a controlled release formulation also or alternatively can be characterized on the basis of mostly, generally only, or essentially only releasing API(s) upon certain conditions, such as under certain pH conditions for a period of time.

In this respect, elements/formulations can be described as “gastric acid dissolution susceptible” (GADS) or “gastric acid dissolution resistant” (GADR). E.g., compositions can comprise a GADS component (a GADSC) and a GADR component (GADRC). The GADS designation is used to refer to formulations/elements that are susceptible to dissolution in gastric acid conditions (e.g., at a pH of about 1.2), by, e.g., most, generally all, or substantially all of the NCDC(s) in the GADS element/component being released under such conditions in about 2-7 hours of contact with such a dissolution medium (whether in vitro or in vivo, the latter reflecting normal acidic conditions of the stomach). In contrast, a GADR element/component will exhibit significantly less dissolution in gastric acid conditions, such as by most, generally all, or substantially all/essentially all or all of the NCDC in the GADR element/component not being subject to dissolution when the GADR element/component is in contact with such a dissolution media. Each or both of the GADRC and GADC of a composition can comprise one or more, in aspects a plurality, of dosage forms, e.g., tablets.

Solid dosage form formulations can, in aspects, be characterized as “matrix” formulations or as comprising a matrix formulation. The term “matrix” as applied to a formulation, component, or composition of the invention is understood as referring to a drug delivery system in which drugs (here NCDC(s)) is/are dispersed, either molecularly or as solid drug particles, within a polymer network. Such a network can be formed by the combination of multiple different types of polymers (e.g., starch binders and cellulosic polymer-controlled release polymers) and can be present in any suitable form. The term “network” and “matrix” are, accordingly, understood by readers as not limiting such formulations to compositions with any particular types of structural characteristics other than those provided in this disclosure or inherently present in the disclosed formulations and recognizable by those of skill in the art.

Any suitable solid formulation or dosage form disclosed herein, in aspects, can be characterized as comprising such a matrix formulation (which can be referred to as, e.g., a controlled release matrix formulation).

In cases, elements/components are described in references to “pulses” of release. E.g., a composition of the invention can exhibit a first release/pulse under acidic conditions, such as in gastric acid dissolution conditions, and a second pulse/release, such as under intestinal like conditions (at higher pHs, e.g., of at least about 5.5 or higher). Accordingly, compositions/formulations described herein, in aspects, are referred to as “two pulse” or “two pulse release” compositions/formulations.

In view of the different but often overlapping characteristics of elements/components of compositions of the invention, in cases this description refers to components, elements, dosage forms, etc., that have different formulations (e.g., first formulation and second formulation) or that have different functional properties (a first controlled release formulation and a second controlled release formulation). Readers will understand that such terms are used to describe formulations with different, often substantially different, properties and performance characteristics, which can include some or all of the other characteristics described in this section.

We claim:

1. A pharmaceutically acceptable composition comprising a therapeutically effective amount of nicorandil contained in a controlled release formulation comprising a controlled release gastric acid dissolution susceptible component comprising a release retardant polymer component and a controlled release gastric acid dissolution resistant component comprising both a release retardant polymer component and a pH responsive release resistant polymer component, wherein (1) upon maintaining the composition in contact with a pH 1.2 dissolution media for a period of 4 hours the composition releases no more than 50% of the nicorandil contained in the composition, (2) the gastric acid dissolution susceptible controlled release component (a) comprises a first part of the therapeutically effective amount of the nicorandil, (b) releases (i) between 11% to 28% of the nicorandil initially present in the gastric acid dissolution susceptible component after 0.25 hours in the dissolution media of pH 1.2; (ii) 35% to 50% of the nicorandil initially present in the gastric acid dissolution susceptible component after 1 hour in the dissolution media of pH 1.2; (iii) 55% to 70% of the nicorandil initially present in the gastric acid dissolution susceptible component after 2 hours in the dissolution media of pH 1.2; and (iiii) 80-100% of the nicorandil initially present in gastric acid dissolution susceptible component 6 hours after contact with the dissolution media having a pH of about 1.2, (3) the gastric acid dissolution resistant component (a) comprises a second part of the therapeutically effective amount of the nicorandil and (b) releases no more than 20% of the nicorandil contained in the gastric acid dissolution resistant component after 6 hours of contact with a dissolution media having a pH of 1.2, and (4) the amount of the nicorandil in the first part of the therapeutically effective amount and the amount of the nicorandil in the second part of the therapeutically effective amount differ by less than about 10%, and wherein the first part comprises 30-70% of the nicorandil in the composition.

2. The composition of claim 1, wherein (1) the gastric acid dissolution susceptible component comprises a matrix formulation comprising 25-45% of a release retardant polymer component and 40-70% of a binder component, (2) the gastric acid dissolution resistant component comprises a matrix formulation comprising 25-45% of a release retardant polymer component and 40-70% of a binder component, or (3) the gastric acid dissolution susceptible component comprises a matrix formulation comprising 25-45% of a release retardant polymer component and 40-70% of a binder component, and the gastric acid dissolution resistant component comprises a matrix formulation comprising 25-45% of a release retardant polymer component and 40-70% of a binder component.

3. The composition of claim 2, wherein (1) the release retardant polymer component and the nicorandil in the gastric acid dissolution susceptible component are present in a ratio of 2:1 to 5:1, (2) the release retardant polymer component and the nicorandil in the gastric acid dissolution resistant component are present in a ratio of 2:1 to 5:1, or (3) the release retardant polymer component and the nicorandil in the gastric acid dissolution susceptible component are present in a ratio of 2:1 to 5:1 and the release retardant polymer component and the nicorandil in the gastric acid dissolution resistant component are present in a ratio of 2:1 to 5:1

4. The composition of claim 2, wherein (1) 40-65% of the gastric acid dissolution susceptible component is composed of a binder component, (2) 40-65% of the gastric acid dissolution resistant component is composed of a binder component, or (3) 40-65% of the gastric acid dissolution susceptible component is composed of a binder component and 40-65% of the gastric acid dissolution resistant component is composed of a binder component.

5. The composition of claim 4, wherein (1) the binder component and the nicorandil are present in a ratio of about 4:1 to about 7:1 in the gastric acid dissolution susceptible component, (2) the binder component and the nicorandil are present in a ratio of about 4:1 to about 7:1 in the gastric acid dissolution resistant component, or (3) the binder component and the nicorandil are present in a ratio of about 4:1 to about 7:1 in the gastric acid dissolution susceptible component and the binder component and the nicorandil are present in a ratio of about 4:1 to about 7:1 in the gastric acid dissolution resistant component.

6. The composition of claim 1, wherein most of the excipients in the composition have a moisture content of less than 6%.

7. The composition of claim 1, wherein the gastric acid dissolution susceptible component comprises 2-5 gastric acid dissolution susceptible component dosage forms and the gastric acid dissolution resistant component comprises 2-5 gastric acid dissolution resistant component dosage forms, wherein gastric acid dissolution susceptible dosage forms and the gastric acid dissolution resistant dosage forms are contained in a pharmaceutically acceptable capsule.

8. The composition of claim 1, wherein the therapeutically effective amount is 15-75 mg of nicorandil.

9. The composition of claim 8, wherein the nicorandil is the only active pharmaceutical ingredient of the composition.

10. The composition of claim 1, wherein the nicorandil is the only active pharmaceutical ingredient of the composition.

11. The composition of claim 10, wherein the gastric acid dissolution susceptible component comprises 2-5 gastric acid dissolution susceptible component dosage forms and the gastric acid dissolution resistant component comprises 2-5 gastric acid dissolution resistant component dosage forms, wherein gastric acid dissolution susceptible dosage forms and the gastric acid dissolution resistant dosage forms are contained in a pharmaceutically acceptable capsule.

12. A pharmaceutical product comprising the pharmaceutical composition of claim 1 packaged in an aluminum blister pack, the aluminum blister pack further containing an effective amount of a pharmaceutically acceptable desiccant.

13. A pharmaceutical product comprising the pharmaceutical composition of claim 7 packaged in an aluminum blister pack, the aluminum blister pack further containing an effective amount of a pharmaceutically acceptable desiccant.
